(19) United States
(12) Patent Application Publication
(10) Pub. No.: US 2025/0327755 A1
(43) Pub. Date: Oct. 23, 2025

(54) PRODUCTION SYSTEM FOR THE MANUFACTURE OF A PRODUCT

(71) Applicant: KyooBe Tech GmbH, Leinfelden-Echterdingen (DE)

(72) Inventors: Lena SCHOBER, Grossbottwar (DE); Mario BOTT, Dettingen unter Teck (DE)

(21) Appl. No.: 19/228,989

(22) Filed: Jun. 5, 2025

Related U.S. Application Data
(63) Continuation of application No. PCT/EP2023/084583, filed on Dec. 6, 2023.

(30) Foreign Application Priority Data
Dec. 12, 2022 (DE) 10 2022 132 935.6

(51) Int. Cl. G01N 21/88; G01N 21/89; G01N 29/44

(52) U.S. Cl. CPC G01N 21/8803; G01N 21/89; G01N 29/44

(57) ABSTRACT

A production system for manufacturing a product, in particular a biological-pharmaceutical product, which comprises multiple processing units for accommodating and/or treating an educt, and/or to a method for this purpose.

### RELATED APPLICATION

This application is a continuation of international application No. PCT/EP2023/084583 filed on Dec. 6, 2023, and claims the benefit of German application No. 10 2022 132 935.6 filed on Dec. 12, 2022, which are incorporated herein by reference in their entirety and for all purposes.

### FIELD OF DISCLOSURE

The present invention relates to a production system for manufacturing a product. In particular, the present invention relates to a production system for manufacturing a biological-pharmaceutical product.

### BACKGROUND

Conventional production systems, in particular in the pharmaceutical sector for personalized therapeutics, have a high proportion of manual interventions. These interventions do enable these therapeutics to be manufactured on a highly personalized level or in a highly personalized manner, however, high costs are linked to such individual and/or ultrasmall productions and high variations of the quality as well as, accompanying this, high levels of production rejects and/or bad parts are registered. Furthermore, there is no possibility for a large-scale application and/or an industrial production of individual and/or ultrasmall batch sizes.

Furthermore, due to the high costs linked to such pharmaceutical products and/or therapeutics, in particular with respect to the manufacturing itself and/or with respect to the intellectual property connected thereto, and also due to maintaining a high quality level, there is a demand for carrying out a production of such products and/or therapeutics in a secure and/or manipulation-protected manner.

### SUMMARY OF THE INVENTION

The present invention is therefore based on the object of solving the above-mentioned problems, in particular to be able to manufacture individual and/or ultrasmall batch sizes of products in the pharmaceutical sector efficiently and in particular in a secure and/or manipulation-protected manner.

This object is achieved according to the invention by a production system for manufacturing a product, in particular a biological-pharmaceutical product, having the features of claim 1.

Advantageous refinements of the invention are described in the dependent claims.

It is accordingly provided that the production system in particular comprises multiple processing units for accommodating and/or treating an educt, wherein each processing unit comprises at least one computing unit.

It can be provided, for example, that a processing unit is designed as a patient box, in particular for accommodating and/or transporting an educt, and/or as a tool box, in particular for treating an educt, and/or as a tool unit, in particular for treating an educt, and/or as a sensor unit, in particular for analyzing an educt. It is to be understood that the preceding list is by way of example and is not exhaustive. In particular, the processing units are described in more detail in the following description.

The production system can furthermore comprise:
a, in particular suitable, clean room area;
an airlock device for feeding an object from the surroundings of the production system into the, in particular suitable, clean room area and/or for removing an object from the clean room area;
a handling device for moving the object within the clean room area; and
a storage device arranged inside the clean room area, which comprises multiple storage spaces for accommodating multiple objects.

It can be provided that one or more processing units are designed as the handling device.

It can be provided that one or more processing units are designed as part of the storage device.

An object, in particular the object from the surroundings of the production system for feeding into the clean room area and/or the object for removal from the clean room area, can comprise at least one of the following:
an educt, in particular a material comprising cells, for example a starting material comprising cells, and/or a biological-pharmaceutical material and/or a biological-pharmaceutical workpiece,
an intermediate product, in particular an intermediate product comprising cells and/or a biological-pharmaceutical intermediate product, a tool or a tool unit, in particular an educt-touching and/or product-touching tool or tool unit and/or in particular a non-educt-touching and/or non-product-touching tool or tool unit,
one or more consumable materials,
a product, for example the product to be manufactured, in particular a biological-pharmaceutical product, for example the biological-pharmaceutical product to be manufactured.

It is to be understood that the preceding list is by way of example and is not exhaustive. Alternatively or additionally, it can be provided that the object comprises one or more and/or a combination of the above-described listed elements.

The educt can comprise cells and/or a biological material in particular distinguished from cells.

An educt can be a patient-related educt and/or a pooled educt made up of multiple patient-related educts.

The educt can be at least partially liquid, and/or at least partially semisolid, and/or at least partially solid. Alternatively, the educt can be completely liquid, or completely semisolid, or completely solid. For example, the cells of the educt can be provided, for example can be formed in particular as a cell suspension, for example as cells in a suspension culture, or as adherent cells, for example as cells on a culture surface in particular of a container or educt container, for example of a culture vessel and/or a bag.

The cells can be at least partially fresh and/or can be at least partially in a frozen state, due to which in a certain sense the educt can be present, for example, in a liquid, a semisolid, and/or a solid state.

It can be provided that the educt is arranged in a container or educt container, in particular a vessel, for example a culture vessel, a cell culture vessel, a bottle, a vial, a bag, a cassette (for example, a cassette made of a hard plastic, which is biocompatible in particular), and/or a titration plate (for example, a micro-titration plate). It can be provided that the container or educt container, including its above-mentioned examples, in each case in a suitable, in particular biocompatible manner, is partially or completely coated and/or is partially or completely uncoated.

The product can be a biological-pharmaceutical product for a personalized therapy. In a certain sense, the biological-pharmaceutical product can be a personalized therapeutic. It can be provided that a or the personalization is intended for a patient or a patient group, in other words for one or more patients. The biological-pharmaceutical product can be, for example, a cell therapeutic, for example an autologous and/or an allogenic cell therapeutic, or a gene therapeutic, for example an autologous and/or an allogenic gene therapeutic.

The clean room area can define an aseptic production area.

In particular, the clean room area can be designed and/or classified as described in the application DE 10 2021 207 738.2, to which reference is hereby made.

The production system can comprise a system for setting the ambient properties of the clean room area, wherein, for example, the system for setting the ambient properties of the clean room area can in particular comprise an air device, by means of which an air pressure, in particular a positive pressure and/or a negative pressure, is settable in the clean room area.

It can be provided that one or more processing units are designed as the system for setting the ambient properties of the clean room area and/or in particular as the air device.

The production system can comprise a housing, in particular for separating/delimiting the clean room area toward the surroundings of the production system and obtaining its clean room class, wherein the housing surrounds the clean room area, in particular the clean room area and the airlock device.

It can be provided that the housing in a certain sense surrounds the entire production system.

The airlock device can be configured to feed an object from the surroundings of the production system via the or through the housing into the clean room area and/or to remove an object from the clean room area via the or through the housing.

It can be provided that the airlock device comprises at least one entry airlock and at least one exit airlock. This can be used in particular for a directed object guidance or a directed object flow. Additionally or alternatively, it can be provided that the entry airlocks and the exit airlocks are categorizable with respect to an object to be passed through them and can accordingly be provided as part of the airlock device. Optionally, the airlock device can comprise one or more maintenance airlocks, via which the clean room area is accessible from the surroundings of the production system, in particular for human operators or users.

Additionally or alternatively, it can be provided that the clean room area comprises multiple clean room subareas. In other words, the clean room area can be divided into multiple clean room subareas. For example, it can be conceivable that the handling device and the storage device are arranged in a common clean room subarea or in different clean room subareas.

In particular, the clean room subareas can be designed and/or classified as described in the application DE 10 2021 207 738.2, to which reference is hereby made.

The production system can comprise one or more (electric) power sources and/or one or more interfaces connectable to one or more (electric) power sources, which are arranged inside the clean room area, in particular the housing, and are configured to supply at least one of the components comprised by the production system or a component part of the production system with electric current, which (component) or which (component part) is associated with the one or more power sources, for example via the one or more interfaces.

It can be provided that one or more of the processing units are designed as workpiece carriers, which are used to accommodate objects, among other things educts and/or tools and/or consumable materials.

In particular, it is conceivable that a workpiece carrier is designed as said patient box and/or said tool box. Additionally or alternatively, it can be provided that the production system comprises multiple patient boxes and/or multiple tool boxes each for accommodating one or more workpiece carriers including educts arranged and/or accommodated thereon.

It can be provided that each workpiece carrier comprises a means for fixing, in particular a means for formfitting and/or friction-locked and/or magnetic fixing, for a respective accommodation of educts and/or tools and/or consumable materials, by which the educts and/or the tools and/or the consumable materials are held reliably.

The workpiece carriers can remain permanently, in particular over one or more complete production processes, within the clean room area.

The production system can comprise multiple types of workpiece carriers, which differ from one another with respect to the shape and/or dimensioning of a receptacle area of the respective workpiece carrier.

One type of workpiece carrier can be used to accommodate a vessel, for example a culture vessel, for accommodating an educt.

This can be in particular the workpiece carrier configured as a patient box, in a certain sense the patient box.

One type of workpiece carrier can be used to accommodate one or more tool units and/or tool consumable means for carrying out a treatment procedure on an educt and/or for carrying out a maintenance procedure within the clean room area.

This can be in particular the workpiece carrier configured as a tool box, in a certain sense the tool box.

One type of workpiece carrier can be used to accommodate consumable materials to feed them to an educt and/or to carry out a treatment procedure on an educt.

This can be in particular the workpiece carrier configured as a tool box, in a certain sense the tool box.

One or more workpiece carriers can each comprise one or more action units for carrying out an action on an educt and/or on a tool.

This can be in particular the workpiece carrier configured as a patient box and/or tool box, in a certain sense the patient box and/or tool box.

One or more action units can be designed as a treatment unit, for example as a tool unit, for carrying out a treatment procedure on the educt.

It can be provided that a processing unit is designed as an action unit and/or treatment unit and/or tool unit. In a certain sense, a processing unit can therefore optionally comprise a processing unit, in particular a further processing unit.

It can be provided that a treatment unit, such as a tool unit, is configured to carry out a rocking and/or a tilting and/or a shaking and/or a rotating movement, in particular by means of at least one correspondingly associated actuator unit or actuator system unit, so that this type of movement, for example this type of movement combination, is transferable to the educt.

It can be provided that a treatment unit, for example a tool unit or a component of a tool unit, is configured to control the temperature of the workpiece carrier, in particular to heat and/or cool it.

One or more action units can be designed as a sensor unit for determining a current value of a parameter of the respective workpiece carrier and/or an educt arranged thereon and/or a tool arranged thereon and/or consumable materials arranged thereon.

A parameter can in particular be one of the following: a temperature, an ambient humidity, an ambient pressure, a gas mixture composition, an orientation, for example an angular orientation, an oscillation, an occupancy, in particular an occupancy status, for example a storage occupancy status, and a positioning, for example a locked positioning or a non-locked positioning and/or a location positioning, of the workpiece carrier and/or of an object accommodated by means of the workpiece carrier. It is to be understood that the preceding list is by way of example and is not exhaustive. Alternatively or additionally, it can be provided that the parameter comprises a combination of the above-described listed elements.

One or more workpiece carriers can comprise a storage device for storing energy and/or one or more consumable materials.

The workpiece carriers can be reusable multiple times, in particular the storage device can be electrically rechargeable and/or refillable with consumable material.

The workpiece carriers can comprise at least one interface, by means of which the electric energy and/or the consumable material is rechargeable or refillable and dispensable (again).

The production system can comprise a charging station arranged inside the clean room area for charging the storage device and/or a refilling station arranged inside the clean room area for refilling the storage device.

It can be provided that the refilling station is designed as a stationary refilling station, to which consumable material can be fed via the airlock device in order to fill it, and/or that the refilling station is designed as a locally mobile refilling station, which, in particular in a state filled with consumable material, can be fed via the airlock device from the surroundings of the production system into the clean room area and, in particular in a state emptied of consumable material, can be removed through the airlock device from the clean room area. It can furthermore be provided that the refilling station is in particular arrangeable, for example is arranged, on a storage space of the storage device.

One or more workpiece carriers can comprise one or more coupling devices, by means of which multiple, in particular two or more, workpiece carriers are connectable to one another.

In other words, one or more processing units can comprise said coupling devices for connecting processing units.

The storage device can comprise a storage transport device for transporting objects toward the storage spaces and/or away from the storage spaces.

It can be provided that one or more processing units are designed as the storage transport device.

The handling device can comprise a distribution unit, via which the storage transport device can be fed objects to be transported and/or these can be received from it. Optionally, the distribution unit can be designed as a or as part of an airlock transport device, or can be associated with such a device.

It can be provided that the distribution unit is designed as a processing unit separate from the handling device.

In other words, one or more processing units can be designed as the handling device and at least one further processing unit can be designed as the distribution unit.

The storage transport device can be a device different from the handling device.

The storage transport device can comprise a rail-guided transport system and/or one or more freely moving transport vehicles.

It can be provided that one or more processing units are designed as the rail-guided transport system and/or that a processing unit is designed as a freely moving transport vehicle.

The storage transport device can comprise one or more rail-guided storage transport units, by means of which one or more storage racks of the storage device are accessible in particular for storage and removal procedures and for treatment procedures and/or maintenance procedures.

The storage transport device can comprise one or more storage transport units designed as gantry conveyors, wherein in particular by means of the one or more gantry conveyors, one or more storage racks of the storage device are accessible in particular for storage and removal procedures and for treatment procedures and/or maintenance procedures.

The storage transport device, in particular the rail-guided transport system, the one or the multiple freely moving transport vehicles, the one or the multiple rail-guided storage transport units, and/or the one or the multiple gantry conveyors can be guided by induction loops and/or magnetically guided, in particular guided by permanent magnets and/or guided by solenoid coils. Additionally or alternatively, a sensor-based guidance, in particular a camera-based guidance and/or a radar-based/a lidar-based guidance, can be provided.

Additionally or alternatively, it can be provided that the storage transport device comprises one or more elevator devices, by means of which one or more storage space surfaces of the storage device are accessible in particular for storage and removal procedures and for treatment procedures and/or maintenance procedures.

The handling device can be or comprise a multiaxis robot arm. Additionally or alternatively, it can be provided that the handling device is or comprises a pick and place robot or a cable robot, and/or that the handling device is or comprises a suitable gripper actuator system configured on a planar runner. Additionally or alternatively, it can be provided that the rail-guided transport system and/or the one or the multiple freely moving transport vehicles of the storage transport device respectively comprise at least one multiaxis robot arm.

The storage transport device and/or the handling device can be associated with a changing device of the production system, by means of which different gripper units and/or carrier units are installable on one or more storage transport units of the storage transport device and/or on one or more handling units of the handling device, in particular to carry out different storage procedures, transport procedures, handling procedures, maintenance procedures, and/or treatment procedures by means of the respective storage transport unit and/or handling unit.

It can be provided that a processing unit is designed as said changing device.

The storage transport device and/or the handling device can be associated with a cleaning device of the production system, by means of which at least one gripping unit and/or carrier unit of one or more storage transport units of the storage transport device and/or one or more handling units of the handling device can be cleaned, in particular after carrying out a storage procedure, transport procedure, handling procedure, maintenance procedure, and/or treatment procedure.

It can be provided that a processing unit is designed as said cleaning device.

It can be provided that the cleaning device is configured for cleaning in particular with application of irradiation and/or gassing.

The cleaning device of the production system associated with the storage transport device and/or the handling device can in particular be arranged in or on the airlock device or adjacent, for example directly adjacent, to the airlock device within the clean room area.

It can be provided that the cleaning device of the production system associated with the storage transport device and/or the handling device is spatially separated from the airlock device inside the clean room area.

By means of the handling device, one or more objects, for example as described above, can be transferable from an airlock space of the airlock device to a storage transport device, in particular to one or more storage transport units of a storage transport device.

The airlock device can comprise an entry airlock, via which an airlock space, in particular an entry airlock space, is accessible from the surroundings and which is configured for feeding an object from the surroundings of the production system into the clean room area, and an exit airlock, via which an airlock space, in particular an exit airlock space, is accessible from the surroundings and which is configured for removing an object from the clean room area.

The handling device can comprise an airlock transport device, by means of which one or more objects are transportable from an airlock space, in particular an entry airlock space, to a further airlock space, in particular a transfer airlock space, and/or from an airlock space, in particular a transfer airlock space, to a further airlock space, in particular an exit airlock space.

It can be provided that the handling device, in particular the airlock transport device, comprises a displacement unit, by means of which one or more objects are movable between the various airlock spaces.

It can be provided that one or more processing units are designed as the airlock device, in particular the airlock transport device, and/or at least one processing unit is designed as said displacement unit.

Additionally or alternatively to at least one airlock space, in particular the entry airlock space and/or the exit airlock space, an unpacking space and/or a packing space can be provided in the airlock device, wherein optionally: the entry airlock space can be associated with the unpacking space, arranged adjacent or spatially separated, or the entry airlock space can be the unpacking space, and/or the exit airlock space can be associated with the packing space, arranged adjacent or spatially separated, or the exit airlock space can be the packing space.

The storage transport device and/or the handling device can each comprise one or more workpiece carrier receptacles for accommodating and transporting one or more workpiece carriers, in particular one or more of the above-described workpiece carriers.

It can be provided that, by means of the handling device, one or more workpiece carriers, in particular one or more equipped workpiece carriers (for example, equipped with an educt), are transferable from an airlock space of the airlock device to the storage transport device, in particular to the one or the multiple storage transport units of the storage transport device, in particular via the one or the multiple workpiece carrier receptacles.

The airlock device can comprise a cleaning device for cleaning one or more workpiece carriers and/or for cleaning one or more workpiece carrier receptacles.

The production system can comprise a cleaning device, to which workpiece carriers can be fed for the cleaning thereof, in particular after completed transport of an object by means of the respective workpiece carrier.

It can be provided that the cleaning device associated with the storage transport device and/or the handling device, the cleaning device of the airlock device, and the cleaning device of the production system form a common cleaning device of the production system.

It can be provided that each of the respective cleaning devices is formed at least partially or completely separately from one another, in particular form independent devices. Each of these cleaning devices can be designed as a processing unit or can be formed by such a processing unit.

The storage device can comprise one or more storage racks and/or one or more storage space surfaces, each of which comprises multiple storage spaces.

It can be provided that the storage spaces can be temperature controlled, in particular can be individually temperature controlled, by means of an in particular respectively assigned temperature control device of the storage device, wherein optionally one or more processing units can be designed as the temperature control device.

It can be provided that multiple storage space surfaces, in particular two or more storage space surfaces, are arranged one above another, in particular in a vertical direction. In a certain sense, the multiple storage space surfaces can be arranged like a stack.

It can be provided that multiple storage space surfaces, in particular two or more storage space surfaces, for example two or more storage space surfaces arranged one above another, are connectable or connected to one another via the storage transport device, in particular via one or more elevator devices of the storage transport device.

Educts and/or tools and/or consumable materials can be storable in the storage spaces or removable therefrom by means of the storage transport device.

In particular, educts and/or workpiece carriers and/or tools and/or consumable materials can be storable in the storage spaces or removable therefrom by means of the storage transport device of the storage device.

One or more storage spaces can be accessible from multiple directions and/or by means of multiple storage transport units of the storage transport device.

The storage spaces can be accessible in the vertical direction from above or in the horizontal direction laterally by means of the storage transport device.

Multiple rows of storage spaces aligned substantially horizontally can be arranged located one above another in the vertical direction. In a certain sense, the storage spaces can be arranged in the form of a matrix.

A plurality of the storage spaces, in particular all storage spaces, can be rack spaces of one or more storage racks.

The storage device can comprise multiple types of storage spaces which are different from one another for accommodating different types of workpiece carriers, wherein optionally the different types of storage spaces can differ from one another with respect to their shape and/or dimensioning and/or with respect to their function and/or equipment.

The storage device can comprise multiple storage spaces which form treatment stations for treating educts.

It can be provided that a processing unit is designed as a storage space.

The storage device can comprise multiple storage spaces which form tool stations for keeping, for maintaining, for cleaning, for changing, for electrically charging, and/or for monitoring tools.

The storage device can comprise multiple storage spaces which comprise one or more interfaces, in particular for an (electric) power supply and/or for a signal transmission, for example a control and/or regulating signal transmission, which are connectable to one or more corresponding interfaces of an object that can be accommodated in the storage spaces, in particular processing units, for example workpiece carriers, preferably patient boxes or tool boxes.

The workpiece carriers already described can be designed in particular as processing units, for example as patient boxes or tool boxes, for accommodating and/or treating an educt.

It is to be understood that statements related to workpiece carriers can also relate to patient boxes and/or tool boxes.

A patient box and/or a tool box can define a processing cell, in particular an aseptic processing cell. In a certain sense, a processing unit designed as a patient box and/or tool box can be a processing cell, in particular an aseptic processing cell.

A patient box and/or a tool box, in particular the tool box, can comprise interfaces for one or more tool units, in particular for gassing and/or temperature control.

Additionally or alternatively, it can be provided for this purpose that a patient box and/or a tool box, in particular the tool box, is equipped with a means for formfitting and/or friction-locked fixing.

The patient boxes and/or the tool boxes can be storable in storage spaces of the storage device, in particular fully automatically by means of a storage transport device of the storage device, for example the one already described.

One or more of the patient boxes and/or the tool boxes can surround a closable interior, wherein in particular an aseptic production area can optionally be definable by the closable interior. In a certain sense, it can be provided that the closable interior is an aseptic production area.

The patient boxes and/or the tool boxes can be openable and/or closable independently and/or by means of the handling device, in particular to introduce an educt and/or to remove a product produced from an educt and/or to carry out a treatment procedure.

One or more of the patient boxes and/or the tool boxes can each comprise one or more conditioning units, which are integrated or detachably arranged thereon or detachably arrangeable thereon, by means of which a temperature and/or a pressure and/or a humidity and/or a gas composition in the interior of the respective box can be influenced, in particular can be controlled and/or regulated.

One or more of the conditioning units can be tool units of a tool system of the production system, which are arrangeable as needed on the respective patient box and/or the respective tool box or removable therefrom, in particular fully automatically by means of a storage transport device of the storage device, for example the one already described, and/or by means of the handling device.

Additionally or alternatively, it can be provided that one or more of the conditioning units are tool units of a tool system of the production system which are arrangeable as needed on the respective patient box and/or the respective tool box or removable therefrom, in particular independently by means of a separate actuator system and/or using an actuator system configured in or on the respective patient box and/or the respective tool box.

The patient boxes and/or the tool boxes can comprise one or more connection elements, which are connectable to connection elements corresponding thereto on one or more treatment spaces, in particular to supply the respective patient box and/or the respective tool box with electric energy and/or consumable substances, in particular liquids and/or gases.

The storage device can comprise storage spaces provided with connection elements, so that one or more treatment procedures for treating the educts can be carried out at storage spaces of the storage device used as treatment spaces.

The storage spaces of the storage device and/or the patient boxes and/or the tool boxes can comprise fixing elements, by means of which the patient boxes and/or the tool boxes are positionable and/or fixable at the storage spaces.

The patient boxes and/or the tool boxes, in particular the tool boxes, can each comprise one or more tool receptacles for accommodating and securing one or more tools, in particular in the interior of the respective box, wherein in particular the one or the multiple tool receptacles can each comprise one or more connection elements, which are connectable to connection elements corresponding thereto on the respective tool to be accommodated, in particular to supply the respective tool with electric energy and/or consumable substances, in particular liquids and/or gases, and/or to establish a signaling coupling of the respective tool with the respective box.

The patient boxes and/or the tool boxes can also be (respectively) storable at storage spaces in a state not used for product manufacturing, in particular after a cleaning by means of a cleaning device of the production system, for example the one already described.

The patient boxes and/or tool boxes stored at storage spaces in a state not used for product manufacturing can be removable from the storage spaces to accommodate or introduce educts, in particular fully automatically by means of the storage transport device of the storage device.

Additionally or alternatively, it can be provided that a patient box and/or a tool box comprises one or more connection elements, which are connectable to connection elements corresponding thereto on a further patient box and/or tool box, by which an assembled patient box and/or an assembled tool box and/or a combination thereof can be provided.

For a possible design and/or configuration of the assembled boxes, reference is made to the remaining description with respect to the patient boxes and/or the tool boxes. It is to be understood that one or more storage spaces can be configured as storage spaces for assembled boxes.

The production system can comprise an unpacking device for unpacking objects to be fed to the clean room area and/or a packing device for packing objects to be removed from the clean room area, for example the manufactured product.

The unpacking device and/or the packing device can be integrated in the airlock device or form a component part thereof.

The unpacking device and/or the packing device can be designed as processing units or can be formed by such processing units.

The airlock device can comprise an entry airlock space, which is accessible from the surroundings of the production system and at which in particular a packed object is arrangeable by a person and/or in an automated manner, and/or an exit airlock space, which is accessible from the surroundings of the production system and at which in particular a packed object is removable by a person and/or in an automated manner.

The packed object can be transportable by means of an airlock transport device of the production system from the entry airlock space to an unpacking space, for example the one already described, and can be unpackable in an automated manner at the unpacking space or the packed object can be unpackable in an automated manner at the entry airlock space, and/or the object to be packed can be transportable by means of an airlock transport device of the production system from a transfer airlock space to a packing space and can be packable in an automated manner at the packing space or the object to be packed can be packable in an automated manner at the exit airlock space.

The airlock device can comprise a cleaning device, for example the one already described, for cleaning a packing of the packed object and/or the unpacked object.

The packing of the packed object, in particular the packed educt, can be dischargeable in a sterile manner by means of the unpacking device after the unpacking, for example via the exit airlock space and/or via a waste airlock space of a separately designed waste airlock of the unpacking device or the airlock device, due to which the unpacked object, in particular the unpacked educt, cannot come into contact with the packing.

It can be provided that the packing device and the unpacking device are formed in a common device for unpacking and/or packing.

As was already described in conjunction with the conditioning units of the patient boxes and/or the tool boxes, the production system can comprise a tool system, by means of which in particular treatment procedures on educts and/or maintenance procedures within the clean room area can be carried out.

The tool system can comprise multiple in particular fully automatic tool units for carrying out different treatment and/or maintenance procedures.

It can be provided that a processing unit is designed as a tool unit. Additionally or alternatively, one or more of the tool units can be associated with a tool box and/or can be comprised by such a tool box.

It can be provided that a tool unit described herein is a tool described herein and/or a tool described herein is a tool unit described herein.

The tool system can comprise one or more tool units designed as feed units for feeding consumable materials to the educts and/or one or more tool units designed as removal units for removing a sample from an educt.

One or more of the tool units of the tool system can each comprise one or more multiuse components and/or one or more single-use components.

The tool system can comprise a fitting unit, by means of which, fully automatically, one or more single-use components of a tool unit are removable after a usage thereof, in particular disposable, and are replaceable by one or more new single-use components.

The fitting unit can comprise a handling unit, in particular a multiaccess robot arm, by means of which a fully automatic fitting procedure can be carried out.

It can be provided that a cleaning can be carried out before, during, in particular between a stripping procedure and an equipping procedure, and/or after a fitting procedure by means of a cleaning unit of the fitting unit.

The tool system can comprise multiple portable tool units, which can be fed to a treatment space for treating an educt to carry out a treatment procedure, which treatment space can in particular be a storage space for storing the educt at the same time, and can be temporarily stored there.

One or more portable tool units of the tool system can be able to be fed by means of a storage transport device, in particular one or more storage transport units, of the production system to a tool storage space and/or to a treatment space and/or to a fitting space.

One or more tool units can be designed and configured to carry out a treatment procedure and/or a maintenance procedure by means of the handling device.

One or more tool units can be designed and configured to carry out a treatment procedure and/or a maintenance procedure independently of the handling device, in particular automatically.

A tool unit can be able to be parked, temporarily stored, or stored in a storage space, and/or connected to associated interfaces in the storage space.

A tool unit can be a tool for feeding and/or discharging, for example suctioning off a liquid, in particular a tool for pumping a liquid, for example a pumping tool, wherein the tool unit can be configured in particular to feed and/or discharge a predetermined amount of liquid, in particular for sample taking for analytics.

A tool unit can be a tool for mixing at least one liquid, in particular at least one liquid with at least one liquid, gaseous, and/or solid substance. For example, the tool unit can be a pump for circulating.

A tool unit can be a means for biochemical manipulation of cells, by means of which at least one substance can be fed to an educt, and/or a temperature of an educt is changeable, and/or an ambient pressure of an educt is changeable, wherein the educt is arranged, in particular accommodated in particular in a tool carrier, in particular in a receptacle box. In this way, in particular the surroundings of the educt, in particular the cells of the educt, can be changeable to thus change the educt itself, in particular the cells of the educt itself, in particular with regard to a cell growth and/or a cell surface, for example a cell membrane surface, and/or a cell metabolism.

A tool unit can be a means for concentrating and/or separating liquids, in particular by means of a column, for example a separating column, and/or a centrifuge.

A tool unit can be a means for analyzing process parameters by means of a probe and/or a sensor, wherein the probe and/or the sensor is configured, for example, to register, detect, and/or determine at least one of the following: at least one temperature, at least one particle, for example at least one particle concentration, at least one oscillation, at least one acceleration, at least one cell number, at least one cell density, at least one turbidity, for example at least one light absorption due to a liquid turbidity, at least one gas composition, at least one gas, for example CO2, and/or at least one chemical substance, in particular at least one biochemical substance, for example at least one metabolite.

It is to be understood that the preceding list is by way of example and is not exhaustive.

It can be provided that the tool system is in particular configured fully automatically.

In a certain sense, the production system can in particular comprise a fully automatic tool system, which comprises one or more tool units for carrying out treatment procedures on an educt, wherein the tool system is arranged inside the clean room area, for example inside a clean room subarea, for example, inside a patient box and/or a tool box as a clean room subarea.

It can be provided that a supply apparatus for supplying the tool system with consumable materials is associated with the tool system. In a certain sense, the production system can comprise a tool system, for example the one described above, and a supply device for supplying the tool system with consumable materials, wherein in particular the supply device can comprise or use one or more storage spaces of the storage device arranged inside the clean room area for storing consumable materials.

Consumable materials to be used in a treatment procedure can be able to be provided, in particular before their use, in the storage spaces and, optionally, can be prepared for use, in particular can be temperature controlled.

The supply device can comprise one or more disposal units, by means of which in particular used, consumed, and/or contaminated consumable materials and/or tool consumable means are removable from the clean room area.

One or more disposal units can be associated with the fitting unit of the tool system, in particular can be handled thereby, and/or can form a component part of the fitting unit of the tool system.

The supply device can comprise a refilling device arranged inside the clean room area, by means of which in particular consumable substances present in liquid form can be fed to one or more storage devices of one or more tool carriers and/or one or more storage spaces.

Additionally or alternatively, the refilling device can be designed as the above-described refilling station and/or can be associated therewith.

As already described above, one or more sensor units, in particular probes, sensors, and/or substantially similar measuring elements can be comprised in the production system.

In a certain sense, the production system can comprise one or more sensor units for determining one or more values of one or more parameters of one or more educts, and/or one or more tool carriers, and/or one or more tools, and/or one or more consumable materials.

The determination can be able to be carried out in an at least partially automated manner, in particular fully automatically, by means of one or more sensor units.

An occupancy status of one or more storage spaces of a storage device of the production system can be able to be determined, in particular monitored, by means of one or more sensor units.

A local temperature and/or a local humidity and/or a local illuminance or irradiance at or in one or more storage spaces of the storage device of the production system can be able to be determined, in particular monitored, by means of one or more sensor units.

The production system can comprise one or more sensor units for determining a treatment progress during the treatment of an educt or between two treatment procedures, for example on the basis of at least one fluid of the educt.

One or more sensor units for determining the treatment progress can be arranged on or in each of one or more processing units, in particular patient boxes and/or tool boxes and/or storage spaces, in particular integrated therein, to accommodate educts.

In particular, the sensor units can be designed and/or configured as described in the application DE 10 2021 207 738.2, to which reference is hereby made.

In other words, this can be summarized to state that the production system comprises one or more sensor units, by means of which one or more, essentially all processing steps, in particular one or more, essentially all treatment procedures, and/or one or more, essentially all process statuses, in particular one or more, essentially all treatment statuses can be monitored and, in particular if needed, regulated, in particular readjusted, and/or controlled, in particular tracked.

The sensor units can in particular address: at least one temperature, at least one particle, for example at least one particle concentration, at least one oscillation, at least one acceleration, at least one cell number, at least one cell density, at least one turbidity, at least one turbidity of an educt and/or product suspension, for example at least one light absorption due to a liquid turbidity in particular in an educt and/or product suspension, at least one gas composition, at least one gas, for example CO2, and/or at least one chemical substance, in particular at least one biochemical substance, for example at least one metabolite, for example glucose.

The above-described sensor units can be arrangeable in the production system in the clean room area as needed in particular in relation to one or more objects, for example educts and/or tools and/or storage spaces and/or consumable materials and/or processing units, such as patient boxes and/or tool boxes.

As described above, the production system can comprise one or more cleaning devices.

The cleaning device can be designed as a separate device in the clean room area and/or can be a component of the tool system, in particular a tool or a tool unit, and/or of the airlock device and/or the storage device.

Additionally or alternatively, the cleaning device can comprise one or more autoclaving devices, by means of which one or more objects inside the clean room area are sterilizable.

It can be provided that the one or more autoclaving devices are implemented as an autoclaving device described in conjunction with the unpacking device and/or with the packing device.

The cleaning device can comprise a cleaning station, which is stationary in particular and by means of which at least one processing unit, in particular the at least one workpiece carrier, in particular at least one patient box and/or at least one tool box can be cleaned, in particular can be disinfected and/or sterilized.

The cleaning station can comprise a washing machine and/or an autoclaving device.

It can be provided that the production system comprises at least one control unit, by means of which and/or via which the production system can be operated, in particular regulated and/or controlled.

In a certain sense, it can be provided that the production system comprises at least one control unit, which is designed and programmed to at least partially or completely regulate and/or control the production system.

Additionally or alternatively, it can be provided that the at least one control unit is designed and programmed to regulate and/or control at least one of the components comprised by the production system or a component part of the production system, with which component or component part the at least one control unit is/are associated.

It can be provided that the at least one control unit is formed by one or more, in particular all, computing units of the processing units.

In a certain sense, the one or more, in particular all, computing units of the processing units can form the at least one control unit.

Additionally or alternatively, it can be provided that the at least one control unit is designed and programmed as a mediator unit between a computer system which is associated or can be associated with the production system and the production system itself, wherein said computer system is designed and programmed to at least partially or completely regulate and/or control the production system.

The computer system which is associated or can be associated with the production system can be arranged in the surroundings of the production system and/or can be arranged spatially separated or apart and can be connectable or connected to the production system, in particular to the at least one control unit designed as a mediator unit, in particular in a wireless and/or wired manner, for example by means of one or more communication interfaces of the production system.

It can be provided that the production system comprises the computer system which is associated or can be associated with the production system.

Additionally or alternatively, it can be provided that the computer system which is associated or can be associated with the production system is a control computer system, in particular a cloud-based control computer system and/or a control server.

Additionally or alternatively, it can be provided that the at least one control unit is designed as at least one independent control unit.

The at least one independent control unit can be arranged in the surroundings of the production system and/or can be arranged spatially separated or apart and can be connectable or connected to the remainder of the production system, in particular in a wireless and/or wired manner, for example by means of one or more communication interfaces of the production system.

Additionally or alternatively, the at least one independent control unit can be arranged inside the clean room area, in particular the housing, or at the production system.

In a certain since, it can be provided that the production system can be operated, in particular can be regulated and/or controlled, by means of said computing units of the processing units and/or by means of at least one, in particular independent control unit and/or by means of said computer system which is associated or can be associated with the production system. It is to be understood that said components can be configured, alone or in any combination thereof, for operating, in particular for regulating and/or controlling, the production system.

Additionally or alternatively, it can be provided that the at least one control unit is programmed and designed to identify, code, and/or associate one or more objects in the production system, in particular by means of a respective ID associated with an object, wherein, optionally, the ID can be associated with a respective object in particular by means of RFID means and/or barcode means and/or as unique information, in particular a digital fingerprint.

The production system can comprise one or more communication interfaces and/or one or more human-machine interfaces, abbreviated: HMI interfaces, by means of which one or more items of information about the production system and/or at least one component of the production system and/or at least one part of the production system can be provided, in particular to at least one user and/or one or more computer systems, for example with regard to the production system falling below a predetermined amount of consumable materials.

The at least one control unit can be designed and programmed to be able to communicate with one or more communication interfaces and/or one or more human-machine interfaces, abbreviated: HMI interfaces, of the production system.

The at least one control unit can be designed and programmed to provide information to a human operator or user via the communication interfaces and/or via the HMI interfaces, for example interference information and/or information about a status of a production of a product and/or information about storage stocks of the storage device.

It can be provided that the at least one control unit is designed and programmed to associate a or the educt of one of the multiple processing units on the basis of one of multiple production programs to manufacture one of multiple products that can be manufactured by means of the production system.

In other words, the production system can comprise the multiple processing units for accommodating and/or for treating an educt, which are configured as described above and/or below and which respectively comprise at least one computing unit, and the production system can furthermore comprise the at least one control unit, which is designed and programmed to associate the educt of one of the multiple processing units on the basis of one of multiple production programs for manufacturing one of multiple products that can be manufactured by means of the production system.

A production program can comprise multiple, in particular encrypted production program packets, wherein each production program packet contains at least one item of information which refers to another production program packet and/or comprises instructions for accommodating and/or treating an educt.

A production program can represent a formula for manufacturing a product, in particular a personalized product based on patient-related information.

A defined sequence of the multiple production program packets can form the production program for manufacturing a product that can be manufactured by means of the production system.

In particular, it can be provided that the complete sequence is only determinable from all references with respect to the other production program packets.

It can be conceivable, for example, that said defined sequence can be definable, in particular dynamically definable, by the production system, in particular by the at least one control unit of the production system and/or a computer system which is associated or can be associated with the production system.

One or more production program packets can in particular be executable or executed fully automatically, until a product to be manufactured from the educt is finished, in particular in consideration of a quality analysis which is carried out or can be carried out, in particular by means of one or more sensor units.

The product thus manufactured can be automatically transportable or transported to the airlock device for the removal thereof from the production system. This can be comprised, for example, as an instruction by a production program packet.

The product can be filled and/or packed before the removal thereof, in particular filled and/or packed ready for use. This can be comprised, for example, as an instruction by a production program packet.

In particular, a production program packet can comprise instructions for one or more treatment procedures, for example instructions for:
feeding a liquid and/or a gas, in particular a gas mixture,
discharging, in particular pumping out, a liquid and/or a gas, in particular a gas mixture,
discharging, in particular pumping out, a part of a liquid and/or a gas, in particular a gas mixture, for an analysis by means of one or more sensor units,
mixing one or more components, in particular at least one liquid with at least one further component, for example a further liquid,
concentrating and/or separating a liquid in particular by means of a column, for example a separating column, and/or a centrifuge,
analyzing an educt and/or an intermediate product and/or a product in particular by means of one or more sensor units,
storing, in particular placing in and/or removing from storage, one or more objects in particular by means of the storage transport device,
controlling the temperature of, in particular incubating, one or more objects, in particular one or more educts.

It is to be understood that the preceding list of the treatment procedures or their instructions of production program packets is by way of example and is not exhaustive. Alternatively or additionally, it can be provided that one or more treatment procedures are combinable with one or more treatment procedures in a single treatment procedure and/or in any combination thereof.

It can be provided that one or more, in particular all production program packets are encrypted and/or can be provided in encrypted form, in particular at one or more of the processing units.

A processing unit, in particular its at least one computing unit, can be designed and programmed to execute instructions of at least one production program packet, in particular to decrypt and then execute them.

A plurality of the processing units can form a group of processing units, which (group) is based on a sequential order of the processing units among one another with regard to the references of the production program packets and by means of which (group) a product can be manufactured.

It is to be understood that the composition of said group can first result from all references of the production program packets and therefore a complete group can first be completely clear with completion of a manufacture of a product.

Additionally or alternatively, it can be provided that a plurality of the processing units can form a group of processing units which executes a production program packet, in particular only one production program packet, in particular decrypts and then executes it, to ensure a success of a treatment procedure contained in the production program packet and/or to ensure safety from failure.

It can be provided that one or more, in particular all, of the production program packets are each provided on one processing unit, in particular on its at least one computing unit, and/or are each transmittable to one processing unit, in particular to its at least one computing unit, in particular by means of the at least one control unit and/or by means of a computer system which is associated or can be associated with the production system.

It is to be understood that one or more, in particular all of the production program packets can be storable in a dynamically manageable and/or changeable storage pool, which can be operable and/or can be storable in particular by the at least one control unit and/or a computer system which is associated or can be associated with the production system, by which new production program packets can in particular be dynamically supplemented and/or changed there and in particular new formulas for manufacturing a product can be prepared efficiently and rapidly.

It can be provided that the production system comprises the computer system which is associated or can be associated with the production system.

Additionally or alternatively, it can be provided that the computer system which is associated or can be associated with the production system is a cloud-based computer system and/or a server.

Each processing unit, in particular each computing unit of a processing unit, can comprise at least one storage unit for providing and/or storing data, in particular one or more production program packets and/or one or more items of information and/or one or more cryptographic keys and/or an in particular decentralized database.

It is to be understood that the production system, in particular its components, for example, the above-mentioned processing units, can comprise multiple storage units and/or the production system can be associated with one or more, in particular decentralized storage units, for example, located in a cloud and/or on a server, wherein a computer program product is storable or stored on at least one storage unit, which computer program product comprises commands which, upon the execution of the computer program product by a or the above-described at least one control unit of the production system, prompts the above-described production system to be operable as described in this description. The computer program product can additionally or alternatively be stored on a computer-readable (further) storage medium.

It can be provided that the multiple, in particular encrypted production program packets comprise a starting packet and one or more following packets, wherein each packet contains at least one item of information which refers to a following packet and/or comprises instructions for accommodating and/or treating an educt.

In a certain sense, each defined sequence of the multiple production program packets which form the production program for manufacturing a product that can be manufactured by means of the production system can begin with a starting packet.

It can be provided that the educt can only be associated with a processing unit which is associated with the starting packet and/or which refers to the starting packet and/or on which the starting packet is stored, or can be associated with a processing unit which the starting packet can be provided with after or with an association of the educt.

It is to be understood that at least one of the following packets can be designed as an in particular last following packet and/or as a final packet, which contains at least one item of information which comprises instructions for delivering and/or completing a treatment of an educt and/or for finishing a product manufactured from the educt.

It is to be understood that a production program packet can also contain two or more items of information which each refer to another production program packet, for example two or more production program packets, and/or comprise instructions for accommodating and/or for treating an educt.

A processing unit, in particular its at least one computing unit, can be designed and programmed to store one or more cryptographic keys, by means of which production program packets of the production program can each be decrypted.

Such a cryptographic key can in particular be a private cryptographic key in particular for a public-key encryption.

In other words, a cryptographic key can be uniquely associated with a production program packet.

In particular, a cryptographic key can be stored by means of at least one of the processing units, by means of which a production program packet for starting the production program can be decrypted and/or activated.

In other words, a cryptographic starting key for decrypting the starting packet can be storable on at least one of the processing units, so that a manufacture of a product can be started according to the respective defined order of the multiple production program packets.

One or more production program packets can be able to be locally decrypted in a processing unit by means of the computing unit of the processing unit.

Additionally or alternatively, one or more production program packets can be able to be decrypted outside the processing units by means of the at least one control unit and/or by means of a computer system which is associated or can be associated with the production system.

The one or the multiple cryptographic keys can be assignable to the processing unit by means of the at least one control unit and/or by means of a computer system which is associated or can be associated with the production system.

Additionally or alternatively, the one or the multiple cryptographic keys can be inseparably integrated in the processing unit, in particular its at least one computing unit.

It can be provided that a processing unit comprises a security module, in particular a trusted platform module, for providing one or more cryptographic keys, wherein in particular one or more cryptographic keys for decrypting individual production program packets of the production program are storable by means of the security module.

Such a cryptographic key can in particular be a private cryptographic key in particular for a public-key encryption.

It can be provided that a processing unit, in particular its at least one computing unit, is designed and programmed to receive release information for a use and/or a provision of the one or more cryptographic keys, and/or the one or more cryptographic keys can only be used and/or provided in a time-limited manner by the processing unit.

In other words, one or more cryptographic keys can only be provided by the processing unit so they can be used and/or provided in an unrestricted or in particular in a time-restricted manner if corresponding release information, which is described in more detail in particular in the following description, is provided to the processing unit.

It can be provided that the educt can only be associated with a processing unit and/or a treatment of the educt by means of a processing unit can only be carried out if unique information related to the processing unit, which can be provided by means of the processing unit itself, and further unique information related to the processing unit, which can be acquired by means of the at least one control unit, correspond and therefore an integrity of the relevant processing unit exists.

In other words, the educt can only be associated with a processing unit and/or a treatment of the educt by means of a processing unit can only be carried out if an integrity of the relevant processing unit is set and/or confirmed.

It can be provided that such an integrity comprises a correspondence of a setpoint status of a processing unit and an actual status of the processing unit, wherein in particular the setpoint status can relate to a starting status of the processing unit. For example, it can therefore be determinable whether a manipulation and/or a change which is not provided was performed on a processing unit.

It can be provided that at least one processing unit is configured for feeding an educt into the production system and/or for removing an educt, an at least partially treated educt, and/or a product from the production system, in particular via the airlock device.

In other words, at least one processing unit can be designed as an airlock box for passing in and/or passing out an object with respect to the production system. For example, the airlock box can be a passing-in box, a passing-out box, and/or a combined passing-in-passing-out box.

In particular, this processing unit, preferably the processing unit configured as an airlock box, can be designed and programmed to execute a start, in particular a starting packet, of a production program.

It can be provided that a patient box and/or a tool box is (in each case) an airlock box and/or is/are usable as such.

It can be provided that the production system is configured to provide one or more processing units for accommodating and/or treating an educt.

The production system can be configured to determine an in particular encrypted production program packet of a production program, in particular to manufacture a product that can be manufactured by means of the production system, and one of the processing units in order to execute this production program packet, based on a production program packet of the production program which precedes the production program packet and is in particular encrypted.

The production system can be configured to activate the determined production program packet on the determined processing unit in order to decrypt the determined production program packet and then to control the processing unit to carry out one or more treatment steps on the educt based on the decrypted production program packet.

The production system can be configured to determine a production program packet of the production program which follows the decrypted production program packet and is in particular encrypted and a following processing unit in order to execute this following production program packet, based on the decrypted production program packet.

In other words, the production system can be configured to be operable in particular using the following steps, and/or in still other words, a method can be provided for at least partially controlling a or the production system to manufacture a product, in particular a biological-pharmaceutical product, wherein the production system is configured as described above and/or below and wherein the method comprises the following:
providing one or more processing units for accommodating and/or treating an educt;
determining an in particular encrypted production program packet of a production program, in particular to manufacture a product that can be manufactured by means of the production system, and one of the processing units in order to execute this production program packet, based on a production program packet of the production program which precedes the production program packet and is in particular encrypted;
activating the determined production program packet on the determined processing unit in order to decrypt the determined production program packet and then to control the processing unit to carry out one or more treatment steps on the educt based on the decrypted production program packet; and
determining a production program packet of the production program which follows the decrypted production program packet and is in particular encrypted and a following processing unit in order to execute this following production program packet, based on the decrypted production program packet.

It is to be understood that all structural and/or functional features which are linked with the system according to the invention described above and/or below, i.e. the production system according to the invention, and its embodiments, can also be comprised in said method either alone or in combination and the properties, designs, and advantages connected thereto can accordingly likewise be comprised and achieved.

The context of the above-described integrity of a processing unit will be described in more detail hereinafter.

In particular, the production system can comprise a or the above-described at least one control unit, which can be designed and programmed to determine a or the integrity of each processing unit, in particular before an educt can be accommodated and/or treated by means of a processing unit according to one of multiple production programs for manufacturing one of multiple products that can be manufactured by means of the production system.

A goal of the integrity determination can in particular be being able to establish changes to the processing units, in particular the patient boxes and/or the tool boxes. For example, it can therefore be detectable whether means of spying such as cameras and/or other comparable hardware have been attached by unauthorized parties in or on the processing units in order to be able to draw inferences about the manufacture of the product.

It can be provided that the computing unit of a processing unit is designed and programmed to provide a unique item of information related to this processing unit, and a further unique item of information related to this processing unit is detectable by means of the at least one control unit, wherein the integrity of this processing unit is determinable based on a comparison of these two unique items of information in particular by means of the at least one control unit and in particular is settable and/or confirmed upon correspondence of these two unique items of information.

Setting and/or confirming of the integrity can take place in particular in a respective computing unit and/or the at least one control unit.

In particular, the unique information that can be provided on the part of the computing unit can represent a setpoint status and/or a starting status of the processing unit, on the basis of which a deviation with respect to the processing unit is determinable.

A processing unit can comprise a security module, in particular a trusted platform module, for providing a unique item of information, in particular an item of fingerprint information about the processing unit, wherein the computing unit of the processing unit is designed and programmed to provide the unique item of information of the at least one control unit to determine the integrity.

In particular, the unique information or an item of fingerprint information can be understood as an item of digital fingerprint information, for example an item of information which is unique and/or comprises a unique identifier.

The production system can comprise one or more sensor units, by means of which the processing units are in particular visually detectable, and on the basis of this detection a unique item of information with respect to the respective processing units can be provided by means of the at least one control unit to determine the integrity.

In particular, the one or more sensor units can comprise one or more camera units, by means of which characteristic landmarks of a face, in particular a surface, and/or a geometry of the processing units are visually detectable.

In other words, the unique information can be based on characteristic landmarks of a face, in particular a surface, and/or a geometry of the processing units.

The geometry can be a two-dimensional and/or a three-dimensional geometry.

Additionally or alternatively, the production system can comprise one or more actuator units, by means of which one or more processing units can be mechanically excited in particular to generate an in particular mechanical oscillation, which is detectable by means of the one or more sensor units and on the basis of which a unique item of information with respect to the respective processing units can be provided by means of the at least one control unit to determine the integrity.

In other words, the unique information can be based on a characteristic response of the relevant processing unit with respect to the mechanical excitation. In case of a change, which is unauthorized in particular, on the relevant processing unit, this can be inferable from a deviation of the characteristic response with respect to a stored characteristic response.

It can be provided that at least one of the one or multiple actuator units is configured to generate a mechanical oscillation and this actuator unit can be arranged on or in one of the one or more processing units and/or can be able to be engaged with one of the one or more processing units to generate the mechanical oscillation at the relevant processing unit.

For example, an actuator unit can be designed as a motor, in particular a vibration motor, by means of which predeterminable frequency ranges can be generated and/or driven.

The oscillations can be detectable by means of at least one of the sensor units and a course of the oscillations specific for a processing unit can now be able to be provided as a characteristic response and/or as unique information, by which in particular structural changes in or on the processing unit are detectable.

In particular, the oscillation can comprise a frequency range of greater than or equal to 10 Hz and less than or equal to 100 Hz, and/or the oscillation can comprise one or more in particular different frequency ranges, which can respectively be greater than or equal to 10 Hz and less than or equal to 100 Hz, and/or the oscillation can comprise one or more in particular different frequency ranges which can follow one another in a specific order.

Additionally or alternatively, at least one of the one or multiple actuator units can be configured to generate an acoustic oscillation, in particular a soundwave, and this actuator unit can be arranged on or in one of the one or more processing units and/or can be able to be engaged with one of the one or more processing units to generate the mechanical oscillation, in particular the soundwave, with respect to the relevant processing unit.

For example, an actuator unit can be designed as a sound transducer, in particular a loudspeaker, by means of which predeterminable frequency ranges can be generated and/or driven.

The sound or the sound frequencies can be detectable by means of at least one of the sensor units and a course of the sound or the sound frequencies specific for a processing unit can now be able to be provided as a characteristic response and/or as unique information, by which in particular structural changes in or on the processing unit are detectable.

In particular, the (acoustic) oscillation can comprise a frequency range of greater than or equal to 30 Hz and less than or equal to 10 000 Hz, and/or the (acoustic) oscillation can comprise one or more in particular different frequency ranges, which can respectively be greater than or equal to 30 Hz and less than or equal to 10 000 Hz, and/or the (acoustic) oscillation can comprise one or more in particular different frequency ranges which can follow one another in a specific order.

Additionally or alternatively, it can be provided that one or more, in particular all processing units comprise at least one receptacle chamber, in particular enclosed by a lateral surface, for accommodating one or more objects, in particular an educt and/or one or more objects for treating the educt, wherein the integrity comprises an internal integrity which relates to the receptacle chamber, and/or comprises an external integrity which relates to an exterior with respect to the receptacle chamber.

It is to be understood that the described sensor units and/or the described actuator units can be configured with respect to the internal integrity and/or the external integrity.

In particular, the patient boxes and/or the tool boxes can be such processing units, which comprise the receptacle chamber enclosed in particular by a lateral surface.

It can be provided that a processing unit is designed and programmed to execute instructions according to at least one production program, in particular a production program packet, in particular to decrypt and then execute them, if its integrity is set and/or confirmed.

In other words, it can be provided that one or more, in particular all of the production program packets are provided on one processing unit in each case and can only be executed, in particular can be decrypted and executed, if the integrity of the relevant processing unit is set and/or confirmed.

Additionally or alternatively, one or more, in particular all of the production program packets can be transmittable to one processing unit in each case, in particular by means of the at least one control unit and/or by means of a computer system which is associated or can be associated with the production system, if the integrity of the relevant processing unit is set and/or confirmed.

It can be provided that a processing unit is designed and programmed to block an execution and/or a decryption of instructions according to at least one production program if their integrity cannot be set and/or cannot be confirmed.

In particular, a processing unit can be designed and programmed to provide a message, in particular an alarm message, in particular to the at least one control unit and/or a computer system which is associated or can be associated with the production system, if their integrity cannot be set and/or cannot be confirmed.

It can additionally or alternatively be provided that the at least one computing unit is designed and programmed to determine an integrity of the respective processing unit, in particular before an educt can be accommodated and/or treated by means of this processing unit according to one of multiple production programs for manufacturing one of multiple products that can be manufactured by means of the production system.

In other words, each processing unit can be designed as a self-checking processing unit, which is configured to determine its own integrity or in particular to be able to determine unauthorized changes to it itself.

It can be provided in particular that the integrity can be determinable by means of the at least one control unit and/or by means of each computing unit of the relevant processing unit.

In a certain sense, in other words, the production system can comprise at least one or the above-described at least one control unit, which is designed and programmed to determine an integrity of each processing unit, and each computing unit of a processing unit can be designed and programmed to determine an integrity of the respective processing unit, wherein optionally the respective integrity result can be compared for the plausibility check, in particular by means of the at least one control unit and/or the respective computing unit.

It can be provided that the computing unit of a processing unit is designed and programmed to store and/or provide a unique item of information related to this processing unit, and to detect a further unique item of information related to this processing unit, wherein the integrity of this processing unit is determinable based on a comparison of these two unique items of information by means of the computing unit and in particular is settable and/or confirmed upon correspondence of these two unique items of information.

In particular, the unique item of information can comprise a starting status of the relevant processing unit and a starting timestamp that can be associated with the starting status and the further unique item of information can comprise a further status of the relevant processing unit and a further timestamp that can be associated with the further status and is different from the starting timestamp.

As already described, the starting status can correspond to a setpoint status of the processing unit.

A unique chronological association and/or classification of the respective statuses of the processing unit can be carried out by means of the respective timestamp.

In particular processing units configured as patient boxes and/or as tool boxes can comprise at least one receptacle chamber, in particular enclosed by a lateral surface, for accommodating one or more objects, in particular an educt and/or one or more objects for treating the educt, wherein in particular the integrity comprises an internal integrity which relates to the receptacle chamber, and/or comprises an external integrity which relates to an exterior with respect to the receptacle chamber.

A processing unit, in particular a patient box and/or a tool box, can comprise one or more sensor units, by means of which a or the unique item of information and/or a or the further unique item of information are detectable via the relevant processing unit to determine the integrity, in particular an internal integrity.

In particular, a brightness value in and/or on the relevant processing unit can be detectable by means of at least one sensor unit and the unique item of information and/or the further unique item of information can be able to be provided via the relevant processing unit based on the detectable brightness value to determine the integrity, in particular an internal integrity.

Additionally or alternatively, the brightness value can be detectable continuously, in particular over an entire treatment procedure, to determine an integrity of the respective processing unit, while an educt is accommodated and/or treated by means of this processing unit according to one of multiple production programs for manufacturing one of multiple products that can be manufactured by means of the production system.

The at least one receptacle chamber enclosed in particular by a lateral surface can be configured as a chamber closed in operation of the relevant processing unit, so that an integrity cannot be set and/or cannot be confirmed upon detection of light. In other words, there can be a manipulation and/or an attack on the relevant processing unit.

Additionally or alternatively, it can be provided that a processing unit comprises one or more actuator units, by means of which the relevant processing unit can be mechanically excited in particular to generate an in particular mechanical oscillation, which is detectable by means of one or more sensor units and on the basis of which the unique item of information and/or the further unique item of information with respect to the relevant processing unit can be provided to determine the integrity.

In other words, the actuator units used for the integrity determination can be comprised in the respective processing units themselves and/or can be comprised in the production system, in particular outside the respective processing units.

For example, an actuator unit can be designed as a motor, in particular a vibration motor, by means of which predeterminable frequency ranges can be generated and/or driven.

It can be provided that the oscillation comprises a frequency range of greater than or equal to 10 Hz and less than or equal to 100 Hz, and/or the oscillation comprises one or more in particular different frequency ranges, which are respectively greater than or equal to 10 Hz and less than or equal to 100 Hz, and/or the oscillation comprises one or more in particular different frequency ranges, which follow one another in a specific order, wherein in particular at least one of the one or more actuator units is designed as a vibration motor, which is fixed in and/or on the relevant processing unit to generate the mechanical oscillation.

Additionally or alternatively, it can be provided that at least one of the one or more actuator units is configured to generate an acoustic oscillation, in particular a sound wave, and is fixed in and/or on the relevant processing unit to generate the mechanical oscillation, in particular the soundwave, with respect to the relevant processing unit.

In particular, it can be provided that the oscillation comprises a frequency range of greater than or equal to 30 Hz and less than or equal to 10 000 Hz, and/or the oscillation comprises one or more in particular different frequency ranges, which are respectively greater than or equal to 30 Hz and less than or equal to 10 000 Hz, and/or the oscillation comprises one or more in particular different frequency ranges which follow one another in a specific order.

The actuator units described with respect to the processing units for determining the integrity can be essentially configured like and/or can be functionally equivalent to the actuator units described with respect to the production system for determining the integrity.

Additionally or alternatively, a processing unit can comprise one or more electrically conductive line elements, which are arranged in and/or on the processing unit and/or are arranged inside a lateral surface wall of the processing unit and which form an in particular independent electric circuit, wherein it is detectable by means of one or more sensor units whether the electric circuit is changed with respect to its original status and/or is interrupted, and wherein the item of unique information and/or the further item of unique information about the relevant processing unit can be provided to determine the integrity on the basis of the circuit detection.

In other words, a processing unit can be provided and/or covered with a wire mesh network, in particular with one or more isolated wires, in particular in an interior, an exterior, and/or in an intermediate layer in relation thereto, wherein an intactness of the wire mesh network or the wires can be checked by means of a current source, so that if the electric current is absent, a manipulation and/or an attack can be determined and/or established.

Additionally or alternatively, at least one sensor unit can be designed as an inertial measuring unit, by means of which at least one acceleration value, in particular an acceleration vector, and/or at least one rotation rate value, in particular a rotation rate vector, is detectable with respect to the relevant processing unit and the unique item of information and/or the further unique item of information about the relevant processing unit can be provided based on the detectable acceleration and/or rotation rate values to determine the integrity.

In particular, a processing unit can be movable or moved by means of an external excitation or force in a predefined pattern, for example, forward-back-rotate-up-down, and the acceleration and/or rotation rate values of the processing unit resulting therefrom can be detectable by means of the inertial measuring unit in order to provide the unique item of information and/or the further unique item of information for comparison, so that in case of a deviation of the unique items of information or in case of different resulting acceleration and/or rotation rate values, a manipulation and/or an attack can be determined and/or established.

In particular, the production system can comprise a or the above-described handling device for moving an object within the production system and/or one or more actuator units, by means of which the relevant processing unit can be mechanically excited to generate an in particular defined movement, which is detectable by means of the inertial measuring unit and on the basis of which the unique item of information and/or the further unique item of information about the relevant processing unit can be provided to determine the integrity.

The handling device can in particular be the above-described multiaxis robot arm or can comprise a or this robot arm.

Additionally or alternatively, said acceleration and/or rotation rate values can in particular be continuously detectable during operation of the relevant processing unit by means of the inertial measuring unit, so that in particular for processing units of the same type, in particular design and/or configuration, the unique information can in particular be provided continuously for in particular continuous integrity determination. For example, said acceleration and/or rotation rate values of a processing unit, in particular a patient box and/or a tool box, can be detectable during the transport to a or the above-described cleaning device and/or to storage spaces and in particular a change determinable therefrom with respect to a mass and/or a mass distribution can be determinable, so that in this way a manipulation and/or an attack can be determined and/or established.

It can additionally or alternatively be provided that a temperature value in and/or on the relevant processing unit is detectable by means of at least one sensor unit and the unique item of information and/or the further unique item of information about the relevant processing unit can be provided based on the detectable temperature value to determine the integrity.

It can additionally or alternatively be provided that the relevant processing unit is at least partially visually detectable by means of at least one sensor unit and the unique item of information and/or the further unique item of information about the relevant processing unit can be provided based on the visual detection to determine the integrity, wherein in particular the at least one sensor unit comprises a camera unit, by means of which characteristic landmarks and/or geometries of the relevant processing unit are visually detectable.

It is to be understood that any combination of the components described with respect to the integrity, in particular actuator units and/or sensor units, can be conceivable with respect to the respective processing units.

Therefore, in other words, by means of the detection of sensor data or items of information or values at the beginning of a use and/or operation in the production system, a unique item of information or a digital fingerprint of the relevant processing units can be able to be generated. At an arbitrary time, at which a relevant processing unit is not filled with contents, e.g., tools, educts, etc., it can be checkable whether an integrity exists or whether no manipulation and/or no attack exists, in other words whether the unique information is still unchanged.

In particular, individual sensor units, such as brightness sensors, can also be usable during the operation, in particular continuously, for the integrity determination in order to thus establish potential attacks.

As already described, the multiple processing units can be used to accommodate and/or to treat an educt, wherein each processing unit can comprise at least one computing unit and wherein an educt can be able to be accommodated and/or treated by means of a processing unit according to one of multiple production programs for manufacturing one of multiple products that can be manufactured by means of the production system.

It can be provided in this case in particular that at least a part of a or the production program is in particular used exclusively to emulate and/or suggest one or more production program procedures, in particular one or more production program procedures of a manufacture of a product.

In this way, it can be achievable in particular that real or actual production steps or production program procedures are or become concealed, so that in particular potential attackers would have to employ further examinations to be able to reconstruct a manufacture of a product.

A production program procedure can in particular relate to an actual execution of instructions of a production program packet.

In particular, the part of a or the production program can in particular comprise one or more processing procedures exclusively for emulating and/or suggesting one or more production program procedures, which are additional and/or useless with respect to an actual manufacture of a product.

As already described, it can be provided that a production program comprises multiple, in particular encrypted production program packets, wherein each production program packet contains at least one item of information which refers to another production program packet and/or comprises instructions for accommodating and/or treating an educt, wherein in particular a defined order of the multiple production program packets forms the production program for manufacturing a product that can be manufactured by means of the production system.

In this case, it can be provided in particular that a production program comprises one or more, in particular encrypted dummy production program packets, wherein each dummy production program packet contains at least one item of information which refers to a production program packet or another dummy production program packet and/or which comprises dummy instructions for emulating and/or suggesting an accommodation and/or a treatment of an educt.

For example, a dummy production program packet, in particular a dummy instruction, can in particular be empty of content for production technology.

It can be provided that the production system comprises at least one random module, which is executable by means of one or more computing units of the processing units, by means of one or the above-described at least one control unit of the production system, and/or by means of a computer system that is associated or can be associated with the production system and by means of which one or more dummy production program packets can be integrated in a production program in a random manner, wherein in particular one or more dummy instructions can be associated with each dummy production program packet in a random manner by means of the at least one random module.

A processing unit is designed and programmed to execute, in particular to decrypt and then execute, any instructions, in particular actual instructions for accommodating and/or treating an educt and/or dummy instructions, according to a production program.

In particular, a processing unit can be designed and programmed to accommodate and/or treat a dummy educt, in order to emulate and/or suggest an accommodation and/or a treatment of an actual educt using the dummy educt.

For example, a dummy educt can be an empty container intended for an actual educt, which cannot be seen into from the outside, and/or can comprise a material similar to an actual educt.

Additionally or alternatively, it can be provided that the production system comprises one or more concealment chambers for accommodating one or more processing units, wherein an interior of each concealment chamber is optically separated and/or at least largely cannot be seen into from outside the concealment chamber, wherein in particular at least one processing unit can be prompted by means of the at least one part of a production program for emulating and/or suggesting a manufacture of a product in order to be accommodated in the interior of a relevant concealment chamber.

For example, it can be substantially or completely not possible to see into a concealment chamber for persons who stand outside the production system, in particular the clean room area.

For example, an optical separation can relate to a wavelength range visually perceptible by humans and/or to a wavelength range not visually perceptible by humans, for example an infrared range.

It can be provided that a storage space of the storage device is configured as a concealment chamber. Additionally or alternatively, a concealment chamber can be arranged as an independent apparatus in the clean room area.

It can be provided that at least one concealment chamber comprises one or more changing units, by means of which objects which are accommodated in and/or by processing units, in particular patient boxes and/or tool boxes, can be rearranged between the relevant processing units which are located inside the concealment chamber.

Additionally or alternatively, it can be provided that at least one concealment chamber comprises one or more storage spaces on which one or more processing units can be accommodated for a random period of time.

At least one concealment chamber can exclusively be configured to emulate and/or suggest a treatment of an educt. Alternatively, at least one concealment chamber can be configured for the (actual) treatment of an educt and/or for the emulation and/or suggestion of a treatment of an educt.

It can be provided that at least one concealment chamber comprises one or more sensor units for monitoring the optical separation of the interior from the exterior of the concealment chamber, wherein in particular a brightness value inside the concealment chamber is detectable by means of at least one sensor unit of the concealment chamber.

It can be provided that at least one dummy production program packet comprises one or more dummy instructions which cause an accommodation of one or more processing units in one of the concealment chambers.

In other words, a processing unit can be able to be instructed by means of at least one dummy production program packet in order to be able to be accommodated or to be accommodated in one of the concealment chambers.

It can be provided that at least one dummy production program packet comprises one or more dummy instructions which cause a rearrangement of objects, which are accommodated in and/or by processing units, in particular patient boxes and/or tool boxes, between the relevant processing units, which are located inside the concealment chamber.

It can additionally or alternatively be provided that the production system comprises a or the above-described at least one control unit, which is designed and programmed to at least partially store and operate a database and/or to exchange items of information with a database, which is at least partially stored and/or operated on a computer system that is associated or can be associated with the production system, wherein the database contains one or multiple, in particular unique items of release information for authorizing an accommodation and/or treatment of an educt by means of one of the multiple processing units, wherein the database is changeable by a retrieval of at least one of the items of release information for one of the multiple processing units so that this release information subsequently can no longer be provided in the original form for retrieval.

In particular, the unique release information can be understood as an item of information which is unique and/or comprises a unique identifier.

It can be provided that the production system comprises the computer system which is associated or can be associated with the production system.

Additionally or alternatively, it can be provided that the computer system which is associated or can be associated with the production system is a release computer system, in particular a cloud-based release computer system and/or a release server.

The database can be configured and programmed to receive and store one or more new items of release information exclusively from outside the production system. For example, one or more new items of release information can be transmittable to the database on the part of a database access authorized party, in particular a rights holder of a formula represented by a production program, in particular via a computer system, for example via a cloud and/or a server.

Additionally or alternatively, the database can be configured and programmed to be storable and/or operable exclusively on the computer system that is associated or can be associated with the production system, or to be able to be synchronized with a parallel database, which is storable and/or operable exclusively on the computer system that is associated or can be associated with the production system.

It can be provided that each computing unit of each processing unit is configured and programmed to receive and store one or more items of release information, wherein each item of release information received by a computing unit can be compared to each item of release information already stored on a computing unit to avoid a double use of the received release information in the production system.

In particular, each computing unit can be designed and programmed to at least partially or completely store and operate an in particular encrypted decentralized database, preferably an in particular encrypted distributed ledger, in order to store the one or more receivable or received items of release information. In particular a manipulation-secured technical solution can be provided by the use of the decentralized database in order to avoid a double use of the received release information in the production system. The decentralized database is described in more detail in the following description.

As was already described, one of multiple products can be able to be manufactured according to one of multiple production programs by means of the production system, wherein a production program can comprise multiple production program packets and each production program packet can contain at least one item of information which comprises instructions for accommodating and/or treating an educt.

It can be provided in this case that at least one of the items of release information can be uniquely associated with each production program packet to authorize an in particular one-time execution of this production program packet by means of at least one processing unit. It can be ensured in this way that the execution of production program packets is not possible beyond a released limit or a released execution number.

In particular, each production program can only be able to be initialized and/or started by means of the production system if all production program packets of the production program and/or all items of release information associated with the production program packets can be provided or are provided.

It can be provided that one or more items of release information are receivable and storable by means of at least one processing unit, in particular its computing unit, for a time-delayed authorization and execution of an accommodation and/or a treatment of an educt and/or one or more items of release information are receivable by means of at least one processing unit, in particular its computing unit, in real time for an immediate authorization and execution of an accommodation and/or a treatment of an educt.

Said authorization and execution of an accommodation and/or treatment of an educt can in particular relate to an authorization of an execution of instructions for accommodating and/or treating an educt of a production program packet and to the corresponding actual execution.

The database can be able to be encrypted or can be encrypted and/or each item of release information in the database can be able to be encrypted or can be encrypted.

For example, at least a part of the database and/or at least one item of release information can be able to be decrypted by means of at least one cryptographic key which can be provided and/or stored by means of a processing unit and/or is stored there.

A cryptographic key with respect to a release can be different from a cryptographic key with respect to a production program packet.

Additionally or alternatively, such cryptographic keys can in particular be private cryptographic keys, in particular for a public-key encryption.

As already described, one of multiple products can be able to be manufactured according to one of multiple production programs by means of the production system, wherein at least a part of a production program can be used in particular to emulate and/or suggest a manufacture of a product. It can be provided in this case that this at least one part of a production program can be authorized for execution by means of one or more items of release information.

In other words, each production program can only be able to be initialized and/or started by means of the production system if all production program packets including the dummy production program packets of the production program and/or all items of release information associated with the production program packets including the dummy production program packets can be provided or are provided.

One or more items of release information can be able to be retrieved from the database as bundled release information for authorizing an overall process for manufacturing a product, in particular by means of one or more processing units, and/or can be provided by means of the database. In this way, a release process can be able to be simplified, since each individual item of release information does not have to be separately released on the part of a database access authorized party, in particular a rights holder for a formula represented by a production program, but rather in particular entire production programs can also be releasable at once.

Additionally or alternatively, it can be provided that an item of release information is retrievable individually from the database for each individual production program packet, in particular by means of a processing unit, and/or can be provided by means of the database, and/or multiple items of release information for production program packets which can be associated are retrievable together from the database, in particular by means of one or more processing units, and/or can be provided by means of the database.

The database can be configured and programmed to associate one or more, in particular all items of release information with a respective item of confirmation information, which is storable in the database for each item of release information, in particular by a database access authorized party, wherein each item of release information can only be provided if an associated item of confirmation information is stored.

Additionally or alternatively, the database can be configured and programmed to contain and provide various types of release information, wherein in particular a type of a release corresponds with a type of a processing unit and/or with a type of a part, in particular a production program packet, of a production program for manufacturing a product.

For example, a type of release information can relate only to patient boxes and/or only to tool boxes, or a type of release information can relate to a type of treatment procedure, for example a temperature control and/or a storage procedure in the storage device.

It can be provided that each item of release information comprises an information stamp, in particular a mode information stamp, by means of which the respective item of release information can be associated with a production mode of the production system for manufacturing a product and/or a simulation mode of the production system for simulating the operation of the production system and/or in particular the manufacturing of a product.

In other words, depending on the information stamp of the release information, a production program packet can be executable by means of a processing unit for actually accommodating and/or treating an educt or only for its simulation with respect to the relevant processing unit.

Additionally or alternatively, each item of release information can comprise an information stamp, in particular a number information stamp, by means of which an in particular maximum repeatability of an accommodation and/or a treatment of an educt is settable, which can be authorized by means of the relevant release information. It is to be understood that this one in particular maximum repeatability can comprise a simulation of the operation of the production system and/or in particular the manufacturing of a product.

It can be provided that the at least one control unit is designed and programmed to be connectable or connected for data exchange to the computer system that is associated or can be associated with the production system to determine an existence of this data connection, wherein the production system and/or the one or more, in particular all processing units are reversibly transferable depending on the data connection into a normal status for unrestricted operation, into a reserve status for chronologically and/or functionally restricted operation, and/or into a pausing status for idled operation. For example, this computer system can be configured as a heartbeat computer system, which will be described in more detail in the following description. It can be provided in particular that the production system comprises this computer system in particular this heartbeat computer system, which is associated or can be associated with the production system. Additionally or alternatively, it can be provided that this computer system which is associated or can be associated with the production system is a cloud-based computer system and/or a server.

It can be provided that each computing unit is designed and programmed to at least partially or completely store and operate an in particular encrypted decentralized database, preferably an in particular encrypted distributed ledger, wherein each decentralized database is a redundant copy of each of the other decentralized databases, wherein each computing unit is designed and programmed to validate an operation of the respective decentralized database on each of the other computing units and to update its own decentralized database in response to a validation, wherein each decentralized database is designed and programmed to comprise one or more, in particular unique items of information about an accommodation and/or a treatment of an educt by each processing unit.

In particular, the unique information can be understood as an item of information which is unique and/or comprises a unique identifier.

As already described, each processing unit, in particular the computing unit of each processing unit, can comprise a or the security module, in particular a trusted platform module, for providing one or more cryptographic keys.

Such a cryptographic key can in particular be a private cryptographic key in particular for a public-key encryption.

Each decentralized database can be able to be encrypted or can be encrypted by means of a cryptographic key, which can only be associated with and/or provided to the processing unit on which the decentralized database is storable or stored. It can be ensured in this way that the decentralized database is only storable a single time on the processing unit.

As was already described, one of multiple products can be able to be manufactured according to one of multiple production programs by means of the production system, wherein a production program can comprise multiple production program packets and each production program packet can contain at least one item of information which comprises instructions for accommodating and/or treating an educt, which are executable by means of at least one processing unit. It can be provided in this case that each computing unit is configured and programmed to store each execution of a production program packet in the decentralized database, in particular depending on the validation by the other computing units.

It can be provided that each computing unit is configured and programmed to execute a production program packet only if this production program packet is not stored as already executed in the decentralized database.

In particular, each computing unit can be configured and programmed to receive one or more, in particular unique items of release information for authorizing an accommodation and/or treatment of an educt from a or the above-described at least one control unit of the production system and/or a computer system that is associated or can be associated with the production system, in particular a or the release computer system, and to store the one or more items of release information in its own decentralized database.

Optionally, each computing unit can be configured and programmed to compare each received item of release information with each item of release information already stored in the decentralized database to avoid a double use of the received release information in the production system, in particular depending on the validation by the other computing units.

It can be provided that each item of release information comprises an information stamp, in particular a number information stamp, by means of which an in particular maximum repeatability of an accommodation and/or treatment of an educt is settable, which can be authorized by means of the relevant release information, wherein in particular the information stamp, in particular the number information stamp, of each item of release information can be updated in the decentralized database depending on an authorized execution of an accommodation and/or a treatment of an educt.

Each production program packet can be able to be uniquely associated with at least one of the items of release information to authorize an execution of this production program packet by means of at least one processing unit, as was already described above.

In particular, each computing unit can be designed and programmed to execute the instructions of a production program packet only if an item of release information associated with this production program packet is provided to the computing unit and in particular this production program packet and/or this release information is stored in the decentralized database as not yet already executed.

Additionally or alternatively, each computing unit can be designed and programmed to execute the instructions of a production program packet only if a maximum repeatability of an execution of this production program packet is stored as not yet reached according to the release information in the decentralized database.

In other words, a permission to execute a production program packet can be able to be granted by means of the described release information mechanism, which permission is storable in particular in the decentralized database, wherein it is checkable in particular upon each execution of a production program packet whether the number of the previous executions is less than the permitted number of executions of the production program packet, and wherein it is storable in particular in the decentralized database that a production program packet will be or is executed or can be executed, by which said number of available executions is reducible by 1 (one) upon actual execution. It can be ensured in this way that the execution of production program packets is not possible beyond the released limit.

It can be provided that the production system comprises one or more further computer systems and/or is connectable or connected to such further computer systems, wherein each of the further computer systems is arranged in a different location with respect to a remainder of the production system, wherein the decentralized database is stored and operated on each of the further computer systems, and wherein each of the further computer systems is designed and programmed to validate an operation of the respective decentralized databases on each of the other computer systems and/or computing units and to update its own decentralized database in response to a validation.

For example, these further computer systems can in particular be cloud-based computer systems and/or servers.

As already described, the production system can comprise a or the above-described at least one control unit, which is designed and programmed to be connectable or connected for data exchange to a computer system that is associated or can be associated with the production system, in particular the above-described heartbeat computer system, to determine an existence of this data connection.

This computer system, in particular the heartbeat computer system, is to be described in more detail hereinafter.

In particular, this computer system can be used so that the production system can only be operated over a specific time without data connection. In other words, additionally or alternatively to the requirement for the above-described items of release information, the production system can only produce if a connection exists to said computer system, in particular the heartbeat computer system.

It can be provided that the production system and/or the one or more, in particular all processing units are reversibly transferable depending on the data connection into a normal status for unrestricted operation, into a reserve status for chronologically and/or functionally restricted operation, and/or into a pausing status for idled operation.

The reserve status can in particular be a transition status between the normal status and the pausing status, via which the normal status passes into the pausing status, wherein in particular a transition from the pausing status to the normal status does not have to take place via the reserve status and/or preferably a transition from the pausing status to the normal status can take place directly.

In particular, the at least one control unit and/or one or more, in particular each of the computing units can be designed and programmed to determine the existence of the data connection to the computer system, in particular continuously or at regular time intervals.

It can be provided that the production system is only unrestrictedly operable to manufacture a product, in particular the processing units to accommodate and/or treat an educt, upon the existence of the data connection. This can be the normal status and/or can be associated with the normal status.

Additionally or alternatively, it can be provided that if the data connection does not exist, the production system for manufacturing a product, in particular the processing units for accommodating and/or treating an educt, is transferable into a reserve status, in which the manufacturing of a product, in particular the accommodation and/or the treatment of an educt, is chronologically and/or functionally restricted.

The at least one control unit and/or one or more, in particular each of the computing units can be designed and programmed to store an information stamp, in particular a timestamp, upon transfer into the reserve status and to stop an unrestricted operation of the production system and/or the one or more, in particular all processing units after a predefined period of time from this information stamp, in particular this timestamp, and in particular to transfer the production system and/or the relevant processing units into the pausing status.

In particular, the at least one control unit and/or one or more, in particular each of the computing units can be designed and programmed, upon transfer into the reserve status, to only still execute a predefined part of a production program for manufacturing a product. This can be used, for example, to ensure production or treatment progress on an educt and/or to bridge a possible short-term interruption of the data connection. In particular if the reserve status has passed or gone back into a normal status and said predefined part of the production program was not yet completely executed, the production program can be continued normally.

Additionally or alternatively, the at least one control unit and/or one or more, in particular each of the computing units can be designed and programmed, upon passing into the pausing status, to modify and/or supplement a production program for manufacturing a product using one or more production program packets to store an educt and/or an at least partially treated educt and/or a product, in particular as long as the pausing status exists.

Storing can take place in particular in at least one of the storage spaces of the storage device.

A modification and/or a supplementation can comprise, for example, targeted temperature control, for example freezing, of an educt and/or an at least partially treated educt and/or product in order to secure a production or treatment progress.

The computer system, in particular the heartbeat computer system, can be arranged in a different location with respect to a remainder of the production system, wherein in particular the computer system comprises a server to which the at least one control unit is connectable or connected for data exchange.

It can be provided that the production system is configured to be operable in particular using the following steps, and/or in other words, a method can be provided for in particular partially controlling a or the production system to manufacture a product, in particular a biological-pharmaceutical product, wherein the production system is configured as described above and/or below and wherein the method comprises the following:
providing one or more processing units for accommodating and/or treating an educt;
providing a data connection from a computer system which is associated or can be associated with the production system to the production system and/or one or more, in particular all processing units;
determining whether the data connection exists by means of at least one control unit of the production system and/or at least one computing unit of at least one processing unit; and
controlling the production system and/or the one or more, in particular all processing units depending on the data connection.

It can be provided that the control comprises a reversible transfer of the production system and/or the one or more, in particular all processing units into a normal status for unrestricted operation, into a reserve status for chronologically and/or functionally restricted operation, and/or into a pausing status for idled operation, in particular based on the existence of the data connection.

It can be provided that the at least one control unit and/or one or more, in particular each of the computing units store an information stamp, in particular a timestamp, upon transfer into the reserve status and stop an unrestricted operation of the production system and/or the one or more, in particular all processing units after a predefined period of time from this information stamp, in particular this timestamp, and in particular transfer the production system and/or the relevant processing units into the pausing status.

It can be provided that the at least one control unit and/or one or more, in particular each of the computing units, upon transfer into the reserve status, only still execute a predefined part of a production program for manufacturing a product.

It can be provided that the at least one control unit and/or one or more, in particular each of the computing units, upon passing into the pausing status, modifies and/or supplements a production program for manufacturing a product using one or more production program packets to store an educt and/or an at least partially treated educt and/or a product, in particular as long as the pausing status exists.

It is to be understood that all structural and/or functional features which are linked with the system according to the invention described above and/or below, i.e. the production system according to the invention, and its embodiments, can also be comprised in said method, in particular described above, either alone or in combination, and the properties, designs, and advantages connected thereto can accordingly likewise be comprised and achieved.

It can be provided that the production system comprises a simulation device, which is designed and programmed to simulate the operation of the production system and/or in particular the manufacture of a product based on at least one production program and to provide a simulation result corresponding thereto, wherein the simulation device is partially or completely formed by one or more, in particular all computing units of the respective processing units.

In particular, since the properties of the production system, i.e., for example, system-specific and/or tool-specific characteristics, can play a large role with respect to phasing in the production system and/or its capacity, said simulation of the production system can comprise a software-based and/or a hardware-based simulation. In other words, the simulation may not be able to be carried out solely in a software-based manner, for example on the basis of a digital production system twin, but rather can be carried out under the production system itself. This can comprise in particular that each component of the production system can be designed and programmed to participate in the simulation and/or carry out a part thereof.

For example, in order to avoid formulas or production program sequences or other information being disclosed to an unauthorized party by simulation, a distinction can be made between the simulation mode and the production mode or a simulation and an actual execution of a production program only with respect to carrying out actual physical movements, which then preferably do not take place in the simulation. The above-described procedures with respect to release, encryption/decryption, secured communication (see in particular description below), etc. can take place in the simulation.

It can be provided, for example, that the simulation device is designed and programmed to simulate a part of the associated production program which has not yet been executed during a manufacture of a product and to provide a simulation result corresponding thereto. This can be understood in particular to mean that at least a part of the production system, in particular one or more processing units of the production system, are dynamically switchable back-and-forth between a or the production mode and a or the simulation mode, in particular depending on the simulation requirement. In other words, starting from a current status, in particular production status, of the production system it is possible to further simulate based on the at least one production program, for example to optimize following production steps and/or treatment procedures and/or to determine which further and/or additional treatment procedures and/or production programs can be initiated.

The simulation device can be designed and programmed to detect system parameters of the production system during a manufacture of a product and/or to detect accommodation procedure measured values and/or treatment procedure measured values of the processing units, in particular operating parameters of the processing units, and store them as simulation information and save them to carry out a simulation, wherein in particular the simulation of an operation of the production system and/or a manufacture of a product can be carried out on the basis of the simulation information by means of the simulation device.

In other words, the simulation device can be designed and programmed to detect:
process-specific parameters and/or measured values, for example an order of treatment procedures or processing steps, and/or
system-specific parameters and/or measured values, for example of processing units and/or location parameters of the production system such as exterior temperature, ambient humidity, air pressure, and/or
patient-specific parameters and/or measured values, for example a medical treatment history, and/or
tool-specific parameters and/or measured values, for example of tool units, in particular tool boxes, and/or
material-specific parameters and/or measured values, for example of consumable materials, and to store them as simulation information and save them to carry out a simulation.

As described above, the production system can comprise one or more sensor units. It can be provided in this case that they are at least partially associated with the processing units and/or form a part of the processing units, wherein the simulation device is designed and programmed to carry out the simulation on the basis of items of simulation information detectable or detected by means of these sensor units.

In particular, the sensor units can be designed and/or configured as described in the application DE 10 2021 207 738.2, to which reference is hereby made.

In particular, an item of simulation information can comprise at least one of the following: time parameters and/or time measured values of the production system, in particular the processing units, climate parameters and/or climate measured values in and/or around the production system, in particular in and/or around the processing units, and/or operating parameters and/or its operating measured values of the production system, in particular the processing units.

For example, time parameters and/or time measured values can comprise: pumping periods of processing units configured as pumping devices and/or transport periods of processing units and/or sterilization times of processing units configured as sterilization devices and/or cleaning times of processing units configured as cleaning devices and/or waiting times within the production system and/or unpacking/packing times of processing units configured as unpacking/packing devices and/or loading/unloading times of processing units and/or fitting times of processing units configured as tool units.

Additionally or alternatively, time parameters and/or time measured values can comprise treatment time values of treatment procedures that can be carried out by means of processing units.

In particular, multiple, in particular all treatment procedures can comprise said treatment time values, which specify the period of time which passes from a start of the respective treatment procedure to its end, in particular including:
a period of time for providing the at least one tool and/or tool box required for the respective treatment procedure, in particular including equipping it with one or more tool consumable means, and/or
a period of time for feeding and/or arranging and/or putting into operation the at least one tool and/or tool box at the educt to be treated, in particular in a patient box in which the educt is arranged, and/or
a period of time of the action on the educt to achieve a desired treatment result, and/or a period of time for removing the at least one tool and/or tool box from the educt, in particular from the patient box;
preferably including:
   cleaning the participating processing units, in particular patient boxes and/or tool boxes, and/or
   removing and/or disposing of tool consumable means, and/or
   transporting the participating processing units, in particular patient boxes and/or tool boxes back to a starting location, in particular a respectively associated storage space.

For example, climate parameters and/or climate measured values can comprise: temperature values and/or ambient humidity values and/or gas composition values.

For example, operating parameters and/or operating measured values can comprise: temperature parameters of processing units configured as temperature control devices and/or fill levels of storage devices.

In particular, the part of a production program can be able to be simulated by means of the computing units forming the simulation device which is to be executed by means of the associated processing units for manufacturing a product, wherein in particular a production program comprises multiple, in particular encrypted production program packets and each production program packet contains at least one item of information which comprises instructions for accommodating and/or treating an educt, which are executable by means of at least one processing unit, and one or more production program packets form said part of the production program to be simulated.

In other words, each processing unit can be designed and programmed to simulate at least the part of a production program which the relevant processing unit has to and/or would execute to actually manufacture a product.

As already described, a production program can comprise multiple, in particular encrypted production program packets and each production program packet can contain at least one item of information, which comprises instructions for accommodating and/or treating an educt, which are executable by means of at least one processing unit, wherein it can be provided here that the simulation is executable based on one or more production program packets, wherein in particular the one or more production program packets are designed and programmed both for a manufacture of a product and also for simulating the operation of the production system and/or the manufacture of the product.

In other words, each processing unit can be designed and programmed to simulate at least one production program packet which the relevant processing unit has to and/or would execute to accommodate and/or treat an educt during an actual manufacture of a product.

A processing unit can be designed and programmed to store one or more cryptographic keys, by means of which production program packets of the production program can each be decrypted for the simulation.

Such a cryptographic key can in particular be a private cryptographic key in particular for a public-key encryption.

In particular, a cryptographic key for the simulation can be the same cryptographic key which is uniquely associated with a production program packet. Alternatively, the keys can be different and can be able to be respectively associated with the simulation and the actual production.

Each production program packet can be releasable for execution by means of an item of release information receivable by the processing unit for the simulation of the operation of the production system and/or a manufacture of a product.

In particular, each item of release information can comprise an or the information stamp, in particular a or the mode information stamp, by means of which the respective item of release information and/or the respective production program packet can be associated with a production mode of the production system for manufacturing a product and/or a simulation mode of the production system for simulating the operation of the production system and/or the manufacturing of a product.

Each processing unit can be designed and programmed to be operable in a production mode of the production system to manufacture a product and/or a simulation mode of the production system to simulate the operation of the production system and/or the manufacturing of a product.

A differentiation between the production mode and the simulation mode can in particular be able to be performed on the basis of the information stamp by the processing unit in order to be in a corresponding operation.

One or more processing units can be designed and programmed to simultaneously simulate one or more production program packets for at least one processing unit in the production mode and in particular to generate items of simulation information in this case.

For example, during an actual manufacture of a product by means of one or more processing units, any parameters and/or measured values detectable by means of the one or more sensor units can be storable as items of simulation information, in particular by means of the simulation device, for example the relevant computing units of the processing units participating in the actual manufacture of a product.

As already described, each production program packet can contain at least one item of information which refers to another production program packet and/or comprises instructions for accommodating and/or treating an educt, wherein in particular a defined order of the multiple production program packets can form the production program for manufacturing a product that can be manufactured by means of the production system. It can be provided that each reference to another production program packet comprises an or the information stamp, in particular a or the mode information stamp, by means of which the respective other production program packet can be associated with a production mode of the production facility for manufacturing a product and/or a simulation mode of the production facility for simulating the operation of the production facility and/or the manufacturing of a product.

The production system can comprise a or the above-described at least one control unit, by means of which an in particular logistical utilization and/or occupancy of the production system can be provided based on the simulation result.

Additionally or alternatively, the production system can comprise a or the above-described at least one control unit, by means of which, based on the simulation result, a prediction of a chronological production sequence of one or multiple production programs can be prepared and/or, based on the simulation result, the processing units required and/or intended for carrying out the treatment procedures and/or consumable materials to be provided in the production system can be associated chronologically and/or positionally.

In particular, an in particular statistically optimum entry point or start for a manufacture of a new or further product can be determinable by the simulation of the treatment procedures via various runtimes. Due to the simulation of an actual production which is in particular similar to the hardware, this production can take place reliably and very accurately.

As already described above, a simulation can also at least partially or completely require release by means of one or more items of release information. A release of a simulation can essentially take place analogously to a release of an actual manufacture of a product. This will be described in more detail hereinafter in reference to a simulation.

As described above, the production system can comprise the above-described simulation device. It can be provided in this case that the production system furthermore comprises a or the above-described at least one control unit, which is designed and programmed to at least partially store and operate a or the above-described database and/or to exchange items of information with a or the above-described database, which is at least partially stored and/or operated on a computer system that is associated or can be associated with the production system, wherein the database can in particular additionally and/or alternatively contain multiple, in particular unique items of release information for authorizing a simulation of an accommodation and/or treatment of an educt by means of one of the multiple processing units, wherein the database is changeable by a retrieval of at least one of the items of release information for a simulation by means of one of the multiple processing units so that this release information subsequently can no longer be provided in the original form for retrieval.

In particular, the unique release information can be understood as an item of information which is unique and/or comprises a unique identifier.

It can be provided that the production system comprises the computer system which is associated or can be associated with the production system.

Additionally or alternatively, it can be provided that the computer system which is associated or can be associated with the production system is a or the release computer system, in particular a or the cloud-based release computer system and/or a or the release server.

It can be provided that the database is configured and programmed to receive and store one or more new items of release information for authorizing a simulation exclusively from outside the production system. For example, one or more new items of release information can be transmittable to the database on the part of a database access authorized party, in particular a rights holder of a formula represented by a production program, in particular via a computer system, for example via a cloud and/or a server.

It can be provided that the database is configured and programmed to be storable and/or operable exclusively on the computer system that is associated or can be associated with the production system, or to be able to be synchronized with a parallel database, which is storable and/or operable exclusively on the computer system that is associated or can be associated with the production system.

It can be provided that each computing unit of each processing unit is configured and programmed to receive and store one or more items of release information, wherein each item of release information received by a computing unit can be compared to each item of release information already stored on a computing unit to avoid a double use of the received release information in the production system.

In particular, each computing unit can be designed and programmed to at least partially or completely store and operate an in particular encrypted decentralized database, preferably an in particular encrypted distributed ledger, in order to store the one or more receivable or received items of release information. In particular a manipulation-secured technical solution can be provided by the use of the decentralized database in order to avoid a double use of the received release information in the production system. The decentralized database is described in more detail in the following description.

As was already described, one of multiple products can be able to be manufactured according to one of multiple production programs by means of the production system, wherein a production program can comprise multiple production program packets and each production program packet can contain at least one item of information which comprises instructions for accommodating and/or treating an educt.

It can be provided in this case that at least one of the items of release information can be uniquely associated with each production program packet to authorize an in particular one-time simulation and/or an in particular one-time execution of this production program packet by means of at least one processing unit. It can be ensured in this way that the simulation of production program packets is not possible beyond a released limit or a released execution number.

In particular, a simulation can only be able to be initialized and/or started by means of the simulation device if all production program packets of the production program and/or all items of release information associated with the production program packets can be provided.

It can be provided that one or more items of release information are receivable and storable by means of at least one processing unit for a time-delayed authorization and execution of a simulation of an accommodation and/or a treatment of an educt and/or one or more items of release information are receivable by means of at least one processing unit in real time for an immediate authorization and execution of a simulation of an accommodation and/or a treatment of an educt.

It can be provided that the database is able to be encrypted or is encrypted and/or each item of release information in the database is able to be encrypted or is encrypted.

For example, at least a part of the database and/or at least one item of release information can be able to be decrypted by means of at least one cryptographic key which can be provided and/or stored by means of a processing unit and/or is stored there.

A cryptographic key with respect to a release can be different from a cryptographic key with respect to a production program packet.

Additionally or alternatively, such cryptographic keys can in particular be private cryptographic keys, in particular for a public-key encryption.

As already described, one of multiple products can be able to be manufactured according to one of multiple production programs by means of the production system, wherein at least a part of a production program can be used in particular to emulate and/or suggest a manufacture of a product. It can be provided in this case that this at least one part of a production program can be authorized for simulation and/or execution by means of one or more items of release information.

In other words, a simulation can only be able to be initialized and/or started by means of the simulation device if all production program packets including the dummy production program packets of the production program and/or all items of release information associated with the production program packets including the dummy production program packets can be provided or are provided.

It can be provided that one or more items of release information are retrievable from the database as bundled release information for authorizing a simulation of the operation of the entire production system and/or a simulation of an overall process for manufacturing a product, in particular can be provided by means of one or more processing units and/or by means of the database. In this way, a release process can be able to be simplified, since each individual item of release information does not have to be separately released on the part of a database access authorized party, in particular a rights holder for a formula represented by a production program, but rather in particular a complete simulation of an entire production program can also be releasable at once.

Additionally or alternatively, it can be provided that an item of release information is retrievable individually from the database for each individual production program packet, in particular by means of a processing unit, and/or can be provided by means of the database, and/or multiple items of release information for production program packets which can be associated are retrievable together from the database, in particular by means of one or more processing units, and/or can be provided by means of the database.

It can be provided that the database is configured and programmed to associate one or more, in particular all items of release information with a respective item of confirmation information, which is storable in the database for each item of release information, in particular by a database access authorized party, wherein each item of release information can only be provided if an associated item of confirmation information is stored.

Additionally or alternatively, it can be provided that the database is configured and programmed to contain and provide various types of release information, wherein in particular a type of a release corresponds with a type of a processing unit and/or with a type of a part, in particular a production program packet, of a production program for manufacturing a product.

For example, a type of release information can relate only to patient boxes and/or only to tool boxes, or a type of release information can relate to a type of treatment procedure, for example a temperature control and/or a storage procedure in the storage device.

As already described, it can be provided that each item of release information comprises an or the information stamp, in particular a or the mode information stamp, by means of which the respective item of release information can be associated with a production mode of the production system for manufacturing a product and/or a simulation mode of the production system for simulating the operation of the production system and/or in particular the manufacturing of a product.

Additionally or alternatively, it can be provided that each item of release information comprises an or the information stamp, in particular a or the number information stamp, by means of which an in particular maximum repeatability of a simulation of an accommodation and/or a treatment of an educt is settable, which can be authorized by means of the relevant release information.

Furthermore, it can be provided that the production system is switchable to another status depending on the existence of a data connection to the computer system which is associated or can be associated with the production system, in particular the heartbeat computer system, which was already described. It can be provided in this case that a simulation can be carried out, or not, depending on the respective status.

As described above, each processing unit can comprise one or more sensor units. It can be provided in this case that each sensor unit is configured to detect at least one measured value with respect to the accommodation and/or the treatment of the educt and/or with respect to the processing unit itself and to provide the measured value to the computing unit for transmission to one or more reception-authorized units in encrypted and/or abstracted form.

In this way, measured values and/or information can be made unidentifiable on the processing unit side, so that an information outflow, for example by means of a manipulative tap, to an unauthorized party is avoidable.

In other words, at least one measured value, which is detectable by means of the one or multiple sensor units, can first be made/become unidentifiable before it is transmittable to one or more reception-authorized units.

It can be provided that the one or more reception-authorized units comprise one or more other processing units and/or at least one higher-order control unit of the production system and/or a computer system which is associated or can be associated with the production system.

It can be provided that an item of information which represents the at least one detectable measured value is modifiable by the sensor unit itself and the one or more reception-authorized units are designed and programmed to interpret the modified information.

Additionally or alternatively, it can be provided that an item of information which represents the at least one detectable measured value is modifiable by means of the computing unit and the one or more reception-authorized units are designed and programmed to interpret the modified information.

Additionally or alternatively, it can be provided that the information is modifiable in an encrypted manner and/or in an abstracting manner and the information can be decrypted and/or made specific by means of the one or more reception-authorized units.

In other words, a modification can comprise an encryption and/or an abstraction.

As already described, an encryption can also refer here to a public-key encryption.

An abstraction can comprise, as a simple, nonrestrictive example, in particular an omission of in particular physical units. Only the one or more reception-authorized units can interpret the solely numeric value and/or assign the correct, in particular physical unit thereto again.

An information depth or an information content of the at least one measured value can therefore be able to be retained. In other words, therefore no information loss can occur.

It can be provided that each computing unit is configured and programmed to provide the information only or exclusively as modified information for transmission to the one or multiple reception-authorized units.

Additionally or alternatively, it can be provided that each computing unit is configured and programmed to assess the at least one detectable measured value according to parameter ranges storable or stored in the computing unit and subsequently to provide only a result of the assessment of the at least one detectable measured value for transmission to one or more reception-authorized units.

For example, the at least one detectable measured value can be assessable on a scale of 1 to 100 by means of the computing unit and only the determined scalar value can be able to be provided to the one or more reception-authorized units. A scalar value can be assigned to an acceptable or a good status and/or a non-acceptable or a bad status and/or any transition status.

It can be provided that the transmitted items of information are designed for storage in the one or more reception-authorized units, in particular for process optimization of the production system. For example, these items of information can be used as simulation information and/or can form such simulation information.

Additionally or alternatively, it can be provided that the transmitted items of information are designed for storage in the one or more reception-authorized units according to various data categories.

Additionally or alternatively, it can be provided that the transmitted items of information are provided depending on the data category with an information stamp, in particular a release stamp and/or a timestamp and/or a location stamp, to restrict an access to these items of information.

It can be provided that each computing unit comprises at least one communication module, in particular a Bluetooth module, preferably a Bluetooth low-energy module, and/or a Wi-Fi module, and each computing unit is configured and programmed to form a network with every other computing unit for information transmission by means of the at least one communication module.

It is to be understood that the communication module can be alternatively designed. For example, the communication module can be based on: a ZigBee technology, a 6LoWPAN technology, a LoRaWAN technology, a NB-IoT technology, a LTE technology, a 5G technology, a Wi-Sun technology, etc.

It can be provided that one or more of the computing units are configured and programmed as network nodes and/or a hub for information transmission from the processing units to the one or more reception-authorized units by means of the at least one communication module.

It can be provided that one or more of the computing units are configured and programmed as a gateway for information transmission from the processing units to the one or more reception-authorized units by means of the at least one communication module.

It can be provided that the production system comprises a zero trust architecture and/or is operated and/or operable according to a zero trust architecture. In this way, a production security of the products to be produced, in particular biological-pharmaceutical products, for example cell culture and/or cell therapy products, can be and/or become enhanced. For example, it is thus possible to preclude cell cultures from being able to be exchanged and/or personal data from being able to flow out due to external, for example malicious, interventions in the system or the production system. In particular, an area for attack and/or effects of cyber attacks with respect to the production system can be minimized.

It can be provided that the processing units and in particular their computing units are part of the zero trust architecture and/or can be associated or are associated therewith.

It can be provided that the processing units and in particular their computing units are configured and programmed to only permit access authorizations in order to execute a predetermined operation, for example based on a production program and/or a production program packet. The authorizations of users and/or devices and/or units, for example processing units, of the production system can in a certain sense be able to be restricted and/or be restricted to a minimum required level in order to be able to fulfill their respective tasks within the production system. In this way, it is preferably possible to prevent any attackers from being able to utilize, for example, overprivileged accounts and/or devices and/or processing units, for example for a manipulation.

It can be provided that the processing units and in particular their computing units are configured and programmed to continuously and/or discontinuously monitor and/or check a respective identity and/or a respective status of users and/or devices and/or units, for example processing units, of the production system, for example also during running operation. In this way, it can preferably be ensured that no unauthorized and/or compromised actors can obtain and/or retain access to the production system, in particular the network.

It can additionally or alternatively be provided that the at least one control unit and/or one or more separate control units of the production system are configured and programmed to continuously and/or discontinuously monitor and/or check a respective identity and/or a respective status of users and/or devices and/or units, for example processing units, of the production system, for example also during running operation. In this way, it can preferably be ensured that no unauthorized and/or compromised actors can obtain and/or retain access to the production system, in particular the network.

It can be provided that the production system, in particular the processing units, is (are) segmented into multiple network areas. In other words, said network can comprise multiple network areas. The network areas can preferably be isolated from one another in sections or completely. A data traffic or information exchange, for example, between network areas can be strictly supervised and/or a type of data traffic or information exchange can be predefined, so that any deviations therefrom can be detectable. In this way, it can preferably be ensured that a number of potential entry points for attackers is reduced and/or a spread of threats within the network is limited.

It can be provided that an access to resources located within the production system, for example processing units and/or tools and/or consumable materials and/or computing power, can take place on the basis of a preferably strict authentication and/or authorization. Preferably, each access attempt to said resources can be checked before it can be allowable. For example, this can take place on a or the unique item of information, in particular a or the unique item of fingerprint information, and/or a production program packet, as already described herein. In this way, it is preferably possible to prevent potential attackers from being able to access, for example, sensitive and/or critical data, information, and/or systems, for example processing units and/or tools, etc.

High demands on security, quality, and/or compliance of an in particular biological-pharmaceutical production environment, which is considered to be highly regulated and/or sensitive, can be able to be met and/or can be met by means of the zero trust architecture. Preferably, the security situation of the in particular biological-pharmaceutical production environment can be improved and/or business goals in this regard can be reliably protected using the zero trust architecture.

The following description refers in particular to an educt:

An educt can be arranged and/or accommodated in a container, in particular an educt container or a vessel.

The educt container having the educt can be packed by means of packaging.

The educt container having the educt can be able to be introduced into the airlock device of the production system, in particular on an entry airlock space of the airlock device.

The at least one control unit associates the educt container having the educt with a processing unit, in particular a patient box.

The packaging of the educt container can be removable from the educt container in particular at the entry airlock space and/or at a separate unpacking space in the airlock device, in particular by means of one or more unpacking units. This procedure can be comprised in an instruction of a production program packet.

It can preferably be provided that the packaging is cleaned and/or sterilized before the removal by means of a cleaning device of the airlock device or a part of the cleaning device of the production system associated with the airlock device. This procedure can be comprised in an instruction of a production program packet.

It can be provided that the educt container having the educt is movable inside the airlock device by means of an airlock transport device and/or the handling device. This can optionally already take place in the patient box. This procedure can be comprised in an instruction of a production program packet.

By means of the handling device, the educt container having the educt can be transferable to the patient box in or directly adjacent to the airlock device and/or can be transferable to a workpiece carrier, which is arrangeable or is arranged in the patient box. This procedure can be comprised in an instruction of a production program packet.

The patient box which has accommodated the educt container having the educt can define an aseptic processing unit.

The patient box which has accommodated the educt container having the educt can be placed for storage in or on a storage space of the storage device by means of a storage transport device of the storage device. This procedure can be comprised in an instruction of a production program packet.

Additionally or alternatively, this patient box can be provided for one or more treatment procedures, in particular by means of one or more tool units, in particular by means of a tool box, by means of the storage transport device. This procedure can be comprised in an instruction of a production program packet.

The patient box and the tool box can be able to be connected/coupled with one another or can be connected/coupled with one another for one or more treatment procedures on the educt arranged in the receptacle box, wherein for one or more treatment procedures of the treatment procedures, one or more tool units and/or one or more consumable materials can be required, which can be arranged and/or comprised in particular in the tool box. This procedure can be comprised in an instruction of at least one production program packet. In particular, each individual treatment procedure can be comprised in an instruction of at least one single production program packet.

A product can be able to be generated from the educt by means of the treatment procedures, in particular according to the order of the production program packets.

In a certain sense, the educt container held in the patient box can now be referred to as a product container.

The product container having the product which is arranged in the patient box can be movable by means of the storage transport device and/or the airlock transport device and/or handling device to an exit airlock space, in particular to a packing space, of the airlock device. This procedure can be comprised in an instruction of at least one production program packet.

The product container having the product can be able to be packed or can be packed at the exit airlock space, in particular at the packing space, by means of one or multiple packing units in order to be removable or removed subsequently from the airlock device. This procedure can be comprised in an instruction of at least one production program packet.

It is to be understood that for each preceding described procedure, a corresponding item of release information was processed by means of the participating processing units and a corresponding decryption was carried out by means of the participating processing units.

Furthermore, it is to be understood that the production system has been situated in a or the normal status, thus a data connection has existed to the computer system which is associated or can be associated with the production system.

Processing units designed as patient boxes and/or as tool boxes are to be discussed once again hereinafter:

One or more patient boxes and/or tool boxes can be able to be fed via the airlock device to the clean room area.

One or more patient boxes and/or tool boxes can be cleanable and/or sterilizable in the airlock device by means of a cleaning device of the airlock device or a part of the cleaning device of the production system associated with the airlock device.

One or more patient boxes and/or tool boxes can be placed for storage at a storage space of the storage device by means of a storage transport device of the storage device, to which the relevant boxes are transferable by means of an airlock transport device and/or the handling device.

If needed, one or more patient boxes and/or tool boxes can be able to be fed starting from the storage space to an intended use by means of the storage transport device and, optionally, the handling device and/or the airlock transport device.

For example, a patient box and/or a tool box can be able to be equipped or can be equipped with one or more educts and/or one or more tool units and/or one or more consumable materials, for example by means of the handling device, in particular to subsequently be able to be placed for storage or be placed for storage in the storage device or be able to be provided for one or more treatment procedures, wherein in particular multiple patient boxes and/or tool boxes can be provided in this way.

One or more tool carriers can be arrangeable in respective patient boxes and/or tool boxes, for example by means of the handling device, by means of which the tool carriers can be placed for storage and/or provided.

Multiple patient boxes and/or tool boxes can be able to be connected/coupled with one another in order to form an aseptic processing unit to carry out one or more treatment procedures according to the instructions of one or more production program packets.

For example,
a patient box which has accommodated an educt container having an educt,
a tool box which has accommodated the consumable material for one or more treatment procedures, and
a tool box which has accommodated one or more tool units for one or more treatment procedures,

can be able to be connected/coupled or can be connected/coupled with one another, in particular by means of the storage transport device and/or the handling device, in order to form a common aseptic processing unit, in order subsequently to enable one or more treatment procedures to be carried out according to the instructions of one or more production program packets. It is to be understood that the objects can already be accommodated in the relevant boxes according to the instructions of one or more production program packets.

After the treatment procedures are carried out, the boxes can be able to be detached/decoupled or can be detached/decoupled from one another again.

The patient box having the manufactured product is transported as described above to the airlock device by means of the storage transport device.

The tool boxes can be able to be fed to a cleaning device of the production system, by means of which the tool carriers are cleanable and/or sterilizable.

The cleaned and/or sterilized tool boxes can be placed for storage again at storage spaces of the storage device and can be available for further use.

With reference to the preceding description, a fully automated overall system for manufacturing biological-pharmaceutical products for the industrial production of individual batch sizes, in particular individual and/or ultrasmall batch sizes, can be provided by the present invention, in particular having modularly configured and/or autonomously acting components.

In particular a fully automated overall system for manufacturing biological-pharmaceutical products can be provided by the present invention, which has enhanced security in particular with respect to manipulation and/or industrial espionage.

In relation to the typically existing permanently linked individual process steps of conventional production processes of comparable biological-pharmaceutical products, a more efficient system and method can be provided by the present invention. In particular, individual processing steps can be releasable in a tailor-made manner by a rights holder by means of individual release procedures.

A system, i.e. the production system, and a method can be provided by the present invention, by means of which a human operator or user is no longer necessary in the immediate processing environment, by which in particular both a manufacturing efficiency can be increased and also a probability of manual errors can be reduced. In addition, a production optimization can take place on the basis of simulation results, so that a manufacturing efficiency can be further increased.

It can be particularly advantageous that in the present invention, all required systems and/or devices for autonomous operation and associated equipment and/or consumable materials can be present within the production system and furthermore all required items of information for the autonomous operation can be saved and/or processed inside the production system, in particular by means of the at least one control unit, which can preferably be able to be formed by means of the computing units of the processing units.

As the preceding description shows, the invention relates in particular to a production system and a method according to any one of the following sentences, in which furthermore particular embodiments of the invention are described.
1. A production system for manufacturing a product, in particular a biological-pharmaceutical product, which comprises multiple processing units for accommodating and/or treating an educt, wherein each processing unit comprises at least one computing unit.
2. The production system according to sentence 1, characterized in that the production system comprises at least one control unit, which is designed and programmed to associate the educt with one of the multiple processing units on the basis of one of multiple production programs to manufacture one of multiple products that can be manufactured by means of the production system.
3. The production system according to sentence 2, characterized in that a production program comprises multiple, in particular encrypted production program packets, wherein each production program packet contains at least one item of information which refers to another production program packet and/or comprises instructions for accommodating and/or treating an educt.
4. The production system according to sentence 3, characterized in that a defined order of the multiple production program packets forms the production program for manufacturing a product that can be manufactured by means of the production system, wherein in particular the complete order is only determinable from all references with respect to the other production program packets.
5. The production system according to sentence 3 or 4, characterized in that a processing unit is designed and programmed to execute instructions of at least one production program packet, in particular to decrypt and then execute them.
6. The production system according to any one of sentences 3 to 5, characterized in that one or more, in particular all of the production program packets are provided on one processing unit in each case and/or are transmittable to one processing unit in each case, in particular by means of the at least one control unit and/or by means of a computer system which is associated or can be associated with the production system.
7. The production system according to any one of sentences 3 to 6, characterized in that the multiple, in particular encrypted production program packets comprise a starting packet and one or more following packets, wherein each packet contains at least one item of information which refers to a following packet and/or comprises instructions for accommodating and/or treating an educt.
8. The production system according to sentence 7, characterized in that the educt can only be associated with a processing unit which is associated with the starting packet and/or which refers to the starting packet and/or on which the starting packet is stored, or can be associated with a processing unit which the starting packet can be provided with after or with an association of the educt.
9. The production system according to any one of sentences 1 to 8, characterized in that a processing unit is designed and programmed to save one or more cryptographic keys, by means of which production program packets of the production program can be respectively decrypted.
10. The production system according to sentence 9, characterized in that a cryptographic key is stored by means of at least one of the processing units, by means of which a production program packet for starting the production program can be decrypted and/or activated.
11. The production system according to any one of sentences 3 to 10, characterized in that one or more production program packets can be locally decrypted in a processing unit by means of the computing unit of the processing unit, and/or one or more production program packets can be decrypted outside the processing units by means of the at least one control unit and/or by means of a computer system which is associated or can be associated with the production system.
12. The production system according to any one of sentences 9 to 11, characterized in that the one or the multiple cryptographic keys of the processing unit are assignable by means of the at least one control unit and/or by means of a computer system which is associated or can be associated with the production system, and/or the one or the multiple cryptographic keys are integrated inseparably in the processing unit.
13. The production system according to any one of sentences 9 to 12, characterized in that a processing unit comprises a security module, in particular a trusted platform module, for providing one or more cryptographic keys, wherein in particular one or more cryptographic keys for decrypting individual production program packets of the production program are storable by means of the security module.
14. The production system according to any one of sentences 9 to 13, characterized in that a processing unit is designed and programmed to receive an item of release information for a use and/or a provision of the one or more cryptographic keys, and/or the one or more cryptographic keys can only be used and/or provided in a time-limited manner by the processing unit.
15. The production system according to any one of sentences 2 to 14, characterized in that the educt can only be associated with a processing unit and/or a treatment of the educt by means of a processing unit can only be carried out if an item of unique information related to the processing unit, which can be provided by means of the processing unit itself, and a further item of unique information related to the processing unit, which can be detected by means of the at least one control unit, correspond.
16. The production system according to any one of sentences 1 to 15, characterized in that the multiple processing units comprise: one or more patient boxes for accommodating and/or transporting an educt; and/or one or more tool boxes for treating an educt; and/or one or more tool units for treating an educt and/or one or more sensor units for analyzing an educt.
17. The production system according to any one of sentences 2 to 16, characterized in that at least one processing unit is configured to feed an educt into the production system and/or to remove an educt, an at least partially treated educt, and/or a product from the production system, wherein in particular this processing unit is designed and programmed to execute a start, in particular a starting packet, of a production program.
18. The production system according to any one of sentences 1 to 17, characterized in that the production system comprises at least one control unit, in particular the at least one control unit, which can be designed and programmed to determine an integrity of each processing unit, in particular before an educt can be accommodated and/or treated by means of a processing unit according to one of multiple production programs for manufacturing one of multiple products that can be manufactured by means of the production system.
19. The production system according to sentence 18, characterized in that the computing unit of a processing unit is designed and programmed to provide a unique item of information related to this processing unit, and a further unique item of information related to this processing unit is detectable by means of the at least one control unit, wherein the integrity of this processing unit is determinable based on a comparison of these two unique items of information in particular by means of the at least one control unit and in particular is settable and/or confirmed upon correspondence of these two unique items of information.
20. The production system according to sentence 18 or 19, characterized in that a processing unit comprises a security module, in particular a trusted platform module, for providing a unique item of information, in particular an item of fingerprint information about the processing unit, wherein the computing unit of the processing unit is designed and programmed to provide the unique item of information of the at least one control unit to determine the integrity.
21. The production system according to any one of sentences 18 to 20, characterized in that the production system comprises one or more sensor units, by means of which the processing units are in particular visually detectable, and on the basis of this detection a unique item of information with respect to the respective processing units can be provided by means of the at least one control unit to determine the integrity.
22. The production system according to sentence 21, characterized in that the one or more sensor units comprises one or more camera units, by means of which characteristic landmarks of a face, in particular a surface, and/or a geometry of the processing units are visually detectable.
23. The production system according to sentence 21 or 22, characterized in that the production system comprises one or more actuator units, by means of which one or more processing units can be mechanically excited in particular to generate an in particular mechanical oscillation, which is detectable by means of the one or more sensor units and on the basis of which a unique item of information with respect to the respective processing units can be provided by means of the at least one control unit to determine the integrity.
24. The production system according to sentence 23, characterized in that at least one of the one or multiple actuator units is configured to generate a mechanical oscillation and this actuator unit can be arranged on or in one of the one or more processing units and/or can be able to be engaged with one of the one or more processing units to generate the mechanical oscillation at the relevant processing unit.
25. The production system according to sentence 23 or 24, characterized in that the oscillation comprises a frequency range of greater than or equal to 10 Hz and less than or equal to 100 Hz, and/or the oscillation comprises one or more in particular different frequency ranges, which are respectively greater than or equal to 10 Hz and less than or equal to 100 Hz, and/or the oscillation comprises one or more in particular different frequency ranges which follow one another in a specific order.
26. The production system according to any one of sentences 23 to 25, characterized in that at least one of the one or multiple actuator units is configured to generate an acoustic oscillation, in particular a sound wave, and this actuator unit can be arranged on or in one of the one or more processing units and/or can be able to be engaged with one of the one or more processing units to generate the mechanical oscillation, in particular the sound wave, at the relevant processing unit.
27. The production system according to sentence 26, characterized in that the oscillation comprises a frequency range of greater than or equal to 30 Hz and less than or equal to 10 000 Hz, and/or the oscillation comprises one or more in particular different frequency ranges, which are respectively greater than or equal to 30 Hz and less than or equal to 10 000 Hz, and/or the oscillation comprises one or more in particular different frequency ranges which follow one another in a specific order.
28. The production system according to any one of sentences 18 to 27, characterized in that one or more, in particular all processing units comprise at least one receptacle chamber, in particular enclosed by a lateral surface, for accommodating one or more objects, in particular an educt and/or one or more objects for treating the educt, wherein the integrity comprises an internal integrity which relates to the receptacle chamber, and/or comprises an external integrity which relates to an exterior with respect to the receptacle chamber.
29. The production system according to any one of sentences 18 to 28, characterized in that a processing unit is designed and programmed to execute instructions according to at least one production program, in particular to decrypt and then execute them, if its integrity is set and/or confirmed.
30. The production system according to sentence 29, characterized in that a production program comprises multiple, in particular encrypted production program packets, wherein each production program packet contains at least one item of information which refers to another production program packet and/or comprises instructions for accommodating and/or treating an educt, wherein: (a) one or more, in particular all of the production program packets are each provided on one processing unit and can only be executed, in particular can only be executed and decrypted, if the integrity of the relevant processing unit is set and/or confirmed; and/or (b) one or more, in particular all of the production program packets are transmittable to one processing unit in each case, in particular by means of the at least one control unit and/or by means of a computer system which is associated or can be associated with the production system, if the integrity of the relevant processing unit is set and/or confirmed.
31. The production system according to any one of sentences 18 to 30, characterized in that a processing unit is designed and programmed to block an execution and/or a decryption of instructions according to at least one production program if their integrity cannot be set and/or cannot be confirmed.
32. The production system according to any one of sentences 18 to 31, characterized in that a processing unit is designed and programmed to provide a message, in particular an alarm message, in particular to the at least one control unit and/or a computer system which is associated or can be associated with the production system, if their integrity cannot be set and/or cannot be confirmed.
33. The production system according to any one of sentences 1 to 32, characterized in that the at least one computing unit is designed and programmed to determine an integrity of the respective processing unit, in particular before an educt can be accommodated and/or treated by means of this processing unit according to one of multiple production programs for manufacturing one of multiple products that can be manufactured by means of the production system.
34. The production system according to sentence 33, characterized in that the computing unit of a processing unit is designed and programmed to store and/or provide a unique item of information related to this processing unit, and to detect a further unique item of information related to this processing unit, wherein the integrity of this processing unit is determinable based on a comparison of these two unique items of information by means of the computing unit and in particular is settable and/or confirmed upon correspondence of these two unique items of information.
35. The production system according to sentence 34, characterized in that the unique item of information comprises a starting status of the relevant processing unit and a starting timestamp that can be associated with the starting status and the further unique item of information comprises a further status of the relevant processing unit and a further timestamp that can be associated with the further status and is different from the starting timestamp.
36. The production system according to any one of sentences 33 to 35, characterized in that one or more, in particular all processing units comprise at least one receptacle chamber, in particular enclosed by a lateral surface, for accommodating one or more objects, in particular an educt and/or one or more objects for treating the educt, wherein the integrity comprises an internal integrity which relates to the receptacle chamber, and/or comprises an external integrity which relates to an exterior with respect to the receptacle chamber.
37. The production system according to any one of sentences 33 to 36, characterized in that a processing unit comprises one or more sensor units, by means of which a unique item of information and/or a further unique item of information are detectable via the relevant processing unit to determine the integrity, in particular an internal integrity.
38. The production system according to sentence 37, characterized in that a brightness value in and/or on the relevant processing unit is detectable by means of at least one sensor unit and the unique item of information and/or the further unique item of information are able to be provided via the relevant processing unit based on the detectable brightness value to determine the integrity, in particular an internal integrity.
39. The production system according to sentence 37 or 38, characterized in that a processing unit comprises one or more actuator units, by means of which the relevant processing unit can be mechanically excited in particular to generate an in particular mechanical oscillation, which is detectable by means of one or more sensor units and on the basis of which the unique item of information and/or the further unique item of information with respect to the relevant processing unit can be provided to determine the integrity.
40. The production system according to sentence 39, characterized in that the oscillation comprises a frequency range of greater than or equal to 10 Hz and less than or equal to 100 Hz, and/or the oscillation comprises one or more in particular different frequency ranges, which are respectively greater than or equal to 10 Hz and less than or equal to 100 Hz, and/or the oscillation comprises one or more in particular different frequency ranges, which follow one another in a specific order, wherein in particular at least one of the one or more actuator units is designed as a vibration motor, which is fixed in and/or on the relevant processing unit to generate the mechanical oscillation.
41. The production system according to sentence 39 or 40, characterized in that at least one of the one or multiple actuator units is configured to generate an acoustic oscillation, in particular a sound wave, and is fixed in and/or on the relevant processing unit to generate the mechanical oscillation, in particular the sound wave, with respect to the relevant processing unit, wherein in particular: the oscillation comprises a frequency range of greater than or equal to 30 Hz and less than or equal to 10 000 Hz, and/or the oscillation comprises one or more in particular different frequency ranges, which are respectively greater than or equal to 30 Hz and less than or equal to 10 000 Hz, and/or the oscillation comprises one or more in particular different frequency ranges which follow one another in a specific order.
42. The production system according to any one of sentences 37 to 41, characterized in that a processing unit comprises one or more electrically conductive line elements, which are arranged in and/or on the processing unit and/or are arranged inside a lateral surface wall of the processing unit and which form an in particular independent electric circuit, wherein it is detectable by means of one or more sensor units whether the electric circuit is changed with respect to its original status and/or is interrupted, and wherein the item of unique information and/or the further item of unique information about the relevant processing unit can be provided to determine the integrity on the basis of the circuit detection.
43. The production system according to any one of sentences 37 to 42, characterized in that at least one sensor unit can be designed as an inertial measuring unit, by means of which at least one acceleration value, in particular an acceleration vector, and/or at least one rotation rate value, in particular a rotation rate vector, is detectable with respect to the relevant processing unit, and the unique item of information and/or the further unique item of information about the relevant processing unit can be provided based on the detectable acceleration and/or rotation rate values to determine the integrity.
44. The production system according to sentence 43, characterized in that the production system comprises a handling device for moving an object within the production system and/or one or more actuator units, by means of which the relevant processing unit can be mechanically excited to generate an in particular defined movement, which is detectable by means of the inertial measuring unit and on the basis of which the unique item of information and/or the further unique item of information about the relevant processing unit can be provided to determine the integrity.
45. The production system according to any one of sentences 37 to 44, characterized in that a temperature value in and/or on the relevant processing unit is detectable by means of at least one sensor unit and the unique item of information and/or the further unique item of information about the relevant processing unit can be provided based on the detectable temperature value to determine the integrity.
46. The production system according to any one of sentences 37 to 44, characterized in that the relevant processing unit is at least partially visually detectable by means of at least one sensor unit and the unique item of information and/or the further unique item of information about the relevant processing unit can be provided based on the visual detection to determine the integrity, wherein in particular the at least one sensor unit comprises a camera unit, by means of which characteristic landmarks and/or geometries of the relevant processing unit are visually detectable.
47. The production system according to any one of sentences 33 to 46, characterized in that the production system comprises at least one control unit, which is designed and programmed to determine an integrity of each processing unit, in particular before an educt can be accommodated and/or treated by means of a processing unit according to one of multiple production programs to manufacture one of multiple products that can be manufactured by means of the production system, wherein in particular the computing unit of a processing unit is furthermore designed and programmed to provide a unique item of information related to this processing unit, and a further item of unique information related to this processing unit is detectable by means of the at least one control unit, wherein the integrity of this processing unit is determinable based on a comparison of these two items of unique information by means of the at least one control unit and in particular is settable and/or confirmed upon correspondence of these two unique items of information.
48. The production system according to any one of sentences 1 to 47, characterized in that an educt can be accommodated and/or treated by means of a processing unit according to one of multiple production programs for manufacturing one of multiple products that can be manufactured by means of the production system, wherein at least a part of a production program is used in particular exclusively to emulate and/or suggest one or more production program processes, in particular one or more production program processes of a manufacture of a product.
49. The production system according to sentence 48, characterized in that a production program comprises multiple, in particular encrypted production program packets, wherein each production program packet contains at least one item of information which refers to another production program packet and/or comprises instructions for accommodating and/or treating an educt, wherein in particular a defined order of the multiple production program packets forms the production program for manufacturing a product that can be manufactured by means of the production system.
50. The production system according to sentence 48 or 49, characterized in that a production program comprises one or more, in particular encrypted dummy production program packets, wherein each dummy production program packet contains at least one item of information which refers to a production program packet or another dummy production program packet and/or which comprises dummy instructions for emulating and/or suggesting an accommodation and/or a treatment of an educt.
51. The production system according to sentence 50, characterized in that the production system comprises at least one random module, which is executable by means of one or more computing units of the processing units, by means of one at least one control unit of the production system, and/or by means of a computer system that is associated or can be associated with the production system and by means of which one or more dummy production program packets can be integrated in a production program in a random manner, wherein in particular one or more dummy instructions can be associated with each dummy production program packet in a random manner by means of the at least one random module.
52. The production system according to any one of sentences 48 to 51, characterized in that a processing unit is designed and programmed to execute any instructions according to a production program, in particular to decrypt and then execute them.
53. The production system according to any one of sentences 48 to 52, characterized in that a processing unit is designed and programmed to accommodate and/or treat a dummy educt, in order to emulate and/or suggest an accommodation and/or a treatment of an actual educt using the dummy educt.
54. The production system according to any one of sentences 48 to 53, characterized in that the production system comprises one or more concealment chambers for accommodating one or more processing units, wherein an interior of each concealment chamber is optically separated and/or at least largely cannot be seen into from outside the concealment chamber, wherein in particular at least one processing unit can be prompted by means of the at least one part of a production program for emulating and/or suggesting a manufacture of a product in order to be accommodated in the interior of a relevant concealment chamber.
55. The production system according to sentence 54, characterized in that at least one concealment chamber comprises one or more changing units, by means of which objects which are accommodated in and/or by processing units can be rearranged between the relevant processing units which are located inside the concealment chamber.
56. The production system according to sentence 54 or 55, characterized in that at least one concealment chamber comprises one or more storage spaces on which one or more processing units can be accommodated for a random period of time.
57. The production system according to any one of sentences 54 to 56, characterized in that at least one concealment chamber is exclusively configured for emulating and/or suggesting a treatment of an educt.
58. The production system according to any one of sentences 54 to 57, characterized in that at least one concealment chamber comprises one or more sensor units for monitoring the optical separation of the interior from the exterior of the concealment chamber, wherein in particular a brightness value inside the concealment chamber is detectable by means of at least one sensor unit of the concealment chamber.
59. The production system according to any one of sentences 54 to 58 in combination with sentence 50, characterized in that at least one dummy production program packet comprises one or more dummy instructions, which cause one or more processing units to be accommodated in one of the concealment chambers.
60. The production system according to sentence 59, characterized in that at least one dummy production program packet comprises one or more dummy instructions, which cause a rearrangement of objects accommodated in and/or by processing units between the relevant processing units, which are located inside the concealment chamber.
61. The production system according to any one of sentences 1 to 60, characterized in that the production system comprises at least one control unit, which is designed and configured to at least partially store and operate a database and/or to exchange items of information with a database, which is at least partially stored and/or operated on a computer system that is associated or can be associated with the production system, wherein the database contains one or more, in particular unique items of release information for authorizing an accommodation and/or treatment of an educt by means of one of the multiple processing units, wherein the database is changeable by a retrieval of at least one of the items of release information for one of the multiple processing units so that this release information subsequently can no longer be provided in the original form for retrieval.
62. The production system according to sentence 61, characterized in that the database is configured and programmed to receive and store one or more new items of release information exclusively from outside the production system.
63. The production system according to sentence 61 or 62, characterized in that the database is configured and programmed to be storable and/or operable exclusively on the computer system that is associated or can be associated with the production system, or to be able to be synchronized with a parallel database, which is storable and/or operable exclusively on the computer system that is associated or can be associated with the production system.
64. The production system according to any one of sentences 61 to 63, characterized in that each computing unit of each processing unit is configured and programmed to receive and store one or more items of release information, wherein each item of release information received by a computing unit can be compared with each item of release information already stored on a computing unit to avoid a double use of the received release information in the production system, wherein in particular each computing unit is designed and programmed to at least partially or completely store and operate an in particular encrypted decentralized database, preferably an in particular encrypted distributed ledger, in order to store the one or more receivable or received items of release information.
65. The production system according to any one of sentences 61 to 64, characterized in that one of multiple products can be manufactured according to one of multiple production programs by means of the production system, wherein a production program comprises multiple production program packets and each production program packet contains at least one item of information which comprises instructions for accommodating and/or treating an educt, wherein each production program packet can be uniquely associated with at least one of the items of release information to authorize an in particular one-time execution of this production program packet by means of at least one processing unit, wherein in particular each production program can only be initialized and/or started by means of the production system if all production program packets of the production program and/or all items of release information associated with the production program packets can be provided or are provided.
66. The production system according to any one of sentences 61 to 65, characterized in that one or more items of release information are receivable and storable by means of at least one processing unit for a time-delayed authorization and execution of an accommodation and/or a treatment of an educt and/or one or more items of release information are receivable by means of at least one processing unit in real time for an immediate authorization and execution of an accommodation and/or a treatment of an educt.
67. The production system according to any one of sentences 61 to 66, characterized in that the database can be encrypted or is encrypted and/or each item of release information in the database can be encrypted or is encrypted.
68. The production system according to any one of sentences 61 to 67, characterized in that one of multiple products can be manufactured according to one of multiple production programs by means of the production system, wherein at least a part of a production program is in particular exclusively used to emulate and/or suggest a manufacture of a product and this at least one part of a production program can be authorized for execution by means of one or more items of release information.
69. The production system according to any one of sentences 61 to 68, characterized in that one or more items of release information can be able to be retrieved from the database as bundled release information for authorizing an overall process for manufacturing a product, in particular by means of one or more processing units, and/or can be provided by means of the database.
70. The production system according to any one of sentences 61 to 64 or 66 to 69 in combination with sentence 65, characterized in that an item of release information for each individual production program packet can be retrieved individually from the database, in particular by means of a processing unit, and/or can be provided by means of the database, and/or one or more items of release information for production program packets which can be associated can be retrieved together from the database, in particular by means of one or more processing units, and/or can be provided by means of the database.
71. The production system according to any one of sentences 61 to 70, characterized in that the database is configured and programmed to associate one or more, in particular all items of release information with a respective item of confirmation information, which is storable in the database for each item of release information, in particular by a database access authorized party, wherein each item of release information can only be provided if an associated item of confirmation information is stored.
72. The production system according to any one of sentences 61 to 71, characterized in that the database is configured and programmed to contain and provide various types of release information, wherein in particular a type of a release corresponds with a type of a processing unit and/or with a type of a part, in particular a production program packet, of a production program for manufacturing a product.
73. The production system according to sentence 72, characterized in that each item of release information comprises an information stamp, in particular a mode information stamp, by means of which the respective item of release information can be associated with a production mode of the production system for manufacturing a product and/or a simulation mode of the production system for simulating the operation of the production system and/or in particular the manufacturing of a product.
74. The production system according to sentence 72 or 73, characterized in that each item of release information comprises an information stamp, in particular a number information stamp, by means of which an in particular maximum repeatability of an accommodation and/or a treatment of an educt is settable, which can be authorized by means of the relevant release information.
75. The production system according to any one of sentences 61 to 74, characterized in that the at least one control unit is designed and programmed to be connectable or connected for data exchange to the computer system that is associated or can be associated with the production system to determine an existence of this data connection, wherein the production system and/or the one or more, in particular all processing units are reversibly transferable depending on the data connection into a normal status for unrestricted operation, into a reserve status for chronologically and/or functionally restricted operation, and/or into a pausing status for idled operation.
76. The production system according to any one of sentences 1 to 75, characterized in that each computing unit is designed and programmed to at least partially or completely store and operate an in particular encrypted decentralized database, preferably an in particular encrypted distributed ledger, wherein each decentralized database is a redundant copy of each of the other decentralized databases, wherein each computing unit is designed and programmed to validate an operation of the respective decentralized database on each of the other computing units and to update its own decentralized database in response to a validation, wherein each decentralized database is designed and programmed to comprise one or more, in particular unique items of information about an accommodation and/or a treatment of an educt by each processing unit.
77. The production system according to sentence 76, characterized in that each processing unit, in particular the computing unit of each processing unit, comprises a security module, in particular a trusted platform module, for providing one or more cryptographic keys.
78. The production system according to sentence 76 or 77, characterized in that each decentralized database is able to be encrypted or is encrypted by means of a cryptographic key, which can only be associated with and/or provided to the processing unit on which the decentralized database is storable or stored.
79. The production system according to any one of sentences 76 to 78, characterized in that one of multiple products is able to be manufactured according to one of multiple production programs by means of the production system, wherein a production program comprises multiple production program packets and each production program packet contains at least one item of information which comprises instructions for accommodating and/or treating an educt, which are executable by means of at least one processing unit.
80. The production system according to sentence 79, characterized in that each computing unit is configured and programmed to store each execution of a production program packet in the decentralized database, in particular depending on the validation by the other computing units.
81. The production system according to sentence 79 or 80, characterized in that each computing unit is configured and programmed to execute a production program packet only if this production program packet is not stored as already executed in the decentralized database.
82. The production system according to any one of sentences 76 to 81, characterized in that each computing unit is configured and programmed to receive one or more, in particular unique items of release information for authorizing an accommodation and/or treatment of an educt from at least one control unit of the production system and/or a computer system that is associated or can be associated with the production system and to store the one or more items of release information in its own decentralized database.
83. The production system according to sentence 82, characterized in that each computing unit is configured and programmed to compare each received item of release information with each item of release information already stored in the decentralized database to avoid a double use of the received release information in the production system, in particular depending on the validation by the other computing units.
84. The production system according to sentence 82 or 83, characterized in that each item of release information comprises an information stamp, in particular a number information stamp, by means of which an in particular maximum repeatability of an accommodation and/or treatment of an educt is settable, which can be authorized by means of the relevant release information, wherein in particular the information stamp, in particular the number information stamp, of each item of release information can be updated in the decentralized database depending on an authorized execution of an accommodation and/or a treatment of an educt.
85. The production system according to any one of sentences 79 to 81, in combination with any one of sentences 82 to 84, characterized in that at least one of the items of release information can be uniquely associated with each production program packet to authorize an execution of this production program packet by means of at least one processing unit.
86. The production system according to sentence 85, characterized in that each computing unit is designed and programmed to execute the instructions of a production program packet only if an item of release information associated with this production program packet is provided to the computing unit and in particular this production program packet and/or this release information is stored in the decentralized database as not yet already executed.
87. The production system according to sentence 85 or 86, characterized in that each computing unit is designed and programmed to execute the instructions of a production program packet only if a maximum repeatability of an execution of this production program packet is stored as not yet reached according to the release information in the decentralized database.
88. The production system according to any one of sentences 76 to 87, characterized in that the production system comprises one or more further computer systems and/or is connectable or connected to such further computer systems, wherein each of the further computer systems is arranged in a different location with respect to a remainder of the production system, wherein the decentralized database is stored and operated on each of the further computer systems, and wherein each of the further computer systems is designed and programmed to validate an operation of the respective decentralized databases on each of the other computer systems and/or computing units and to update its own decentralized database in response to a validation.
89. The production system according to any one of sentences 1 to 88, characterized in that the production system comprises at least one control unit, which is designed and programmed to be connectable or connected for data exchange to a computer system that is associated or can be associated with the production system to determine an existence of this data connection.
90. The production system according to sentence 89, characterized in that the production system and/or the one or more, in particular all processing units are reversibly transferable depending on the data connection into a normal status for unrestricted operation, into a reserve status for chronologically and/or functionally restricted operation, and/or into a pausing status for idled operation.
91. The production system according to sentence 89 or 90, characterized in that the at least one control unit and/or one or more, in particular each of the computing units are designed and programmed to determine the existence of the data connection to the computer system, in particular continuously or at regular time intervals.
92. The production system according to any one of sentences 89 to 91, characterized in that the production system is only unrestrictedly operable to manufacture a product, in particular the processing units to accommodate and/or treat an educt, upon the existence of the data connection.
93. The production system according to any one of sentences 89 to 92, characterized in that if the data connection does not exist, the production system for manufacturing a product, in particular the processing units for accommodating and/or treating an educt, is transferable into a reserve status, in which the manufacturing of a product, in particular the accommodation and/or the treatment of an educt, is chronologically and/or functionally restricted.
94. The production system according to any one of sentences 90 to 93, characterized in that the at least one control unit and/or one or more, in particular each of the computing units are designed and programmed to store an information stamp, in particular a timestamp, upon transfer into the reserve status and to stop an unrestricted operation of the production system and/or the one or more, in particular all processing units after a predefined period of time from this information stamp, in particular this timestamp, and in particular to transfer the production system and/or the relevant processing units into the pausing status.
95. The production system according to any one of sentences 90 to 94, characterized in that the at least one control unit and/or one or more, in particular each of the computing units are designed and programmed, upon transfer into the reserve status, to only still execute a predefined part of a production program for manufacturing a product.
96. The production system according to any one of sentences 90 to 95, characterized in that the at least one control unit and/or one or more, in particular each of the computing units can be designed and programmed, upon passing into the pausing status, to modify and/or supplement a production program for manufacturing a product using one or more production program packets to place in storage an educt and/or an at least partially treated educt and/or a product, in particular as long as the pausing status exists.
97. The production system according to any one of sentences 89 to 96, characterized in that the production system comprises the computer system, wherein the computer system can be arranged in a different location with respect to a remainder of the production system, wherein in particular the computer system comprises a server to which the at least one control unit is connectable or connected for data exchange.
98. The production system according to any one of sentences 1 to 97, characterized in that the multiple processing units are used to accommodate and/or treat an educt according to one of multiple production programs for manufacturing one of multiple products that can be manufactured by means of the production system, and that the production system comprises a simulation device, which is designed and programmed to simulate the operation of the production system and/or in particular the manufacturing of a product based on at least one production program and to provide a simulation result corresponding thereto, wherein the simulation device is partially or completely formed by one or more, in particular all computing units of the respective processing units.
99. The production system according to sentence 98, characterized in that the simulation device is designed and programmed to detect system parameters of the production system during a manufacture of a product and/or to detect accommodation procedure measured values and/or treatment procedure measured values of the processing units, in particular operating parameters of the processing units, and store them as simulation information and save them to carry out a simulation, wherein in particular the simulation of an operation of the production system and/or a manufacture of a product can be carried out on the basis of this simulation information by means of the simulation device.
100. The production system according to sentence 98 or 99, characterized in that the production system comprises one or more sensor units, which are at least partially associated with the processing units and/or form a part of the processing units, wherein the simulation device is designed and programmed to carry out the simulation on the basis of items of simulation information detectable or detected by means of these sensor units.
101. The production system according to sentence 99 or 100, characterized in that an item of simulation information comprises at least one of the following: time parameters and/or time measured values of the production system, in particular the processing units, climate parameters and/or climate measured values in and/or around the production system, in particular in and/or around the processing units, and/or operating parameters and/or operating measured values of the production system, in particular the processing units, wherein in particular: (a) time parameters and/or time measured values comprise: pumping periods of processing units configured as pumping devices and/or transport periods of processing units and/or sterilization times of processing units configured as sterilization devices and/or cleaning times of processing units configured as cleaning devices and/or waiting times within the production system and/or unpacking/packing times of processing units configured as unpacking/packing devices and/or loading/unloading times of processing units and/or fitting times of processing units configured as tool units; and/or (b) climate parameters and/or climate measured values comprise: temperature values and/or ambient humidity values and/or gas composition values; and/or (c) operating parameters and/or operating measured values comprise: temperature parameters of processing units configured as temperature control devices and/or fill levels of storage devices.
102. The production system according to any one of sentences 98 to 101, characterized in that the part of a production program can be able to be simulated by means of the computing units forming the simulation device which is to be executed by means of the associated processing units for manufacturing a product, wherein in particular a production program comprises multiple, in particular encrypted production program packets and each production program packet contains at least one item of information which comprises instructions for accommodating and/or treating an educt, which are executable by means of at least one processing unit, and one or more production program packets form said part of the production program to be simulated.
103. The production system according to any one of sentences 98 to 102, characterized in that a production program comprises multiple, in particular encrypted production program packets and each production program packet contains at least one item of information, which comprises instructions for accommodating and/or treating an educt, which are executable by means of at least one processing unit, and the simulation is executable based on one or more production program packets, wherein in particular the one or more production program packets are designed and programmed both for a manufacture of a product and also for simulating the operation of the production system and/or the manufacture of the product.
104. The production system according to sentence 102 or 103, characterized in that a processing unit is designed and programmed to save one or more cryptographic keys, by means of which production program packets of the production program for the simulation can be respectively decrypted.
105. The production system according to any one of sentences 102 to 104, characterized in that each production program packet is releasable for execution, by means of an item of release information receivable by the processing unit, for the simulation of the operation of the production system and/or a manufacture of a product, wherein in particular each item of release information comprises an information stamp, in particular a mode information stamp, by means of which the respective release information and/or the respective production program packet can be associated with a production mode of the production system for manufacturing a product and/or a simulation mode of the production system for simulating the operation of the production system and/or the manufacturing of a product.
106. The production system according to any one of sentences 98 to 105, characterized in that each processing unit is designed and programmed to be operable in a production mode of the production system to manufacture a product and/or a simulation mode of the production system to simulate the operation of the production system and/or the manufacturing of a product.
107. The production system according to sentence 106, characterized in that one or more processing units are designed and programmed to simultaneously simulate one or more production program packets for at least one processing unit in the production mode and in particular to generate items of simulation information in this case.
108. The production system according to any one of sentences 102 to 107, characterized in that each production program packet contains at least one item of information which refers to another production program packet and/or comprises instructions for accommodating and/or treating an educt, wherein in particular a defined order of the multiple production program packets forms the production program for manufacturing a product that can be manufactured by means of the production system.
109. The production system according to sentence 108, characterized in that each reference to another production program packet comprises an information stamp, in particular a mode information stamp, by means of which the respective item of release information can be associated with a production mode of the production system for manufacturing a product and/or a simulation mode of the production system for simulating the operation of the production system and/or the manufacturing of a product.
110. The production system according to any one of sentences 98 to 109, characterized in that the production system comprises at least one control unit, by means of which an in particular logistical utilization and/or occupancy of the production system can be provided based on the simulation result.
111. The production system according to any one of sentences 98 to 110, characterized in that the production system comprises at least one control unit, by means of which, based on the simulation result, a prediction of a chronological production sequence of one or multiple production programs can be prepared and/or, based on the simulation result, the processing units required and/or intended for carrying out the treatment procedures and/or consumable materials to be provided in the production system can be associated chronologically and/or positionally.
112. The production system according to any one of sentences 1 to 111, characterized in that the multiple processing units are used to accommodate and/or treat an educt according to one of multiple production programs for manufacturing one of multiple products that can be manufactured by the production system, and that the production system comprises a simulation device, which is designed and programmed to simulate the operation of the production system and/or in particular the manufacturing of a product based on at least one production program and to provide a simulation result corresponding thereto, wherein the simulation device is partially or completely formed by one or more, in particular all computing units of the respective processing units, and that the production system furthermore comprises at least one control unit, which is designed and programmed to at least partially store and operate a database and/or to exchange items of information with a database, which is at least partially stored and/or operated on a computer system that is associated or can be associated with the production system, wherein the database contains one or more, in particular unique items of release information for authorizing a simulation of an accommodation and/or a treatment of an educt by means of one of the multiple processing units, wherein the database is changeable by retrieval of at least one of the items of release information for a simulation by means of one of the multiple processing units, so that this item of release information can subsequently no longer be provided in the original form for retrieval.
113. The production system according to sentence 112, characterized in that the database is configured and programmed to receive and store one or more new items of release information for authorizing a simulation exclusively from outside the production system.
114. The production system according to sentence 112 or 113, characterized in that the database is configured and programmed to be storable and/or operable exclusively on the computer system that is associated or can be associated with the production system, or to be able to be synchronized with a parallel database, which is storable and/or operable exclusively on the computer system that is associated or can be associated with the production system.
115. The production system according to any one of sentences 112 to 114, characterized in that each computing unit of each processing unit is configured and programmed to receive and store one or more items of release information, wherein each item of release information received by a computing unit can be compared with each item of release information already stored on a computing unit to avoid a double use of the received release information in the production system, wherein in particular each computing unit is designed and programmed to at least partially or completely store and operate an in particular encrypted decentralized database, preferably an in particular encrypted distributed ledger, in order to store the one or more receivable or received items of release information.
116. The production system according to any one of sentences 112 to 115, characterized in that a production program comprises multiple production program packets and each production program packet contains at least one item of information which comprises instructions for accommodating and/or treating an educt, wherein each production program packet can be uniquely associated with at least one of the items of release information to authorize an in particular one-time simulation and/or an in particular one-time execution of this production program packet by means of at least one processing unit, wherein in particular a simulation can only be initialized and/or started by means of the simulation device if all production program packets of the production program and/or all items of release information associated with the production program packets can be provided.
117. The production system according to any one of sentences 112 to 116, characterized in that one or more items of release information are receivable and storable by means of at least one processing unit for a time-delayed authorization and execution of a simulation of an accommodation and/or a treatment of an educt and/or one or more items of release information are receivable by means of at least one processing unit in real time for an immediate authorization and execution of a simulation of an accommodation and/or a treatment of an educt.
118. The production system according to any one of sentences 112 to 117, characterized in that the database can be encrypted or is encrypted and/or each item of release information in the database can be encrypted or is encrypted.
119. The production system according to any one of sentences 112 to 118, characterized in that one of multiple products can be manufactured according to one of multiple production programs by means of the production system, wherein at least a part of a production program is in particular exclusively used to emulate and/or suggest a manufacture of a product and this at least one part of a production program can be authorized for simulation and/or execution by means of one or more items of release information.
120. The production system according to any one of sentences 112 to 119, characterized in that one or more items of release information are retrievable from the database as bundled release information for authorizing a simulation of the operation of the entire production system and/or a simulation of an overall process for manufacturing a product, in particular can be provided by means of one or more processing units and/or by means of the database.
121. The production system according to any one of sentences 112 to 115 or 117 to 120 in combination with sentence 116, characterized in that an item of release information for each individual production program packet can be retrieved individually from the database, in particular by means of a processing unit, and/or can be provided by means of the database, and/or one or more items of release information for production program packets which can be associated can be retrieved together from the database, in particular by means of one or more processing units, and/or can be provided by means of the database.
122. The production system according to any one of sentences 112 to 121, characterized in that the database is configured and programmed to associate one or more, in particular all items of release information with a respective item of confirmation information, which is storable in the database for each item of release information, in particular by a database access authorized party, wherein each item of release information can only be provided if an associated item of confirmation information is stored.
123. The production system according to any one of sentences 112 to 122, characterized in that the database is configured and programmed to contain and provide various types of release information, wherein in particular a type of a release corresponds with a type of a processing unit and/or with a type of a part, in particular a production program packet, of a production program for manufacturing a product.
124. The production system according to sentence 123, characterized in that each item of release information comprises an information stamp, in particular a mode information stamp, by means of which the respective item of release information can be associated with a production mode of the production system for manufacturing a product and/or a simulation mode of the production system for simulating the operation of the production system and/or the manufacturing of a product.
125. The production system according to sentence 123 or 124, characterized in that each item of release information comprises an information stamp, in particular a number information stamp, by means of which an in particular maximum repeatability of a simulation of an accommodation and/or a treatment of an educt is settable, which can be authorized by means of the relevant release information.
126. The production system according to any one of sentences 112 to 125, characterized in that the at least one control unit is designed and programmed to be connectable or connected for data exchange to the computer system that is associated or can be associated with the production system to determine an existence of this data connection, wherein the production system and/or the one or more, in particular all processing units are reversibly transferable depending on the data connection into a normal status for unrestricted operation, into a reserve status for chronologically and/or functionally restricted operation, and/or into a pausing status for idled operation, wherein in particular a simulation can be carried out depending on the respective status.
127. The production system according to any one of sentences 1 to 126, characterized in that the multiple processing units are used to accommodate and/or treat an educt according to one of multiple production programs for manufacturing one of multiple products that can be manufactured by means of the production system, and each processing unit comprises one or more sensor units, wherein each sensor unit is configured to detect at least one measured value with respect to the accommodation and/or the treatment of the educt and/or with respect to the processing unit itself and to provide this measured value to the computing unit for transmission to one or more reception-authorized units in encrypted and/or abstracted form.
128. The production system according to sentence 127, characterized in that the one or more reception-authorized units comprise one or more other processing units and/or at least one higher-order control unit of the production system and/or a computer system which is associated or can be associated with the production system.
129. The production system according to sentence 127 or 128, characterized in that an item of information which represents the at least one detectable measured value is modifiable by the sensor unit itself and the one or more reception-authorized units are designed and programmed to interpret the modified information.
130. The production system according to any one of sentences 127 to 129, characterized in that an item of information which represents the at least one detectable measured value is modifiable by means of the computing unit and the one or more reception-authorized units are designed and programmed to interpret the modified information.
131. The production system according to sentence 129 or 130, characterized in that the information is modifiable in an encrypted manner and/or in an abstracting manner and the information can be decrypted and/or made specific by means of the one or more reception-authorized units.
132. The production system according to any one of sentences 129 to 131, characterized in that each computing unit is configured and programmed to provide the information only as modified information for transmission to the one or multiple reception-authorized units.
133. The production system according to any one of sentences 127 to 132, characterized in that each computing unit is configured and programmed to assess the at least one detectable measured value according to parameter ranges storable or stored in the computing unit and subsequently to provide only a result of the assessment of the at least one detectable measured value for transmission to one or more reception-authorized units.
134. The production system according to any one of sentences 127 to 133, characterized in that the transmitted items of information are designed for storage in the one or more reception-authorized units, in particular for process optimization of the production system.
135. The production system according to any one of sentences 127 to 134, characterized in that the transmitted items of information are designed for storage in the one or more reception-authorized units according to various data categories.
136. The production system according to sentence 135, characterized in that the transmitted items of information are provided with an information stamp, in particular a release stamp, according to the data category for restriction of an access to these items of information.
137. The production system according to any one of sentences 127 to 136, characterized in that each computing unit comprises at least one communication module, in particular a Bluetooth module, preferably a Bluetooth low-energy module, and/or a Wi-Fi module, and each computing unit is configured and programmed to form a network with every other computing unit for information transmission by means of the at least one communication module.
138. The production system according to sentence 137, characterized in that one or more of the computing units are configured and programmed as network nodes and/or a hub for information transmission from the processing units to the one or more reception-authorized units by means of the at least one communication module.
139. The production system according to sentence 137 or 138, characterized in that one or more of the computing units are configured and programmed as a gateway for information transmission from the processing units to the one or more reception-authorized units by means of the at least one communication module.
140. A method for controlling a production system for manufacturing a product, in particular a biological-pharmaceutical product, in particular a production system according to any one of sentences 1 to 139, wherein the method comprises at least the following: providing one or more processing units for accommodating and/or treating an educt.
141. The method according to sentence 140, characterized in that the method furthermore comprises the following:
   determining an in particular encrypted production program packet of a production program, in particular to manufacture a product that can be manufactured by means of the production system, and one of the processing units in order to execute this production program packet, based on a production program packet of the production program which precedes the production program packet and is in particular encrypted;
   activating the determined production program packet on the determined processing unit in order to decrypt the determined production program packet and then to control the processing unit to carry out one or more treatment steps on the educt based on the decrypted production program packet; and
   determining a production program packet of the production program which follows the decrypted production program packet and is in particular encrypted and a following processing unit in order to execute this following production program packet, based on the decrypted production program packet.
142. The method according to sentence 140 or 141, characterized in that the method furthermore comprises the following:
   providing a data connection from a computer system which is associated or can be associated with the production system to the production system and/or one or more, in particular all processing units;
   determining whether the data connection exists by means of at least one control unit of the production system and/or at least one computing unit of at least one processing unit; and
   controlling the production system and/or the one or more, in particular all processing units depending on the data connection.
143. The method according to sentence 142, characterized in that the control comprises a reversible transfer of the production system and/or the one or more, in particular all processing units into a normal status for unrestricted operation, into a reserve status for chronologically and/or functionally restricted operation, and/or into a pausing status for idled operation, in particular based on the existence of the data connection.
144. The method according to sentence 143, characterized in that the at least one control unit and/or one or more, in particular each of the computing units store an information stamp, in particular a timestamp, upon transfer into the reserve status and stop an unrestricted operation of the production system and/or the one or more, in particular all processing units after a predefined period of time from this information stamp, in particular this timestamp, and in particular transfer the production system and/or the relevant processing units into the pausing status.
145. The method according to sentence 143 or 144, characterized in that the at least one control unit and/or one or more, in particular each of the computing units, upon transfer into the reserve status, only still execute a predefined part of a production program for manufacturing a product.
146. The method according to any one of sentences 143 to 145, characterized in that the at least one control unit and/or one or more, in particular each of the computing units, upon passing into the pausing status, modifies and/or supplements a production program for manufacturing a product using one or more production program packets to place in storage an educt and/or an at least partially treated educt and/or a product, in particular as long as the pausing status exists.

Further preferred features and/or advantages of the present invention are the subject matter of the following description and the illustration in the drawings of exemplary embodiments.

### BRIEF DESCRIPTION OF THE DRAWINGS

FIG. 1 shows a schematic perspective representation of a production system according to an exemplary embodiment of the present invention,

FIG. 2 shows a schematic perspective representation of a part of the production system shown in FIG. 1,

FIG. 3 shows a schematic top view representation of a part of the production system shown in FIG. 1,

FIG. 4 shows a schematic top view representation of a part of the production system shown in FIG. 1,

FIG. 5 shows a schematic top view representation of the production system shown in FIG. 1,

FIG. 6 shows a schematic top view representation of the production system shown in FIG. 1,

FIG. 7 shows a schematic top view representation of the production system shown in FIG. 1,

FIG. 8 shows a schematic top view representation of a part of the production system shown in FIG. 1,

FIG. 9 shows a schematic perspective representation of a part of the production system shown in FIG. 1,

FIG. 10 shows a schematic top view representation of a part of the production system shown in FIG. 1,

FIG. 11 shows a schematic top view representation of a part of the production system shown in FIG. 1,

FIG. 12 shows a schematic perspective representation of a production system according to a further exemplary embodiment of the present invention,

FIG. 13 shows a schematic perspective representation of a production system according to a further exemplary embodiment of the present invention,

FIG. 14 shows a schematic perspective representation of a production system according to an exemplary embodiment of the present invention, and

FIG. 15 shows a method according to an exemplary embodiment of the present invention.

Identical or functionally-equivalent elements are provided with the same reference signs in all figures.

### DETAILED DESCRIPTION OF THE DRAWINGS

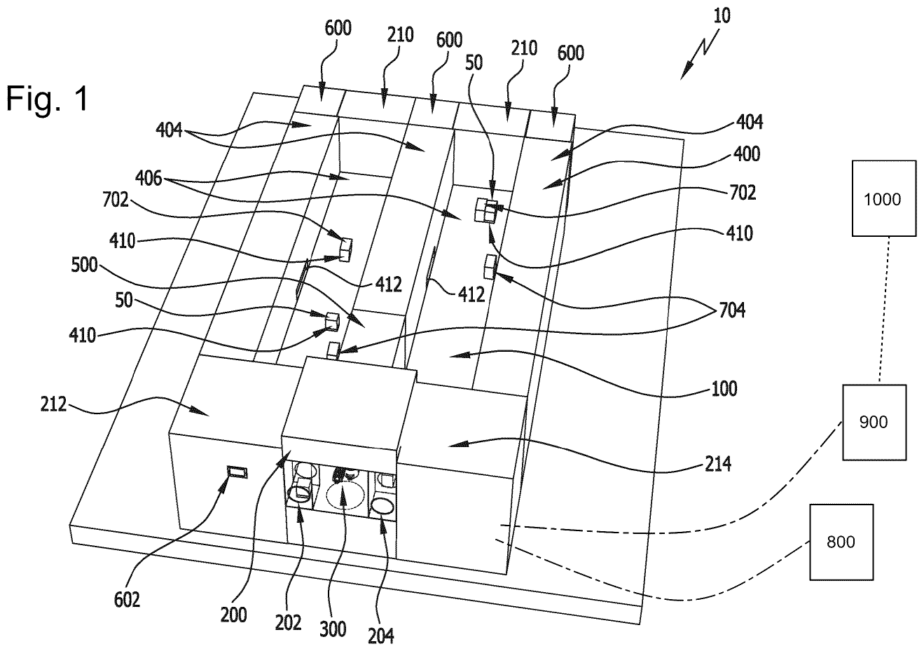

FIG. 1 shows a schematic perspective representation of a production system 10 according to an exemplary embodiment of the present invention.

With reference to FIG. 1, a production system 10, in particular for manufacturing biological-pharmaceutical products 20, comprises multiple processing units for accommodating and/or treating an educt 40.

In the present exemplary embodiment, multiple processing units are designed as workpiece carriers 30, in particular as workpiece carriers 30 configured as patient boxes 50, for accommodating and/or transporting an educt 40 and/or as patient boxes 50, in particular for accommodating and/or transporting an educt 40. In other words, workpiece carriers 30 configured as patient boxes 50 can be patient boxes 30, 50.

Furthermore, in the present exemplary embodiment, multiple processing units are designed as tool boxes 702, 704, in particular for treating an educt 40.

A tool box 702, 704, which is described in the present description in particular in conjunction with tool units, is identified by the reference sign 702, and a tool box 702, 704, which is described in the present description in particular in conjunction with consumable substances and/or consumable materials, is identified by the reference sign 704. It is to be understood that a tool box 702, 704 can be described both with regard to tool units and also consumable substances and/or consumable material and can be identified by both reference signs 702, 704.

Each processing unit comprises a computing unit.

That is to say: in particular each patient box 30, 50 and each tool box 702, 704 comprises a respective computing unit.

Furthermore, the production system 10 shown in FIG. 1 comprises:
a clean room area 100;
an airlock device 200 for feeding an object from the surroundings of the production system 10 into the clean room area 100 and/or for removing an object from the clean room area 100;
a handling device 300 for moving the object within the clean room area 100; and
a storage device 400 arranged inside the clean room area, which comprises multiple storage spaces 402 for accommodating multiple objects.

In the present exemplary embodiment it is provided in particular that the handling device 300 is designed as a processing unit or a processing unit forms the handling device 300, and that a storage space 402 is designed as a processing unit or a processing unit forms a storage space 402.

The biological-pharmaceutical product 20 can be intended for a personalized therapy and can therefore be a personalized therapeutic.

The production system 10 comprises a housing (not shown in the figures) for delimiting the clean room area 100 toward the surroundings of the production system 10.

Therefore, the airlock device 200 is configured to feed an object from the surroundings of the production system 10 via the or through the housing into the clean room area 100 and/or for removing an object from the clean room area 100 via the or through the housing.

The clean room area 100 is designed and/or classified as described in the application DE 10 2021 207 738.2, to which reference is hereby made.

The production system 10 is furthermore connected to at least one electric power source (not shown in the figures), which is arranged in the surroundings of the production system 10, for a power supply.

As can be seen in FIG. 1, the storage device 400 comprises multiple storage racks 404, three by way of example here, which each comprise multiple storage spaces 402.

The storage device 400 furthermore comprises a storage transport device 406 for transporting objects to the storage spaces 402, in particular the storage racks 404, and/or away from the storage spaces 402, in particular the storage racks 404.

In particular, by means of the storage transport device 406 of the storage device 400, educts and/or workpiece carriers and/or tool units and/or consumable materials and/or processing units, for example patient boxes 30, 50 and/or tool boxes 702, 704, can be placed in storage in the storage spaces 402 of the storage racks 404 and/or can be removed therefrom.

The storage transport device 406 is a device different from the handling device 300.

It is provided in particular in the present exemplary embodiment that the storage transport device 406 is designed as processing units or multiple processing units form the storage transport device 406.

The storage transport device 406 schematically shown in FIG. 1 comprises one or more transport vehicles 410 freely moving in the clean room area 100. In other words, it can be provided that the transport vehicles 410 freely moving in the clean room area 100 are transport vehicles which can be actuated and freely moved as needed in the clean room area 100, so that they are respectively free moving as needed upon a corresponding actuation.

It is provided in particular in the present exemplary embodiment that a transport vehicle 410 is designed as a processing unit or a processing unit forms a transport vehicle 410.

By means of the transport vehicles 410, the storage racks 404 of the storage device 400 are accessible in particular for storage placement and removal procedures and for treatment procedures and/or maintenance procedures.

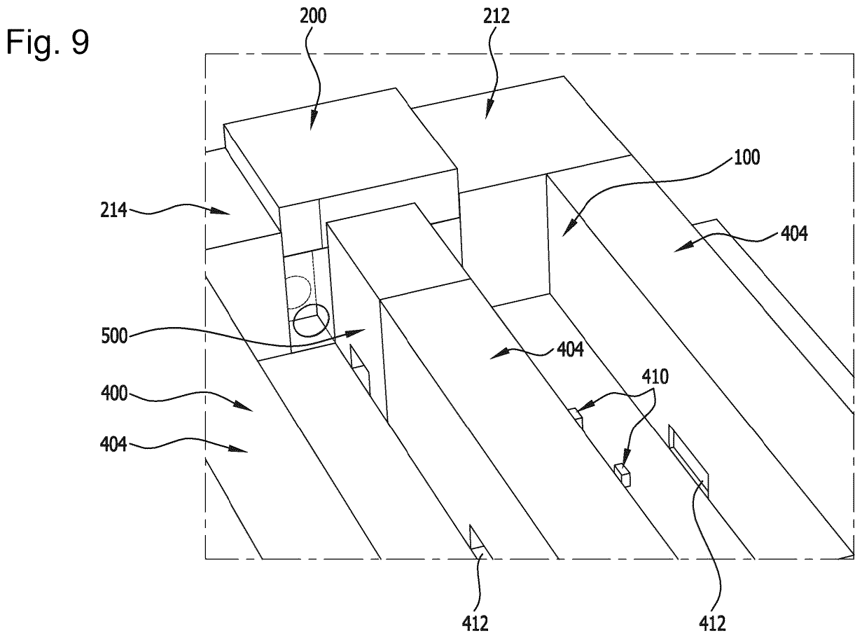

The storage racks 404, in particular the storage spaces 402 of the storage racks 404, are accessible by the transport vehicles 410 via one or more storage rack airlocks 412 for storage placement and removal procedures and for treatment procedures and/or maintenance procedures (cf. FIG. 9).

An arbitrary arrangement of the storage racks 404 is possible due to the use of the freely moving transport vehicles 410. The substantially parallel arrangement of the storage racks 404 shown in FIG. 1 and the figures referring thereto is shown solely by way of example for explanatory purposes.

The production system 10 furthermore comprises a cleaning device 500, by means of which one or more patient boxes 30, 50 and/or one or more tool boxes 702, 704 and/or one or more tools or tool units and/or one or more consumable materials are cleanable and/or sterilizable, in particular by means of irradiation and/or gassing.

The production system 10 moreover comprises an unpacking device 212 for unpacking objects to be fed to the clean room area 100 and a packing device 214 for packing objects to be removed from the clean room area 100, for example for packing the manufactured product 20.

The unpacking device 212 and the packing device 214 are integrated in or attached to the airlock device 200, in particular in the present case integrated or attached laterally arranged.

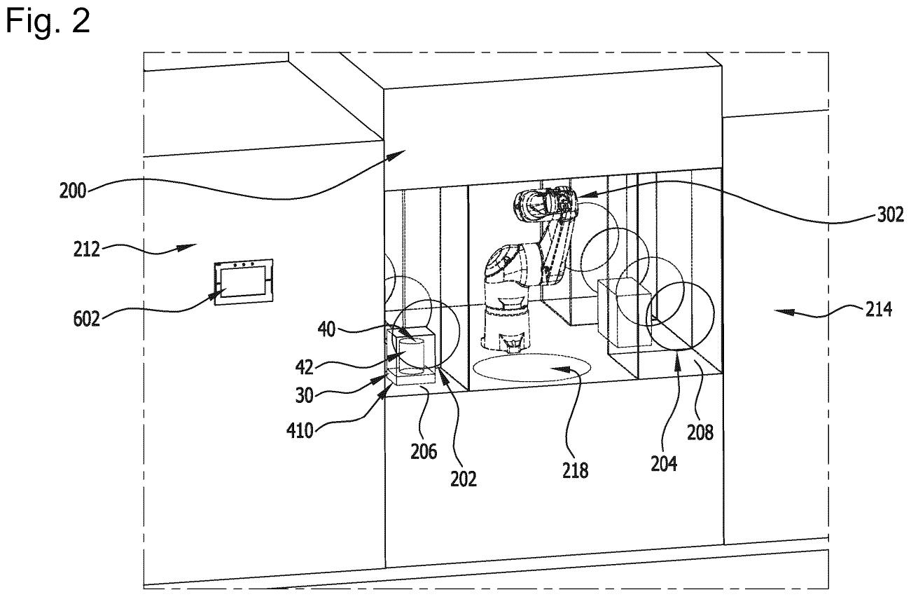

As is shown in FIGS. 1 and 2, the airlock device 200 comprises an entry airlock 202, via which an airlock space 206, in particular an entry airlock space 206, is accessible from the surroundings and which is configured to feed an object from the surroundings of the production system 10 into the clean room area 100.

As is furthermore shown in FIGS. 1 and 2, the airlock device 200 comprises an exit airlock 204, via which an airlock space 208, in particular an exit airlock space 208, is accessible from the surroundings and which is configured to remove an object from the clean room area 100.

The entry airlock 202 and the exit airlock 204 furthermore comprise means for in particular spatial isolation of the respective airlock spaces 206, 208 (not shown in the figures) associated with the entry airlock 202 and the exit airlock 204.

Isolation can optionally be carried out toward the surroundings of the production system 10 and/or toward a respective part of the airlock device 200 different from the airlock spaces 206, 208 in each case by means of the respective means for isolation from the entry airlock 202 and from the exit airlock 204.

Furthermore, the production system 10 comprises multiple maintenance airlocks 210, via which the clean room area 100 is accessible from the surroundings of the production system 10, for example for users, in particular for maintenance work inside the clean room area 100.

The production system 10 comprises a control unit, by means of which and/or via which the production system 10 can be operated, in particular regulated and/or controlled.

The control unit is/will be formed by all computing units of the processing units. In particular, the control unit is therefore formed by means of the respective computing units of the patient boxes 30, 50, the tool boxes 702, 704, the handling device 300, the storage transport device 406, the transport vehicles 410, etc.

In a certain sense, the control unit is a distributed control unit, which is based on a group, in particular a swarm, of individual computing units of the processing units of the production system 10.

The control unit can be connectable or connected, for example, to a control supplemental system 600 arranged in control cabinets, which can be designed and programmed in particular to regulate and/or control at least one component or a component part of the production system comprised by the production system, in particular in a supplementary manner. The control supplemental system 600 is configured, for example, in particular to carry out supplementary and/or subordinate and/or trivial processes, which the control unit does not execute and which are not security-relevant for the production system 10.

The control unit is designed and programmed to be able to communicate with one or more communication interfaces (not shown in the figures) and/or one or more human-machine interfaces, in short: HMI interfaces 602 of the production system 10 (an HMI interface 602 is shown by way of example in the figures).

The communication interfaces (not shown in the figures) can be used, for example, to ensure a data exchange with a higher-order guidance system and/or data management system, for example an associated cloud system, for example to enable data evaluation and/or data optimization via artificial intelligence and/or deep learning.

The control unit is furthermore designed and programmed to provide information to a user via the communication interfaces and/or via the HMI interfaces 602, for example interference information and/or information about a status of a manufacture of a product 20 and/or information about storage stocks of the storage device 400.

Furthermore, the manufacturing of the biological-pharmaceutical product 20 by means of the production system 10 can be carried out by means of the control unit.

Said product manufacturing can be carried out according to one or more production programs by means of the control unit.

In particular, the control unit is designed and programmed to associate a or the educt 40 with one of the multiple processing units 30, 50, 702, 704 on the basis of one of multiple production programs for manufacturing one of multiple products 20 that can be manufactured by means of the production system.

As shown in FIG. 1, the production system 10 furthermore comprises a first computer system 800, which is associated with the production system 10, for data exchange with the production system 10 and a second computer system 900, which is associated with the production system 10, for providing items of release information.

The first and the second computer system 800, 900 are in particular arranged spatially partitioned or separated with regard to a remainder of the production system and with regard to one another.

The first and the second computer system 800, 900 are each designed as servers. In particular, the first computer system 800 is designed as a heartbeat server 800 and the second computer system 900 is designed as a release server 900.

The heartbeat server 800 and the release server 900 are connectable and/or connected to the production system 10, in particular to the control unit, preferably to the computing units of the processing units, in particular via the control supplementary system 600, which can be configured or is present as a communication mediator. Additionally or alternatively, the heartbeat server 800 and the release server 900 can in particular be directly or indirectly connectable and/or connected to the production system 10, in particular to the control unit, preferably to the computing units of the processing units, as is generally known in the prior art, for example via an Internet-based and/or radio-based technology.

Each product 20 to be manufactured is associated with a sequence of treatment procedures, which can be carried out by means of processing units, such as tool units and/or tool boxes 702, 704, which are arranged inside the clean room area 100 and remain in the clean room area after the treatment procedures are carried out.

The treatment procedures required for the manufacturing of a specific product 20 and associated treatment time values are stored or taken into consideration as instructions in each production program.

In particular, a production program comprises multiple encrypted production program packets.

The processing units, thus in particular the patient boxes 30, 50 and/or the tool boxes 702, 704 are designed and programmed to save one or more cryptographic keys, by means of which the production program packets of the production program can each be decrypted, in particular locally on the respective processing unit.

The one or the multiple cryptographic keys are integrated inseparably in the processing units. For this purpose, each processing unit comprises a security module, in particular a trusted platform module.

Each production program packet contains at least one item of information which refers to another production program packet and comprises instructions for accommodating and/or treating an educt 40.

In other words, a production program packet comprises the instructions for one or more treatment procedures.

A defined order of the multiple production program packets forms the production program for manufacturing a product 20 that can be manufactured by means of the production system 10. This order represents the above-mentioned sequence of treatment procedures.

In particular, the complete order is only determinable from all references with respect to the other production program packets, since none of the production program packets enables a complete overview of the entire production program.

The production program packets comprise in particular a starting packet and one or more following packets. That is to say, a starting packet identifies a beginning of a production program.

The processing units, thus in particular the patient boxes 30, 50 and/or the tool boxes 702, 704, are designed and programmed to execute instructions of at least one production program packet, in particular to decrypt and then execute them.

All production program packets are each provided on one processing unit and/or are each transmittable to one processing unit, in particular by means of a computer system that is associated or can be associated with the production system 10, for example the release server 900.

The production system 10 is configured to be operable according to various statuses, which depend on an existence of a data connection to the heartbeat server 800. This is to be described in particular hereinafter.

As can be seen in particular in FIG. 1, the heartbeat server 800 is connectable or connected (cf. dashed line) to the production system 10.

The control unit is designed and programmed to be connectable or connected for data exchange to the above-described heartbeat server 800, to in particular continuously determine an existence of this data connection.

In particular, this heartbeat server 800 is used so that the production system 10 can only be operated over a specific time without data connection. In other words, the production system 10 can only produce if a connection to the heartbeat server 800 exists.

The production system and the processing units, i.e. in particular the patient boxes 30, 50 and/or the tool boxes 702, 704, are reversibly transferable depending on the data connection
into a normal status for unrestricted operation,
into a reserve status for chronologically and/or functionally restricted operation, and/or
into a pausing status for idled operation

The reserve status is a transition status between the normal status and the pausing status, via which the normal status passes into the pausing status, wherein in particular a transition from the pausing status to the normal status does not have to take place via the reserve status and preferably a transition from the pausing status to the normal status can take place directly.

The production system 10 for manufacturing a product 20 or in particular the patient boxes 30, 50 and/or the tool boxes 702, 704 for accommodating and/or treating an educt 40 are unrestrictedly operable only if the data connection exists. This is the normal status.

If the data connection does not exist, the production system 10 or in particular the patient boxes 30, 50 and/or the tool boxes 702, 704 for accommodating and/or treating an educt 40 are transferable into a reserve status, in which the manufacturing of a product 20 or in particular the accommodation and/or the treatment of an educt 40 is chronologically and/or functionally restricted.

The control unit and/or the computing units of the relevant processing units store an information stamp, in particular a timestamp, upon transfer into the reserve status and stop an unrestricted operation of the production system 10 or in particular the patient boxes 30, 50 and/or the tool boxes 702, 704 after a predefined period of time from this information stamp, in particular timestamp, and transfer the production system 10 or the relevant boxes into the pausing status.

Upon transfer into the reserve status, only a predefined part of a production program for manufacturing a product 20 is still executed. This is used, for example, to ensure production or treatment progress on an educt 40 and/or to bridge a possible short-term interruption of the data connection.

Upon transfer into the pausing status, the running production programs are modified and/or supplemented (in particular by means of the control unit) using one or more production program packets to place in storage the educts 40 and/or an at least partially treated educt 40 and/or a product 20 as long as the pausing status exists.

In other words, at least one production program is modified and/or supplemented using one or more production program packets, wherein the production program packets comprise instructions to place in storage.

Placement in storage takes place in particular in or on at least one of the storage spaces 402 of the storage device 400.

A modification and/or a supplementation can comprise, for example, targeted temperature control, for example freezing, of an educt 40 and/or an at least partially treated educt 40 and/or a product 20 in order to secure a production or treatment progress. This can take place at a storage space 402.

In a certain sense, the determination of an existence of the data connection can be viewed as a security mechanism of the production system 10.

The production system 10 is furthermore configured to comprise one or more further security mechanisms.

A further above-mentioned security mechanism is to be described in particular hereinafter:

In particular, the production system 10 is configured to determine an integrity of each processing unit, i.e. in particular the patient boxes 30, 50 and/or the tool boxes 702, 704.

With respect to the relevant processing units, this integrity determination, on the one hand, can be carried out from outside the relevant processing units and, on the other hand, can be carried out by the relevant processing units themselves. These two options can each be provided alone or in combination in the production system 10.

On the one hand, the control unit is designed and programmed to determine a or the integrity of each processing unit, i.e. in particular the patient boxes 30, 50 and/or the tool boxes 702, 704.

On the other hand, each computing unit is designed and programmed to determine a or the integrity of the respective processing unit, i.e. in particular patient box 30, 50 and/or tool box 702, 704.

This takes place before an educt 40 can be accommodated and/or treated by means of the processing unit or the relevant box 30, 50, 702, 704 according to one or more production program packets.

A goal of the integrity determination is in particular to being able to establish changes to the processing units, in particular the patient boxes 30, 50 and the tool boxes 702, 704. For example, it can therefore be detectable whether means of spying such as cameras and/or other comparable hardware have been attached by unauthorized parties in or on the processing units in order to be able to draw inferences about the manufacture of the product 20.

For this purpose, the computing unit of a processing unit or a relevant box 30, 50, 702, 704 is designed and programmed to provide a unique item of information related to this processing unit or box.

A further item of unique information related to this processing unit or box 30, 50, 702, 704 is detectable by means of the control unit. This relates to the integrity determination from outside the relevant processing unit or box 30, 50, 702, 704.

The integrity of this processing unit or box 30, 50, 702, 704 is determinable based on a comparison of these two items of unique information by means of the control unit.

If these two items of unique information correspond, the integrity is settable and/or confirmed. Setting and/or confirming of the integrity can take place in particular in a respective computing unit and/or the at least one control unit.

The unique item of information that can be provided on the part of the computing unit represents a setpoint status and/or a starting status of the processing unit or box 30, 50, 702, 704, on the basis of which a deviation with respect to the processing unit or box 30, 50, 702, 704 is determinable.

The unique item of information is stored on the above-described security module of the respective processing unit or box 30, 50, 702, 704 and can be provided for the integrity determination.

In particular, the item of unique information is to be understood as an item of digital fingerprint information, for example, an item of information which is unique and/or comprises a unique identifier.

A processing unit or box 30, 50, 702, 704 is designed and programmed to execute, thus to decrypt and then execute, instructions of a production program packet only if its integrity is set and/or confirmed.

In other words, treatment procedures are only carried out by means of the boxes 30, 50, 702, 704 if these boxes 30, 50, 702, 704 are intact in particular with respect to damage and/or manipulation.

Execution and/or decryption instructions are blocked if an integrity of the relevant boxes 30, 50, 702, 704 cannot be set and/or cannot be confirmed.

Furthermore, a processing unit or box 30, 50, 702, 704 is designed and programmed to provide a message, in particular an alarm message, for example via the HMI interface 602, if its integrity cannot be set and/or cannot be confirmed.

The production system comprises one or more sensor units for determining the integrity, by means of which the processing units or boxes 30, 50, 702, 704 are in particular visually detectable.

On the basis of this detection, a unique item of information with respect to the respective processing units or boxes 30, 50, 702, 704 can be provided by means of the control unit to determine the integrity.

The one or more sensor units comprise one or more camera units, by means of which characteristic landmarks of a face, in particular a surface, and/or a geometry of the processing units or boxes 30, 50, 702, 704 are visually detectable.

In other words, the unique information is based on characteristic landmarks of a face, in particular a surface, and/or a geometry of the processing units. The geometry can be a two-dimensional and/or a three-dimensional geometry.

It is to be understood that numerous further options are additionally or alternatively conceivable with respect to the unique items of information, for example, that:
the production system 10 comprises one or more actuator units, by means of which one or more processing units or boxes 30, 50, 702, 704 can be mechanically excited in particular to generate an in particular mechanical oscillation, which is detectable by means of the one or more sensor units and on the basis of which a unique item of information with respect to the respective processing units or boxes 30, 50, 702, 704 can be provided by means of the control unit to determine the integrity; wherein in particular:
   one of the one or more actuator units is configured to generate a mechanical oscillation and this actuator unit is arranged on or in one of the one or more processing units or boxes 30, 50, 702, 704 and/or can be engaged with one of the one or more processing units or boxes 30, 50, 702, 704 to generate the mechanical oscillation on the relevant processing unit or box 30, 50, 702, 704; wherein optionally:
      an actuator unit is designed as a motor, in particular a vibration motor, by means of which predeterminable frequency ranges can be generated and/or driven; and/or
      the oscillation comprises a frequency range of greater than or equal to 10 Hz and less than or equal to 100 Hz, and/or the oscillation comprises one or more in particular different frequency ranges, which are respectively greater than or equal to 10 Hz and less than or equal to 100 Hz, and/or the oscillation comprises one or more in particular different frequency ranges which follow one another in a specific order; and/or
   one of the one or more actuator units is configured to generate an acoustic oscillation, in particular a soundwave, and this actuator unit is arranged on or in one of the one or more processing units or boxes 30, 50, 702, 704 and/or can be engaged with one of the one or more processing units or boxes 30, 50, 702, 704 to generate the mechanical oscillation, in particular the soundwave, with respect to the relevant processing unit or boxes 30, 50, 702, 704; wherein optionally:
      an actuator unit is designed as a sound transducer, in particular a loudspeaker, by means of which predeterminable frequency ranges can be generated and/or driven; and/or
      the (acoustic) oscillation comprises a frequency range of greater than or equal to 30 Hz and less than or equal to 10 000 Hz, and/or the (acoustic) oscillation comprises one or more in particular different frequency ranges, which are respectively greater than or equal to 30

Hz and less than or equal to 10 000 Hz, and/or the (acoustic) oscillation comprises one or more in particular different frequency ranges which follow one another in a specific order.

It is to be understood that the preceding list of the means for integrity determination is by way of example and is not exhaustive.

It is also to be understood that any combination of the components described with respect to the integrity, in particular actuator units and/or sensor units, can be conceivable with respect to the respective processing units or boxes 30, 50, 702.

As already described, furthermore each computing unit is designed and programmed to determine a or the integrity of the respective processing unit, i.e. in particular patient box 30, 50 and/or tool box 702, 704.

This relates to the integrity determination by the relevant processing unit or box 30, 50, 702, 704 itself.

In other words, each processing unit or box 30, 50, 702, 704 can be designed as a self-checking processing unit, which is configured to determine its own integrity or in particular to be able to determine unauthorized changes to it itself.

The computing unit also provides a or the item of unique information related to this processing unit or box 30, 50, 702, 704 here, however, a or the further item of unique information related to this processing unit or box 30, 50, 702, 704 is detectable by means of the computing unit itself.

The integrity of the relevant processing unit or box 30, 50, 702, 704 is determinable based on a comparison of these two items of unique information by means of the respective computing unit.

A processing unit, in particular a patient box 30, 50 and/or a tool box 702, 704, can comprise one or more sensor units, by means of which a or the unique item of information and/or a or the further unique item of information are detectable via the relevant processing unit or box 30, 50, 702, 704 to determine the integrity.

A brightness value in the relevant processing unit or box 30, 50, 702, 704 is detectable by means of at least one sensor unit and the unique item of information and/or the further unique item of information about the relevant processing unit or box 30, 50, 702, 704 can be provided based on the detected brightness value to determine the integrity. For example, a detection of light can suggest a manipulation and/or an attack on the relevant processing unit or box 30, 50, 702, 704 and, if it is dark, the relevant processing unit or box 30, 50, 702, 704 is intact, thus a or the integrity is confirmed.

In addition, it can be provided that the relevant processing unit or box 30, 50, 702, 704 is at least partially visually detectable by means of at least one sensor unit and the unique item of information and/or the further unique item of information about the relevant processing unit or box 30, 50, 702, 704 can be provided based on the visual detection to determine the integrity, wherein the at least one sensor unit comprises a camera unit, by means of which characteristic landmarks and/or geometries of the relevant processing unit or box 30, 50, 702, 704 are visually detectable. The sensor unit is in particular configured inside and/or on an exterior of the processing unit or box 30, 50, 702, 704.

It is to be understood that numerous further options are additionally or alternatively conceivable with respect to the unique items of information, for example, that:
a processing unit or box 30, 50, 702, 704 comprises one or more actuator units, by means of which the relevant processing unit can be mechanically excited in particular to generate an in particular mechanical oscillation, which is detectable by means of the one or more sensor units and on the basis of which the unique item of information and/or the further unique item of information with respect to the relevant processing unit or box 30, 50, 702, 704 can be provided to determine the integrity, wherein optionally:
   the oscillation comprises a frequency range of greater than or equal to 10 Hz and less than or equal to 100 Hz, and/or the oscillation comprises one or more in particular different frequency ranges, which are respectively greater than or equal to 10 Hz and less than or equal to 100 Hz, and/or the oscillation comprises one or more in particular different frequency ranges, which follow one another in a specific order, wherein in particular at least one of the one or more actuator units is designed as a vibration motor, which is fixed in and/or on the relevant processing unit to generate the mechanical oscillation; and/or
at least one of the one or more actuator units is configured to generate an acoustic oscillation, in particular a soundwave, and is fixed in and/or on the relevant processing unit or box 30, 50, 702, 704 to generate the mechanical oscillation, in particular the soundwave, with respect to the relevant processing unit or box 30, 50, 702, 704; wherein optionally:
   the oscillation comprises a frequency range of greater than or equal to 30 Hz and less than or equal to 10 000 Hz, and/or the oscillation comprises one or more in particular different frequency ranges, which are respectively greater than or equal to 30 Hz and less than or equal to 10 000 Hz, and/or the oscillation comprises one or more in particular different frequency ranges which follow one another in a specific order; and/or
a processing unit or box 30, 50, 702, 704 can comprise one or more electrically conductive line elements, which are arranged in and/or on the processing unit or box 30, 50, 702, 704 and/or are arranged inside a lateral surface wall of the processing unit or box 30, 50, 702, 704 and which form an in particular independent electric circuit, wherein it is detectable by means of one or more sensor units whether the electric circuit is changed with respect to its original status and/or is interrupted, and wherein the item of unique information and/or the further item of unique information about the relevant processing unit or box 30, 50, 702, 704 can be provided to determine the integrity on the basis of the circuit detection; and/or
at least one sensor unit can be designed as an inertial measuring unit, by means of which at least one acceleration value, in particular an acceleration vector, and/or at least one rotation rate value, in particular a rotation rate vector, is detectable with respect to the relevant processing unit or box 30, 50, 702, 704 and the unique item of information and/or the further unique item of information about the relevant processing unit or box 30, 50, 702, 704 can be provided based on the detectable acceleration and/or rotation rate values to determine the integrity; and/or a temperature value in and/or on the relevant processing unit is detectable by means of at least one sensor unit and the unique item of information and/or the further unique item of information about the relevant processing unit can be provided based on the detectable temperature value to determine the integrity.

It is to be understood that the preceding list of the means for integrity determination is by way of example and is not exhaustive.

It is also to be understood that any combination of the components described with respect to the integrity, in particular actuator units and/or sensor units, can be conceivable with respect to the respective processing units or boxes 30, 50, 702.

A further above-mentioned security mechanism is to be described in particular hereinafter:

At least a part of a or the production program is in particular used exclusively to emulate and/or suggest one or more production program procedures, in particular one or more treatment procedures.

In this way, it can be achievable that real or actual production steps or production program procedures are or become concealed, so that in particular potential attackers would have to employ further examinations to be able to reconstruct a manufacture of a product 20.

A or the production program comprises one or more encrypted dummy production program packets for emulation and/or suggestion.

Each dummy production program packet contains at least one item of information which refers to a (correct) production program packet or another dummy production program packet and which comprises dummy instructions for emulating and/or suggesting an accommodation and/or a treatment of an educt 40. For example, a dummy production program packet, in particular a dummy instruction, can in particular be empty of content for production technology.

The production system 10 comprises a random module, which is executable by means of the computing units and by means of which one or more dummy production program packets are integratable in a production program in a random manner.

A processing unit or box 30, 50, 702, 704 is designed and programmed to execute, in particular to decrypt and then execute, any instructions, in particular actual/correct instructions and/or dummy instructions.

Furthermore, a processing unit or box 30, 50, 702, 704 is designed and programmed to accommodate and/or treat a dummy educt, in order to emulate and/or suggest an accommodation and/or a treatment of an actual educt 40 using the dummy educt.

For example, a dummy educt can be an empty container 42 intended for an actual educt 40, which cannot be seen into from the outside, and/or can comprise a material similar to an actual educt 40.

In addition, the production system 10 comprises one or more concealment chambers for accommodating one or more processing units, wherein an interior of each concealment chamber is optically separated and/or at least substantially cannot be seen into from outside the concealment chamber.

A processing unit or box 30, 50, 702, 704 can be caused to be accommodated in the interior of a relevant concealment chamber by means of the part of the production program for emulation and/or suggestion.

For example, the concealment chamber can be formed by means of one or more storage spaces 402. Additionally or alternatively, a concealment chamber can be arranged as an independent apparatus in the clean room area 100.

With reference to FIGS. 1 and 9, the concealment chamber is accessible via the storage rack airlock 412 for the processing units designed as the box 30, 50, 702, 704.

As can be seen in FIGS. 1 and 9, the concealment chamber can substantially or completely not be seen into by persons who stand outside or also inside the production system 10, in particular the clean room area 100.

For example, it can also be provided that an optical separation can relate to a wavelength range visually perceptible by humans and/or to a wavelength range not visually perceptible by humans, for example an infrared range.

The concealment chamber comprises one or more changing units (not shown in the figures), by means of which objects which are accommodated in and/or by processing units, in particular patient boxes 30, 50 and/or tool boxes 702, 704, can be rearranged between the relevant processing units which are located inside the concealment chamber. Corresponding instructions are comprised by dummy production program packets.

In other words, objects can be exchanged between the relevant processing units like a shell game by means of the concealment room or its changing units.

The concealment chamber furthermore comprises one or more sensor units for monitoring the optical separation of the interior from the exterior of the concealment chamber, wherein in particular a brightness value inside the concealment chamber is detectable by means of at least one sensor unit of the concealment chamber.

A further above-mentioned security mechanism is to be described in particular hereinafter:

This security mechanism is based in particular on production program packets only being executable by a relevant processing unit, in particular box 30, 50, 702, 704, if the relevant processing unit or box 30, 50, 702, 704 has had a separate release input or release authorization provided by the above-described release server 900.

The release server 900 is designed and programmed to at least partially store and operate a database.

The control unit is again designed and programmed to exchange information with this database.

As can be seen in particular in FIG. 1, the release server 900 is connectable or connected (cf. dashed line) to the production system 10.

The database contains multiple unique items of release information to authorize an accommodation and/or a treatment of an educt 40 or a treatment procedure by means of the processing units, in particular the boxes 30, 50, 702, 704.

The database is changeable by a retrieval of at least one of the items of release information for one of the multiple processing units or boxes 30, 50, 702, 704, so that this release information can subsequently no longer be provided in the original form for retrieval.

In particular, it is stored in the database that the release information was already transmitted and/or used, so that it can no longer be provided in the original form for retrieval.

In particular, the unique release information can be understood as an item of information which is unique and/or comprises a unique identifier.

The database is configured and programmed to receive and store one or more new items of release information exclusively from outside the production system.

In other words, the database can be supplied with one or more new items of release information exclusively from outside the production system.

For example, one or more new items of release information can be transmittable to the database directly on the part of a database access authorized party 1000, in particular a rights holder of a formula represented by a production program, in particular via a separate computer system 1000, for example via a cloud and/or a server. This computer system 1000 can optionally be part of the production system 10.

It is conceivable that the database is configured and programmed to be storable and/or operable exclusively on the release server 900, or to be able to be synchronized with a parallel database, which is storable and/or operable by means of the control unit and/or the separate computer system 1000.

Each computing unit of each processing unit, in particular each box 30, 50, 702, 704, is configured and programmed to receive and store one or more items of release information.

In particular, each item of release information received by a computing unit can be compared with each item of release information already stored on a computing unit to avoid a double use of the received release information in the production system 10.

In other words, all computing units are networked with one another, in particular for said comparison of the received items of release information to avoid a double use.

For this purpose, each computing unit is designed and programmed to store and operate an encrypted decentralized database, preferably an in particular encrypted distributed ledger, in order to store the one or more receivable or received items of release information.

A manipulation-secured technical solution can be provided by the use of the decentralized database in order to avoid a double use of the received release information in the production system 10. The decentralized database will be described in more detail in the following description.

Each production program packet can be uniquely associated with one of the items of release information to authorize an in particular one-time execution of this production program packet by means of a processing unit, in particular a box 30, 50, 702, 704. It can be ensured in this way that the execution of production program packets is not possible beyond a released limit or a released execution number.

In particular, each production program can only be able to be initialized and/or started by means of the production system if all production program packets of the production program and all items of release information associated with the production program packets can be provided or are provided on the relevant processing units or boxes 30, 50, 702, 704.

In particular, the items of release information are receivable and storable for this purpose by means of the computing units.

This can take place in particular for a time-delayed authorization and execution of an accommodation and/or treatment of an educt 40 and/or for an immediate authorization and execution of an accommodation and/or a treatment of an educt 40.

Each item of release information in the database can be encrypted or is encrypted and can be decrypted by means of at least one cryptographic key, which can be provided and/or stored by means of a relevant computing unit, in particular its security module, and/or is stored therein.

It is to be understood that the statements with respect to the items of release information likewise relate to the above-described dummy production program packets of the production program.

In other words, the dummy production program packets are also releasable by means of one or more items of release information of the release server 900.

It is conceivable that one or more items of release information are retrievable from the database as bundled release information for authorizing an overall process for manufacturing a product 20, in particular by means of one or more processing units. Additionally or alternatively, it can be provided that an item of release information is individually retrievable from the database for each individual production program packet, in particular by means of a processing unit.

The database is furthermore configured and programmed to associate one or more, in particular all items of release information with a respective item of confirmation information, which is storable in the database for each item of release information, in particular by a database access authorized party 1000, wherein each item of release information can only be provided if an associated item of confirmation information is stored.

In other words, the database access authorized party 1000 can confirm a use of individual or multiple items of release information on the release server 900. For example, in this way a separate, in particular automated invoicing system of products to be manufactured can be implementable and/or an authority over a manufacture of a product 20 can remain with a rights holder.

It is conceivable that each item of release information comprises an information stamp, in particular a number information stamp, by means of which an in particular maximum repeatability of an accommodation and/or a treatment of an educt 40 or a treatment procedure is settable, which can be authorized by means of the relevant release information.

Moreover, it is to be understood that the possibility of authorizing treatment procedures by means of the items of release information depends on the existence of the data connection of the production system 10 to the heartbeat server 800, thus on the normal status, reserve status, and/or pausing status.

As already described, an execution of treatment procedures, in particular an execution of production program packets and/or a use of items of release information can be monitored on the part of a processing unit, in particular a box 30, 50, 702, 704, by means of a decentralized database, which is operated by the respective computing unit of a processing unit or box 30, 50, 702, 704.

Each computing unit is designed and programmed to store and operate an encrypted decentralized database, preferably an encrypted distributed ledger.

Each decentralized database is a redundant copy of each of the other decentralized databases, which are stored on the computing units.

Each computing unit is designed and programmed to validate an operation of the respective decentralized databases on each of the other computing units and to update its own decentralized database in response to a validation.

Each decentralized database is furthermore designed and programmed to comprise one or more, in particular unique items of information about an accommodation and/or a treatment of an educt 40 by each processing unit, in particular each box 30, 50, 702, 704.

In particular, the unique information is to be understood as an item of information which is unique and/or comprises a unique identifier. The unique information comprises in particular one or more items of release information, one or more items of information about executed and/or non-executed production program packets, etc.

Each decentralized database can be able to be encrypted or can be encrypted by means of a cryptographic key, which can only be associated with and/or provided to the processing unit on which the decentralized database is storable or stored. The cryptographic key is in particular stored in the above-described security module.

Each computing unit is configured and programmed to store each execution of a production program packet in the decentralized database depending on the validation by the other computing units.

Each computing unit is furthermore configured and programmed to execute a production program packet only if this production program packet is not stored as already executed in the decentralized database.

Each computing unit is furthermore configured and programmed to store the items of release information in its own decentralized database and to compare each received item of release information with each item of release information already stored in the decentralized database to avoid a double use of the received release information in the production system, depending on the validation by the other computing units.

The instructions of a production program packet are only executed by a processing unit or box 30, 50, 702, 704 if an item of release information associated with this production program packet is provided to the computing unit and in particular this production program packet and this release information is stored in the decentralized database as not yet already executed.

In other words, a permission to execute a production program packet can be able to be granted by means of the described release information mechanism, which permission is storable in particular in the decentralized database, wherein it is checkable in particular upon each execution of a production program packet whether the number of the previous executions is less than the permitted number of executions of the production program packet, and wherein it is storable in particular in the decentralized database that a production program packet will be or is executed or can be executed, by which said number of available executions is reducible by 1 (one) upon actual execution. It can be ensured in this way that the execution of production program packets is not possible beyond the released limit.

It is conceivable that the production system 10 comprises one or more further computer systems and/or is connectable or connected to such further computer systems, wherein each of the further computer systems 10 is arranged in a different location with respect to a remainder of the production system, wherein the decentralized database is stored and operated on each of the further computer systems, and wherein each of the further computer systems is designed and programmed to validate an operation of the respective decentralized databases on each of the other computer systems and/or computing units and to update its own decentralized database in response to a validation. Such a computer system can in particular be formed by the heartbeat server 800 and/or the release server 900 and/or the attached computer system 1000.

In particular for the purpose of data evaluation and/or data optimization and/or deep learning of the production system 10, the production system 10 is configured to carry out a simulation of its own operation and/or in particular of treatment procedures.

In this way, for example, routes of the boxes 30, 50, 702, 704 and/or the transport vehicles 410 and/or the multiaxis robot arm 302 can be optimized or a corresponding route optimization is possible.

Furthermore, a prediction of the production sequence can be checked and/or updated, in particular to confirm or correct timeslots for resources for finishing the product 20.

In addition, a job planning of one or more products 20 and/or of production lots can be carried out, in particular in a chronological aspect and/or with regard to resources located inside the production system 10, for example, boxes 30, 50, 702, 704 and/or tools and/or consumable materials, and/or a current utilization of the production system 10.

For this purpose, the production system 10 comprises a simulation device, which is designed and programmed to simulate the operation of the production system 10 and/or in particular the manufacturing of a product 20 based on at least one production program and to provide a simulation result corresponding thereto.

Like the control unit, the simulation device is formed by all computing units of the respective processing units. It is conceivable that the control unit functions as the simulation device and/or executes or performs its function in the present exemplary embodiment.

The simulation device is designed and programmed to detect system parameters of the production system 10 during a manufacture of a product 20 and/or to detect accommodation procedure measured values and/or treatment procedure measured values of the processing units, in particular operating parameters of the processing units, and store them as simulation information and save them to carry out a simulation.

In other words, the simulation device is designed and programmed to detect:
process-specific parameters and/or measured values, for example an order of treatment procedures or processing steps, and/or
system-specific parameters and/or measured values, for example of processing units, in particular of patient boxes 30, 50 and/or tool boxes 702, 704, and/or location parameters of the production system 10 such as exterior temperature, ambient humidity, air pressure, and/or
patient-specific parameters and/or measured values, for example a medical treatment history, and/or
tool-specific parameters and/or measured values, for example, of tool units, in particular tool boxes 702, 704, and/or
material-specific parameters and/or measured values, for example of consumable materials, and to store them as simulation information and save them to carry out a simulation.

As described above, the production system 10 comprises one or more sensor units. These are at least partially associated with the processing units and/or form a part of the processing units.

The simulation device is designed and programmed to carry out the simulation on the basis of items of simulation information detectable or detected by means of these sensor units.

In particular, the sensor units can be designed and/or configured as described in the application DE 10 2021 207 738.2, to which reference is hereby made.

In particular, the part of a production program which is to be executed by means of the associated processing units can be simulated by means of the computing units forming the simulation device.

In other words, each processing unit, in particular each box 30, 50, 702, 704, is designed and programmed to simulate at least the part of a production program which the relevant processing unit or box 30, 50, 702, 704 has to and/or would execute to actually manufacture a product 20.

In still other words, each processing unit, in particular each box 30, 50, 702, 704, is designed and programmed to simulate at least one production program packet which the relevant processing unit or box 30, 50, 702, 704 has to and/or would execute to accommodate and/or treat an educt 40 during an actual manufacture of a product 20.

It is also provided in the simulation that the respective production program packets to be simulated are to be decrypted by the respective computing units, which was already described in conjunction with the (actual) execution of the production program packets.

Furthermore, the simulation likewise requires release by means of the release server 900, which was already described in conjunction with the (actual) execution of the production program packets.

That is to say, an execution of a simulation can be released by means of an item of release information receivable by the processing unit.

That is to say, each item of release information is designed to authorize either an actual execution of a production program packet or only its simulation.

Each item of release information comprises an information stamp, in particular a mode information stamp, for this purpose, by means of which the respective item of release information can be associated with a production mode of the production system 10 for manufacturing a product 20 or a simulation mode of the production system 10 for simulating the operation of the production system 10 and/or the manufacturing of a product 20, in particular one or more production program packets or treatment procedures.

In other words, depending on the information stamp of the release information, a production program packet can be executable by means of a processing unit, in particular the boxes 30, 50, 702, 704, for actually accommodating and/or treating an educt 40 or only for its simulation with respect to the relevant processing unit, in particular box 30, 50, 702, 704.

As already described above, a or the simulation can also at least partially or completely require release by means of one or more items of release information. A release of a simulation can essentially take place analogously to a release of an actual manufacture of a product 20. This will be described in more detail hereinafter in reference to a simulation.

The database of the release server 900 furthermore contains items of release information to authorize a simulation, wherein the database is changeable by a retrieval of at least one of the items of release information for a simulation so that this release information can no longer be provided in the original form for retrieval.

The database is furthermore configured and programmed to receive and store one or more new items of release information to authorize a simulation exclusively from outside the production system 10. For example, the new items of release information for a simulation can also be transmittable to the database on the part of the database access authorized party 1000.

Each production program packet can be uniquely associated with at least one of the items of release information to authorize an in particular one-time simulation.

In particular, a simulation can only be able to be initialized and/or started by means of the simulation device if all production program packets of the production program and/or all items of release information associated with the production program packets can be provided.

One or more items of release information are receivable and storable by means of a processing unit, in particular box 30, 50, 702, 704, for a time-delayed authorization and execution of a simulation of an accommodation and/or a treatment of an educt 40 and/or are receivable in real time for an immediate authorization and execution of a simulation of an accommodation and/or a treatment of an educt 40.

It is to be understood that a simulation by means of the simulation device also relates to dummy production program packets.

A simulation is thus in particular only able to be initialized and/or started if all production program packets including the dummy production program packets of the production program and/or all items of release information associated with the production program packets including the dummy production program packets can be provided or are provided.

Furthermore, it can be provided that the production system 10 is switchable to another status depending on the existence of a data connection the heartbeat server 800, which was already described. It can be provided in this case that a simulation can be carried out, or not, depending on the respective status.

As described above, the production system 10 comprises multiple, in particular a plurality of, sensor units.

In particular, the sensor units can be designed and/or configured as described in the application DE 10 2021 207 738.2, to which reference is hereby made.

In particular, the production system 10 comprises one or more sensor units for determining one or more values of one or more parameters of one or more educts 40 and/or one or more patient boxes 30, 50, and/or one or more tool boxes 702, 704, and/or one or more tools or tool units, and/or one or more consumable materials.

The sensor units are used in particular to determine a treatment progress during the treatment of an educt 40 or between two treatment procedures.

The sensor units are used in particular to control and/or regulate one or more tool units to carry out one or more treatment procedures depending on the one or more determined values, wherein one or more treatment parameters are varied or are changeable in particular depending on a determined treatment progress.

The sensor units are used in particular to monitor the storage device 400, for example, of storage statuses and/or storage amounts at the storage spaces 402.

Furthermore, as already described, at least a part of the sensor units of the production system 10 is arranged in and/or comprised by the processing units, in particular the patient boxes 30, 50 and/or the tool boxes 702, 704.

In this case, each sensor unit or at least each sensor unit of a processing unit, in particular the patient boxes 30, 50 and/or the tool boxes 702, 704, is configured to detect at least one measured value with respect to the accommodation and/or the treatment of the educt 40 and/or with respect to the processing unit itself and to provide the measured value to the respective computing unit for transmission to one or more reception-authorized units in encrypted and/or abstracted form.

In this way, measured values and/or information can be made unidentifiable on the processing unit side, so that an information outflow, for example by means of a manipulative tap, to an unauthorized party is avoidable.

In other words, at least one measured value, which is detectable by means of the one or multiple sensor units, can first be made/become unidentifiable before it is transmittable to one or more reception-authorized units.

In the present exemplary embodiment, it is provided that the one or more reception-authorized units is one or more other processing units and/or the control unit of the production system 10.

An item of information which represents the at least one detectable measured value is modifiable by means of the computing unit and the one or more reception-authorized units are designed and programmed to interpret the modified information.

It is provided that the information is modifiable in an encrypted manner and/or in an abstracting manner and the information can be decrypted and/or made specific by means of the one or more reception-authorized units.

In other words, a modification comprises an encryption and/or an abstraction.

As already described, an encryption also refers here to a public-key encryption.

An abstraction can comprise, as a simple, nonrestrictive example, in particular an omission of in particular physical units. Only the one or more reception-authorized units can interpret the solely numeric value and/or assign the correct, in particular physical unit thereto again.

An information depth or an information content of the at least one measured value can therefore be able to be retained. In other words, therefore no information loss can occur.

Each computing unit is configured and programmed to provide the information only or exclusively as modified information for transmission to the one or more reception-authorized units.

The transmitted items of information are stored in the one or more reception-authorized units, in particular for process optimization of the production system 10. In particular, these items of information are used as or form the described simulation information.

Each computing unit furthermore comprises a communication module, in particular a Bluetooth module, preferably a Bluetooth low-energy module, and/or a Wi-Fi module.

Each computing unit is furthermore configured and programmed to form a network with each other computing unit for information transmission by means of the at least one communication module.

It is to be understood that the communication module can be alternatively designed. For example, the communication module can be based on: a ZigBee technology, a 6LoWPAN technology, a LoRaWAN technology, a NB-IoT technology, a LTE technology, a 5G technology, a Wi-Sun technology, etc.

Reference is made hereinafter in particular to FIGS. 2 to 11;

FIG. 2 shows an enlarged view in particular of the airlock device 200 of FIG. 1.

It is in particular recognizable here that the handling device 300 comprises a multiaccess robot arm 302, which is arranged adjacent to the airlock device 200.

Objects are movable in the airlock device 200 by means of one of the freely moving transport vehicles 410, in particular one or more objects are transportable from an airlock space, in particular an entry airlock space 206, to a further airlock space, in particular a transfer airlock space 216, and/or from an airlock space, in particular a transfer airlock space, to a further airlock space, in particular an exit airlock space 208.

Furthermore, with reference to FIG. 2, a patient box 30, in which an educt 40 arranged in an educt container 42 is/will be accommodated and held, is arranged on a freely moving transport vehicle 410 at the entry airlock space 206.

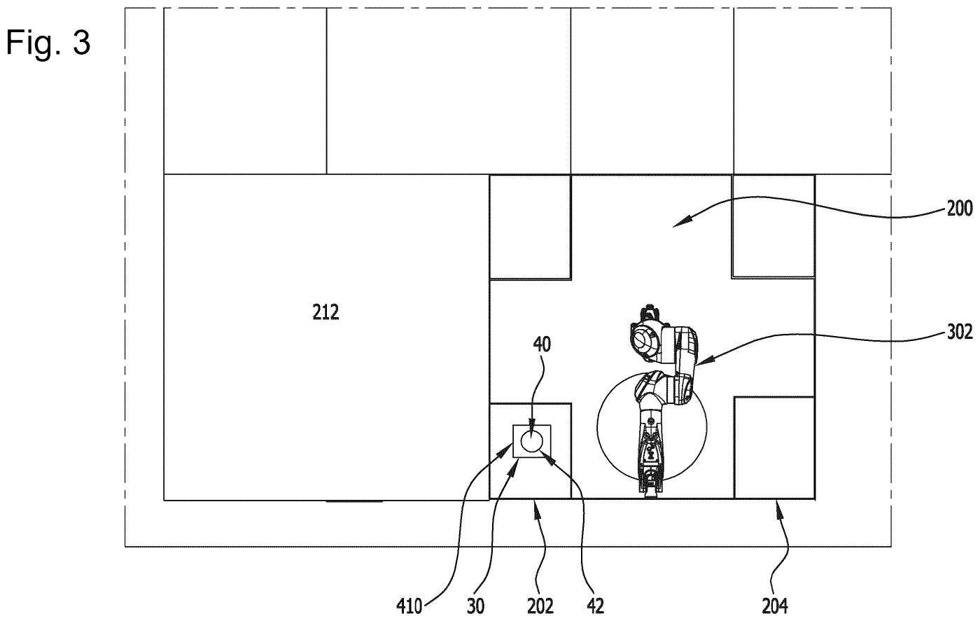

The patient box 30, 50 arranged on the transport vehicle 410, as shown in FIGS. 2 and 3, was fed by means of the transport vehicle 410 to the entry airlock space 206 to accommodate the educt container 42 in a procedure not shown in the figures.

In this case, a production program of the production system 10 was initially selected by a user, for example via the HMI interface 602, in order to manufacture the desired product 20 from the educt 40.

As soon as all items of release information from the release server 900 and all production program packets of the selected production program are present, the production program can be started.

It is to be understood that before each execution of a production program packet, the integrity of the relevant processing unit is determined and the production program packet is only executed if the integrity exists.

The control unit carries out an assignment of the educt 40 to be processed to a patient box 30, 50 to which the starting packet of the selected production program is assigned and/or on which the starting packet of the selected production program is stored.

After this assignment, the patient box 30, 50 is provided at the entry airlock space 206 on the transport vehicle 410. This procedure can also be able to be carried out based on a following packet of the starting packet, wherein in this case the transport vehicle 410 is the executing processing unit of said following packet.

As already explained, each procedure, in particular handling procedure, and/or each production program packet, in particular starting packet and following packets, requires an item of release information.

Furthermore, it is to be understood that the respective decryptions on the processing units respectively take place by means of the keys stored therein.

In other words, for each procedure, in particular treatment procedure, a production program packet, an item of release information, and one or more cryptographic keys come together in a relevant processing unit.

The user thereupon places the educt container 42 having the educt 40 arranged therein in the patient box 30, 50 provided at the entry airlock space 206, which was assigned to the accommodation of the educt 40 on the basis of the production program by means of the control unit.

It is conceivable that a patient box 30, 50 placed in storage at a storage space 402 of one of the storage racks 404 was removed from the storage space 402 by means of the transport vehicle 410 of the storage transport device 406 and was transported to the airlock device 200. As soon as the patient box 30, 50 is provided at the entry airlock space 206 to accommodate the educt 40, the educt container 42 having an educt 40 arranged therein is manually transferred by the user to the patient box 30, 50 arranged on the transport vehicle 410 and fixed thereon by means of a means for fixing.

In other words, a patient box 30, 50 has a means for fixing (not shown in the figures), in particular a means for formfitting and/or fiction-locked and/or magnetic fixing, for an accommodation of educts 40 and/or tools and/or consumable materials. This also applies for the tool boxes 702, 704.

The educt container 42 generally furthermore comprises a package for maintaining an aseptic status of the educt 40.

The entry airlock 202 is now transferable into a closed status or is now closed. This procedure can also be able to be carried out based on a subsequent following packet of the preceding following packet, wherein in this case the airlock device 200, in particular the entry airlock 202, is the executing processing unit of said following packet.

The cleaning and sterilization procedure is now carried out on the packaged educt container 42 at the entry airlock space 206 by means of gassing and/or irradiation by means of a correspondingly configured gassing source and/or irradiation source (not shown in the figures) at the entry airlock space 206. This procedure can also be able to be carried out based on a subsequent following packet of the preceding following packet.

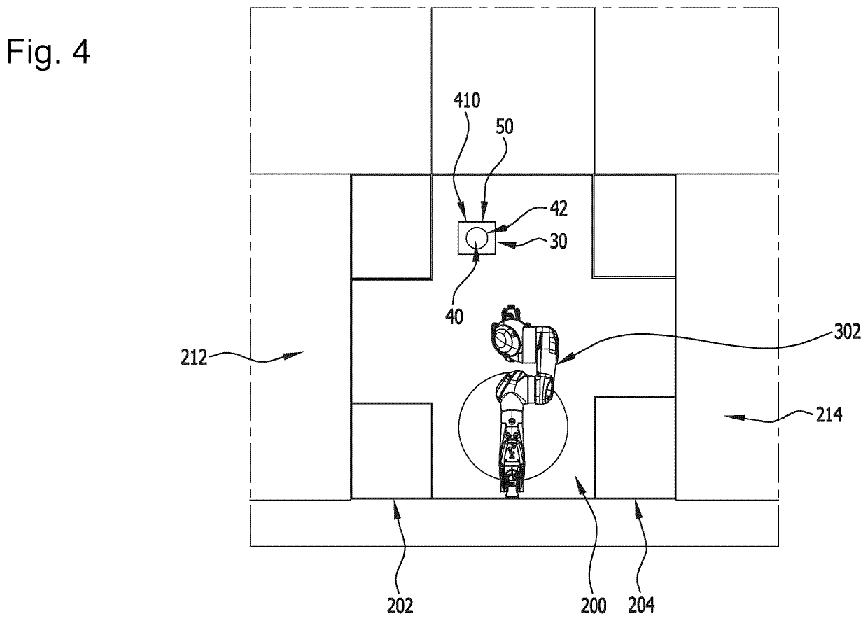

As schematically shown in FIG. 4, the packaged educt container 42 is subsequently moved on the workpiece carrier 30 by means of the transport vehicle 410 into a range of the multiaxis robot arm 302. This procedure can also be able to be carried out based on a subsequent following packet of the preceding following packet.

The multiaxis robot arm 302 takes the packaged educt container 42 out of the patient box 30, 50 and transfers the educt container 42 to the unpacking device 212, in which the packaging of the educt container 42 is removable/is removed by means of one or more unpacking units of the unpacking device 212. This procedure can also be able to be carried out based on a subsequent following packet of the preceding following packet.

After the transfer of the packaged educt container 42 to the unpacking device 212, the patient box 30, 50 is moved back again by means of the transport vehicle 410 to the entry airlock space 206 for a renewed accommodation of an object, so that essentially a status as shown in FIG. 3 can exist. This procedure can also be able to be carried out based on a subsequent following packet of the preceding following packet.

After the unpacking by means of the unpacking device 212, the unpacked educt container 42 is transferred, in another, in particular sterile patient box 30, 50, at the transfer airlock space 216 onto another sterile transport vehicle 410 (cf. FIG. 4) by means of the multiaxis robot arm 302. This procedure can also be able to be carried out based on a subsequent following packet of the preceding following packet.

The other patient box 30, 50 was previously removed from a storage space 402 of the storage racks 404 and was transported by means of the other transport vehicle 410 to the transfer airlock space 216. This procedure can also be able to be carried out based on a subsequent following packet of the preceding following packet.

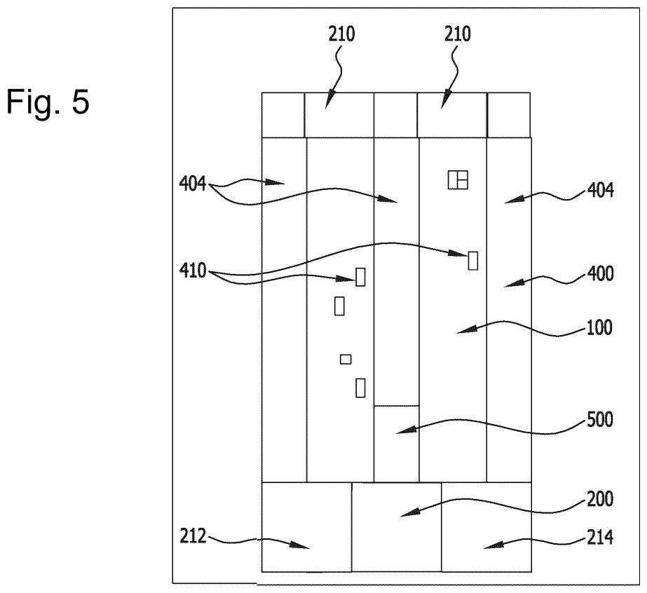

With reference to FIGS. 4 and 5, the receptacle box 30, 50 having the educt container 42 arranged therein is able to be moved and placed in storage or is moved and placed in storage into a storage space 402 in a storage rack 404 by means of the transport vehicle 410. This procedure can also be able to be carried out based on a subsequent following packet of the preceding following packet.

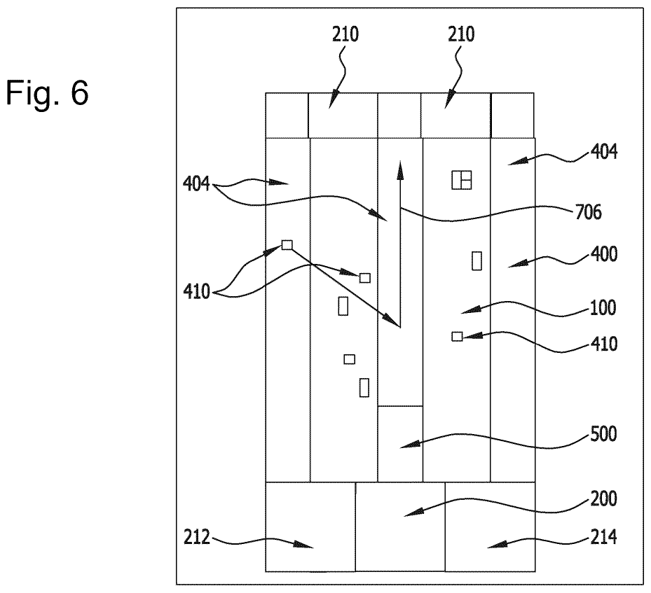

As shown by way of example in FIG. 6 by means of the arrows (tool path 706), for example, a tool box 702 for carrying out one or more treatment procedures is removed from the left storage rack 404 by means of a transport vehicle 410 and fed to the patient box 50 having the educt container 42, which is stored in the middle storage rack 404, in order to carry out the one or the multiple treatment procedures on the educt 40 in the educt container 42 by means of the tool box 702 or the tool units arranged and/or configured therein. This procedure can also be able to be carried out based on a subsequent following packet of the preceding following packet.

Said tool units are part of a tool system of the production system 10, by means of which treatment procedures on educts 40 and/or maintenance procedures can be carried out inside the clean room area 100.

The tool system comprises multiple in particular fully automatic tool units for carrying out different treatment and/or maintenance procedures.

The tool system comprises one or more tool units designed as feed units for feeding consumable materials to the educts 40 or the educt 40.

The tool system comprises one or more tool units designed as collecting units for collecting a sample from a or the educt 40.

One or more of the tool units of the tool system each comprises one or more multiuse components and/or one or more single-use components.

After carrying out the one or the multiple treatment procedures on the educt 40 has been completed, the previously delivered and now used tool box 702 is discharged again by means of the transport vehicle 410 from the patient box 30, 50, which contains the educt 40, in particular removed from or out of the patient box 30, 50 and transported away. These procedures can be able to be carried out based on multiple subsequent following packets.

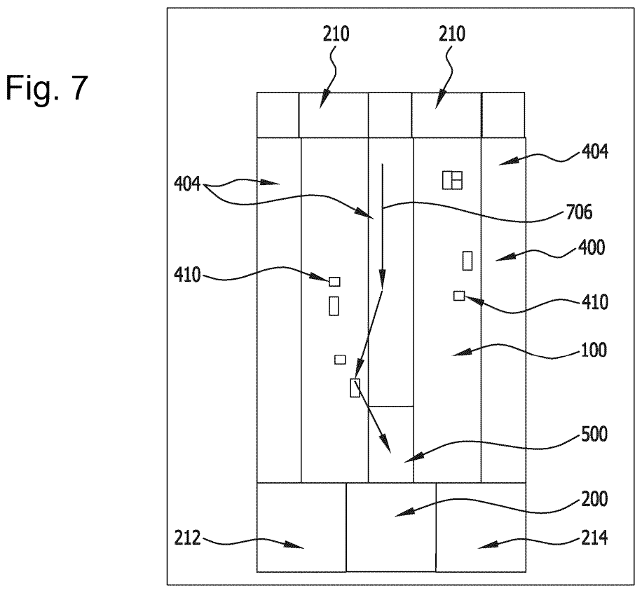

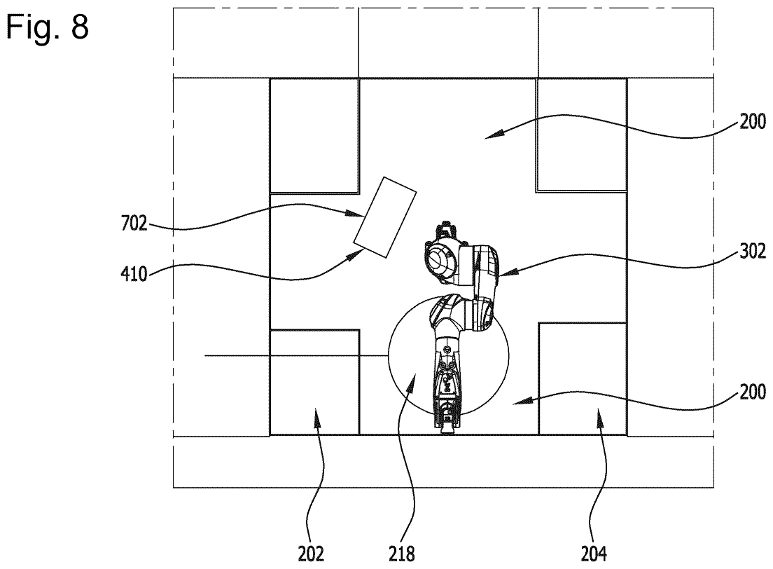

As shown by means of the arrows in FIG. 7, the transport vehicle 410 transports the used tool box 702 to the handling device 300, in order to remove single-use components configured in the tool box 702 there from the tool box 702 by means of the handling device 300 (cf. FIG. 8).

With reference to FIGS. 8 and 9, the used tool box 702 is moved back again, in particular by means of the handling device 300 and/or the transport vehicle 410 and subsequently fed to the cleaning device 500 of the production system 10 by means of the transport vehicle 410, by means of which the used tool box 702 is cleanable and sterilizable or will be cleaned and sterilized. This applies accordingly to tool units for multiple use which are arranged and/or configured in the tool box 702. This procedure can also be able to be carried out based on a subsequent following packet of the preceding following packet.

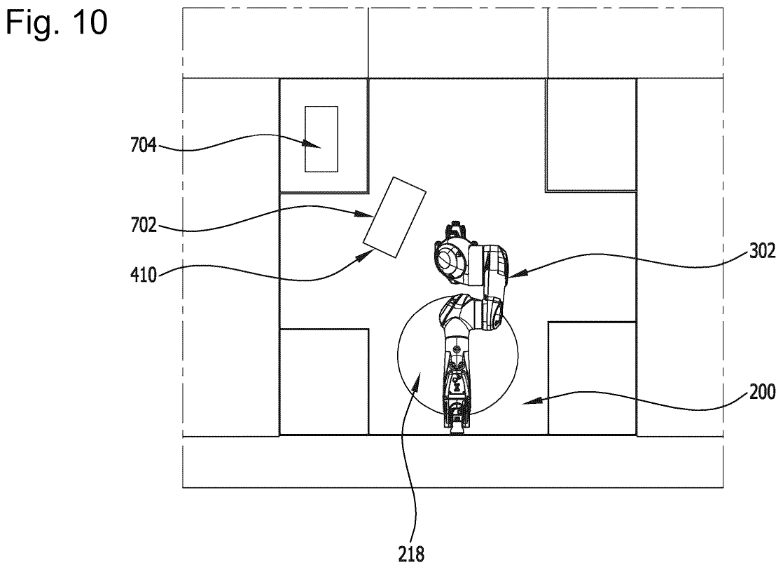

As schematically shown in FIG. 10, the tool box 702 cleaned and sterilized by means of the cleaning device 500 is fed to the handling device 300 again by means of the transport vehicle 410, in order to be equipped again with consumable material, in particular one or more new sterile single-use components from the storage racks 404, by means of the handling device 300, which the handling device 300 was provided by means of another transport vehicle 410, for example during the cleaning of the used tool box 702. This procedure can also be able to be carried out based on a subsequent following packet of the preceding following packet.

Such an above-described exemplary procedure can be able to be carried out repeatedly until all provided and required treatment procedures have been carried out on the educt 40 and the product 20 to be manufactured has been generated from the educt 40. In this case, each procedure is based on a subsequent following packet of the preceding following packet.

The educt container 42 can now be designated as a product container 22, in which the product 20 manufactured from the educt 40 is now located.

The manufactured product 20, which is arranged in a product container 22 arranged in a patient box 30, 50, is removed from the storage rack 404 by means of a further transport vehicle 410 and transported to the transfer airlock space 216 in order to be packaged by means of the packing device 214. This procedure can also be able to be carried out based on a subsequent following packet of the preceding following packet.

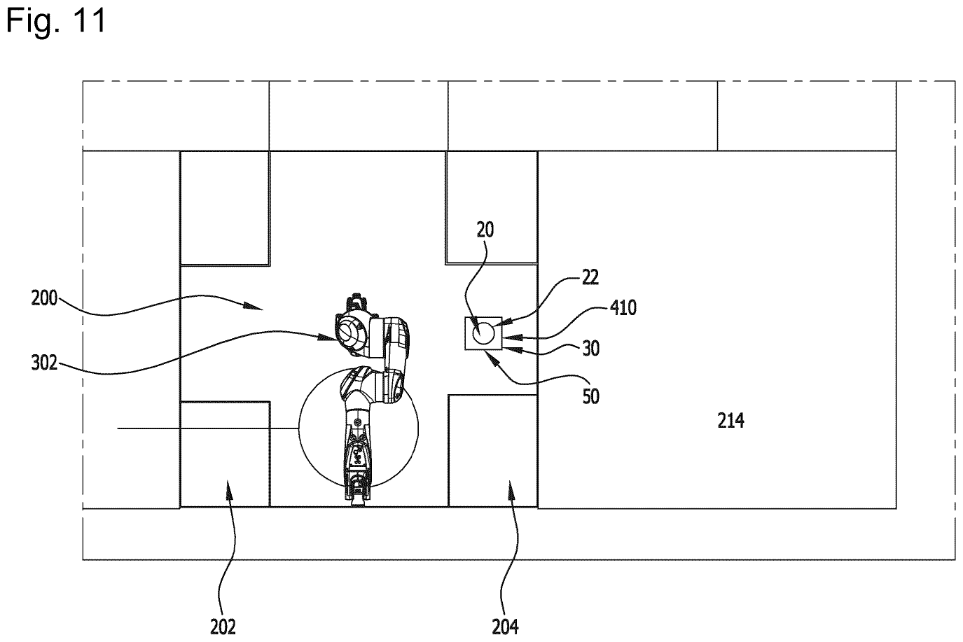

After the packaging by means of the packing device 214, the packaged product container 22 is transferred to the patient box 30, 50 again by means of the multiaxis robot arm 302 (cf. FIG. 11) and fed by means of the transport vehicle 410 to the exit airlock space 208, where the packaged product container 22 having the product 20 is removable by the user.

This procedure can also be able to be carried out based on a subsequent following packet of the preceding following packet.

Alternatively, the entire patient box 30, 50 can also be removable by the user.

With reference to the above description with respect to FIGS. 2 to 11, the exemplary sequence was described without carrying out dummy production program packets as following packets. However, it is to be understood that dummy production program packets can be integratable at any point of the described sequence, in particular by means of the described random module of the production system 10.

Furthermore, it is to be understood that the description with respect to FIGS. 2 to 11 relates to a normal status of the production system 10. In the described sequence, a data connection to the heartbeat server 800 therefore existed continuously or a cessation of the data connection was only so short that the pausing status was not adopted.

The patient boxes 30, 50 and/or the tool boxes 702, 704 each comprise one or more conditioning units (not shown in the figures), which are integrated or detachably arranged thereon or detachably arrangeable thereon, by means of which a temperature and/or a pressure and/or a humidity and/or a gas composition in the interior of the respective patient boxes 30, 50 and/or tool boxes 702, 704 can be influenced, in particular can be controlled and/or regulated.

One or more of the conditioning units are tool units of a or the tool system of the production system 10, which are arrangeable as needed on the respective patient box 30, 50 and/or tool box 702, 704 or removable therefrom, in particular fully automatically by means of the storage transport device 406 and/or by means of the handling device 300, in particular the multiaxis robot arm 302 of the handling device 300.

The patient boxes 30, 50 and/or the tool boxes 702, 704 comprise one or more connection elements, which are connectable to connection elements corresponding thereto on one or more storage spaces 402, in particular to supply the respective patient box 30, 50 and/or tool box 702, 704 with electric energy and/or consumable substances, in particular liquids and/or gases.

The storage spaces 402 are provided for this purpose with connection elements, so that one or more treatment procedures for treating the educts 40 can be carried out at storage spaces 402 of the storage device 400 used as treatment spaces. In particular, these storage spaces 402 are designed as a processing unit.

The storage spaces 402 of the storage device 400 and/or the patient boxes 30, 50 and/or the tool boxes 702, 704 comprise fixing elements, by means of which the patient boxes 30, 50 and/or the tool boxes 702, 704 are positionable and/or fixable at the storage spaces 402.

The patient boxes 30, 50 and/or the tool boxes 702, 704 each comprise one or more tool receptacles for accommodating and securing one or more tools or tool units, in particular in the interior of the respective patient box 30, 50 and/or tool box 702, 704.

The one or the multiple tool receptacles can each comprise one or more connection elements, which are connectable to connection elements corresponding thereto on the respective tool to be accommodated, in particular to supply the respective tool with electric energy and/or consumable substances, in particular liquids and/or gases.

The patient boxes 30, 50 and/or the tool boxes 702, 704 comprise one or more connection elements, which are connectable to connection elements corresponding thereto on a further patient box 30, 50 and/or tool box 702, 704, by which an assembled patient box 30, 50 and/or the tool box 702, 704 can be provided.

The tool boxes 702, 704 are in particular configured to carry out one or more treatment procedures, which can be carried out in particular by means of one or more tool units, and which can comprise the following:
feeding a liquid and/or a gas, in particular a gas mixture,
discharging, in particular pumping out, a liquid and/or a gas, in particular a gas mixture,
discharging, in particular pumping out, a part of a liquid and/or a gas, in particular a gas mixture, for an analysis by means of one or more sensor units,
mixing one or more components, in particular at least one liquid with at least one further component, for example a further liquid,
concentrating and/or separating a liquid in particular by means of a column, for example a separating column, and/or a centrifuge,
analyzing an educt 40 and/or an intermediate product and/or a product 20, in particular by means of one or more sensor units,
storing, in particular placing in and/or removing from storage, one or more objects, in particular by means of the storage transport device 406,
controlling the temperature of, in particular incubating, one or more objects, in particular one or more educts 40.

It is to be understood that the preceding list of the treatment procedures is by way of example and is not exhaustive. Alternatively or additionally, it can be provided that one or more treatment procedures are combinable with one or more treatment procedures in a single treatment procedure and/or in any combination thereof.

With reference to the above description, in particular with respect to FIGS. 2 to 11, it is to be understood that all procedures and/or treatment procedures can be able to be simulated or can be simulated by means of the simulation device, as described herein.

Furthermore, it is to be understood that any measured values originating from sensor units are transmittable or are transmitted in an encrypted and/or abstracted form by means of the respective computing units, as described herein.

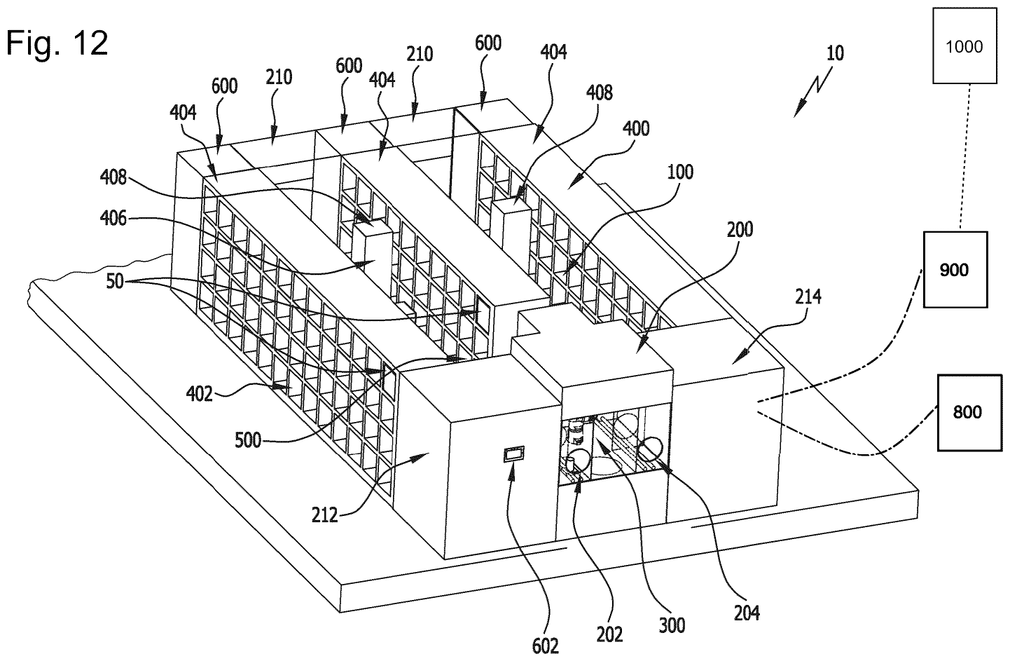

FIG. 12 shows a schematic perspective representation of a production system 10 according to a further exemplary embodiment of the present invention.

The production system 10 according to a further exemplary embodiment of the present invention as shown in FIG. 12 comprises essentially the same structural and functional features as the exemplary embodiment of the production system 10 according to the invention shown in FIG. 1, so that only the structural and functional feature differences are described hereinafter.

It is to be understood that the production system 10 shown in FIG. 12 is operable and/or controllable essentially precisely like the production system according to the invention described at the outset, in particular the production system 10 shown in FIG. 1.

The storage transport device 406 is a rail-guided transport system and comprises one or more, two by way of example here, rail-guided storage transport units 408, by means of which the storage racks 404 of the storage device 400 are accessible in particular for storage and removal procedures and for treatment procedures and/or maintenance procedures.

The storage racks 404 are arranged essentially parallel to one another and each at intervals from one another in the clean room area 100, wherein the storage transport device 406 is arranged in the respective intervals of the storage racks 404, wherein a storage transport unit 408 is assigned to a respective interval between the storage racks 404.

The storage spaces 402 of the middle storage rack 404 are accessible from multiple directions and/or by means of both storage transport units 408 of the storage transport device 406. In particular, the storage spaces 402 of the storage racks 404 are laterally accessible in the horizontal direction by means of the storage transport device 406.

As already described, the storage spaces 402 are configured to accommodate various types of object, wherein the storage spaces 402 can differ from one another with respect to their shape and/or dimensioning. For simplified representation, the storage spaces 402 are solely shown by way of example in FIG. 12 in a uniform manner, in particular in a uniform shape and dimensioning.

The handling device 300 likewise comprises a multiaxis robot arm 302, which is arranged adjacent to the airlock device 200, and an in particular rail-based airlock transport device, by means of which one or more objects are transportable from an airlock space, in particular an entry airlock space 206, to a further airlock space, in particular a transfer airlock space 216, and/or from an airlock space, in particular a transfer airlock space, to a further airlock space, in particular an exit airlock space 208.

By means of the airlock transport device and/or the multiaxis robot arm 302, an educt container 42 is transferable into a patient box 30, 50, which can be provided for this purpose by means of the storage transport unit 408 at the airlock transport device 304.

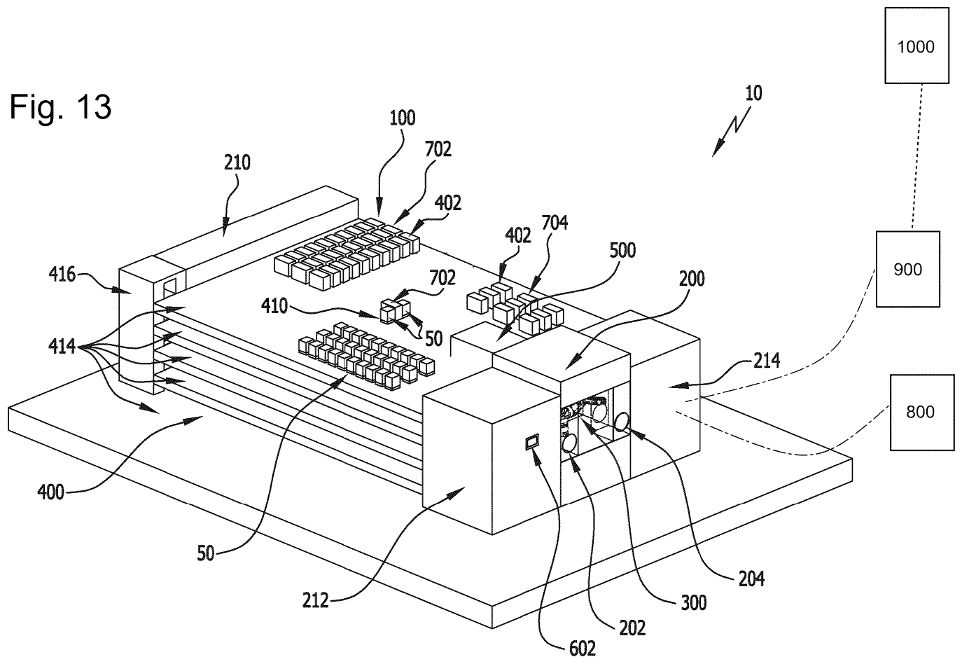

FIG. 13 shows a schematic perspective representation of a production system 10 according to a further exemplary embodiment of the present invention.

The production system 10 according to a further exemplary embodiment of the present invention as shown in FIG. 13 comprises essentially the same structural and functional features as the exemplary embodiment of the production system 10 according to the invention shown in FIG. 1, so that only the structural and functional feature differences are described hereinafter.

It is to be understood that the production system 10 shown in FIG. 13 is operable and/or controllable essentially precisely like the production system according to the invention described at the outset, in particular the production system 10 shown in FIG. 1.

The storage transport device 406 comprises one or more freely moving transport vehicles 410, which were already described in conjunction with the exemplary embodiment of the production system 10 according to the invention shown in FIG. 1.

The storage device 400 comprises multiple, in the present case four here, storage space surfaces 414, which each comprise multiple storage spaces 402, wherein the storage space surfaces 414 are arranged one above another, in particular in a vertical direction. In a certain sense, the storage space surfaces 414 are arranged in a stack, as schematically shown in FIG. 13.

Various types of storage spaces 402 for accommodating various types of objects are arranged on the storage space surfaces 414. As schematically shown in FIG. 13, the various types of storage spaces 402 are distributed in a regular pattern.

For example, multiple, in particular all, patient boxes 50 and multiple, in particular all, tool boxes 702 for and/or having tool units and multiple, in particular all, tool boxes 704 for and/or having consumable materials are arranged spatially adjacent in said pattern.

The storage transport device 406 furthermore comprises one or more elevator devices 416, by means of which the storage racks 414 of the storage device 400 are accessible in particular for storage and removal procedures and for treatment procedures and/or maintenance procedures.

The transport vehicles 410 are movable to the individual storage space surfaces 414 by means of the elevator devices 416.

Additionally or alternatively, it is conceivable that a transfer procedure of a transported object from a transport vehicle 410, which is located on one storage space surface 414, to another transport vehicle 410, which is located on another storage space surface 414, can be carried out by means of the elevator devices 416. In a certain sense, such a transfer procedure from one transport vehicle 410 to another transport vehicle 410 can be carried out via the elevator devices 416.

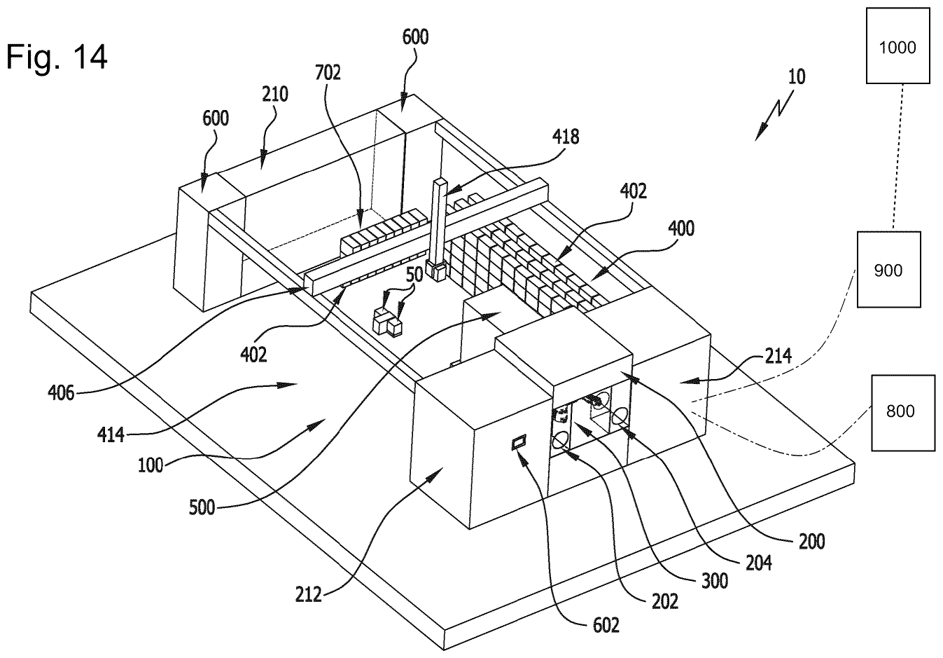

FIG. 14 shows a schematic perspective representation of a production system 10 according to a further exemplary embodiment of the present invention.

The production system 10 according to a further exemplary embodiment of the present invention as shown in FIG. 14 comprises essentially the same structural and functional features as the exemplary embodiment of the production system 10 according to the invention shown in FIG. 1, so that only the structural and functional feature differences are described hereinafter.

It is to be understood that the production system 10 shown in FIG. 14 is operable and/or controllable essentially precisely like the production system according to the invention described at the outset, in particular the production system 10 shown in FIG. 1.

The storage device 400 comprises a storage space surface 414, which comprises multiple storage spaces 402.

Various types of storage spaces 402 for accommodating various types of objects are arranged on the storage space surface 414. As schematically shown in FIG. 14, the various types of storage spaces 402 are distributed in a regular pattern.

For example, multiple, in particular all, patient boxes 50 and multiple, in particular all, tool boxes 702 for and/or having tool units and multiple, in particular all, tool boxes 704 for and/or having consumable materials are arranged spatially adjacent in said pattern.

The storage transport device 406 comprises a storage transport unit 408 designed as a gantry conveyor 418, wherein the storage space surface 414 is accessible by means of the gantry conveyor 418, in particular for storage and removal procedures and also for treatment procedures and/or maintenance procedures.

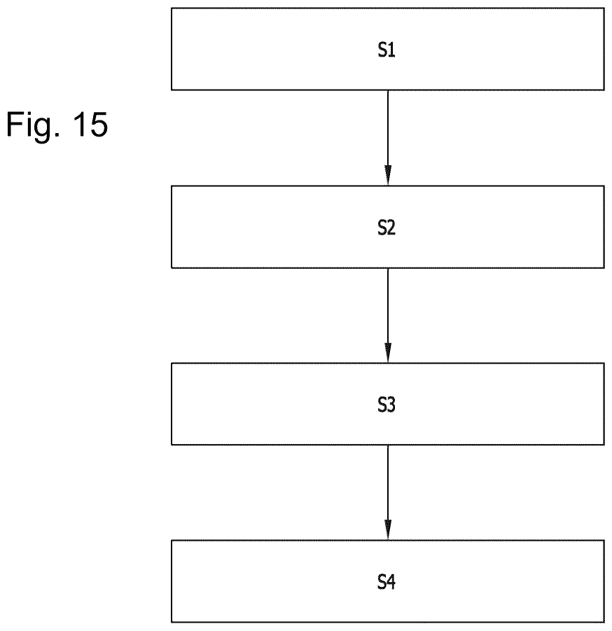

FIG. 15 outlines a method according to an exemplary embodiment of the present invention.

The method is used for the in particular at least partial control of a production system 10 for manufacturing a product 20, in particular a biological-pharmaceutical product. In particular, the method is used for the in particular at least partial control of the production system 10 from FIGS. 1, 12, 13, and/or 14.

The method comprises providing (S1) one or more processing units 30, 50, 702, 704 for accommodating and/or treating an educt 40.

The method furthermore comprises a second, a third, and a fourth step (S2, S3, S4):

The method comprises determining (S2) an in particular encrypted production program packet of a production program, in particular to manufacture a product 20 that can be manufactured by means of the production system 10, and one of the processing units 30, 50, 702, 704 in order to execute this production program packet, based on a production program packet of the production program which precedes the production program packet and is in particular encrypted.

The method comprises activating (S3) the determined production program packet on the determined processing unit 30, 50, 702, 704 in order to decrypt the determined production program packet and then to control the processing unit to carry out one or more treatment steps on the educt based on the decrypted production program packet.

The method comprises determining (S4) a production program packet of the production program which is subsequent to the decrypted production program packet and is in particular encrypted and a subsequent processing unit 30, 50, 702, 704 in order to execute this subsequent production program packet, based on the decrypted production program packet.

The method additionally or alternatively furthermore comprises at least a second, a third, and a fourth additional or alternative step (S2, S3, S4):

The method comprises providing (S2) a data connection from a computer system 800 which is associated or can be associated with the production system to the production system 10 and/or one or more, in particular all processing units 30, 50, 702, 704.

The method comprises determining (S3) whether the data connection exists by means of at least one control unit of the production system 10 and/or at least one computing unit of at least one processing unit 30, 50, 702, 704.

The method comprises controlling (S4) the production system 10 and/or the one or more, in particular all processing units 30, 50, 702, 704 depending on the data connection.

The controlling (S4) comprises a reversible transfer of the production system 10 and/or the one or more, in particular all processing units 30, 50, 702, 704 into a normal status for unrestricted operation, into a reserve status for chronologically and/or functionally restricted operation, and/or into a pausing status for idled operation, in particular based on the existence of the data connection.

The at least one control unit and/or one or more, in particular each of the computing units store an information stamp, in particular a timestamp, upon transfer into the reserve status and stop an unrestricted operation of the production system 10 and/or the one or more, in particular all processing units 30, 50, 702, 704 after a predefined period of time from this information stamp, in particular this timestamp, and transfer the production system 10 and/or the relevant processing units 30, 50, 702, 704 into the pausing status.

The control unit and/or one or more, in particular each of the computing units, upon transfer into the reserve status, only still execute a predefined part of a production program for manufacturing a product 20.

The control unit and/or one or more, in particular each of the computing units, upon transfer into the pausing status, modify and/or supplement a production program for manufacturing a product 20 using one or more production program packets to store an educt 40 and/or an at least partially treated educt 40, i.e. an intermediate product, and/or a product 20, in particular as long as the pausing status exists.

It is to be understood in this case that the respective second, third, and fourth steps (S2, S3, S4) are executable in particular sequentially and/or in parallel.

With respect to the method outlined in FIG. 15, it is to be understood that all structural and/or functional features which are linked with the production system according to the invention and in particular also with its above-described exemplary embodiments of the production system 10, can also be comprised in the method according to the invention either alone or in combination and the properties, designs, and advantages connected thereto can accordingly likewise be comprised and achieved. The above-described production system 10 is moreover configured to execute the method outlined in FIG. 15 and the method outlined in FIG. 15 is furthermore designed to be executable by means of the above-described production system 10.

### LIST OF REFERENCE SIGNS

10 production system
20 product
30 workpiece carrier, tool carrier designed as a patient box
40 educt
42 educt container
50 patient box
100 clean room area
200 airlock device
202 entry airlock
204 exit airlock
206 entry airlock space
208 exit airlock space
210 maintenance airlock
212 unpacking device
214 packing device
216 transfer airlock space
218 waste airlock
300 handling device
302 multiaxis robot arm
400 storage device
402 storage space
404 storage rack
406 storage transport device
408 storage transport units
410 transport vehicle
412 storage rack airlock
414 storage space surface
416 elevator device
418 gantry conveyor
500 cleaning device
600 control supplementary system
602 HMI interface
702 tool box
704 tool box
706 tool path
800 first computer system, heartbeat server
900 second computer system, release server
1000 (database access) authorized party, separate computer system
S1 first method step
S2 second method step
S3 third method step
S4 fourth method step

1. A production system for manufacturing a product, or a biological-pharmaceutical product, the production system comprising:
multiple processing units for accommodating and/or treating an educt, wherein each processing unit comprises at least one computing unit; and
at least one control unit, which is designed and programmed to determine an integrity of each processing unit or an integrity of each processing unit before an educt can be accommodated and/or treated by means of a processing unit according to one of multiple production programs for manufacturing one of multiple products that can be manufactured by means of the production system.

2. The production system as claimed in claim 1,
wherein the computing unit of a respective processing unit is designed and programmed to provide a first unique item of information related to said processing unit, and a second unique item of information related to said processing unit is detectable by means of the at least one control unit, and
wherein the integrity of said processing unit is determinable based on a comparison of the first and second unique items of information by means of the at least one control unit and/or is settable and/or confirmed upon correspondence of the first and second unique items of information.

3. The production system as claimed in claim 1,
wherein a processing unit comprises a security module or a trusted platform module for providing a unique item of information or an item of fingerprint information about the processing unit, and
wherein the computing unit of the processing unit is designed and programmed to provide the unique item of information of the at least one control unit to determine the integrity.

4. The production system as claimed in claim 1, wherein
the production system comprises one or more sensor units, by means of which the processing units are visually detectable, and on the basis of this detection a unique item of information with respect to the respective processing units is provided by means of the at least one control unit to determine the integrity.

5. The production system as claimed in claim 4, wherein
the one or more sensor units comprise one or more camera units, by means of which characteristic landmarks of a face or a surface and/or a geometry of the processing units are visually detectable.

6. The production system as claimed in claim 4, wherein
the production system comprises one or more actuator units, by means of which one or more processing units are mechanically excited or mechanically excited to generate a mechanical oscillation, which is detectable by means of the one or more sensor units and on the basis of which a unique item of information with respect to the respective processing units can be provided by means of the at least one control unit to determine the integrity.

7. The production system as claimed in claim 6, wherein
at least one of the one or more actuator units is configured to generate the mechanical oscillation and said actuator unit is arranged on or in one of the one or more processing units and/or can be engaged with one of the one or more processing units to generate the mechanical oscillation on the relevant processing unit.

8. The production system as claimed in claim 6, wherein
the oscillation comprises a frequency range of greater than or equal to 10 Hz and less than or equal to 100 Hz, and/or the oscillation comprises one or more different frequency ranges, which are respectively greater than or equal to 10 Hz and less than or equal to 100 Hz, and/or the oscillation comprises said one or more different frequency ranges which follow one another in a specific order.

9. The production system as claimed in claim 6, wherein
at least one of the one or more actuator units is configured to generate an acoustic oscillation or a soundwave and said actuator unit is arranged on or in one of the one or more processing units and/or can be engaged with one of the one or more processing units to generate the mechanical oscillation or the soundwave with respect to the relevant processing unit.

10. The production system as claimed in claim 9, wherein
the oscillation comprises a frequency range of greater than or equal to 30 Hz and less than or equal to 10000 Hz, and/or the oscillation comprises one or more different frequency ranges, which are respectively greater than or equal to 30 Hz and less than or equal to 10000 Hz, and/or the oscillation comprises said one or more different frequency ranges which follow one another in a specific order.

11. The production system as claimed in claim 1,
wherein one or more or all processing units comprise at least one receptacle chamber, or at least one receptacle chamber enclosed by a lateral surface, for accommodating one or more objects, or an educt and/or one or more objects for treating the educt, and
wherein the integrity comprises an internal integrity which relates to the receptacle chamber and/or comprises an external integrity which relates to an exterior with respect to the receptacle chamber.

12. The production system as claimed in claim 1, wherein
a processing unit is designed and programmed to execute instructions according to at least one production program, and/or to decrypt and then execute them, if its integrity is set and/or confirmed.

13. The production system as claimed in claim 12,
wherein a production program comprises multiple encrypted production program packets, wherein each production program packet contains at least one item of information which refers to another production program packet and/or comprises instructions for accommodating and/or treating an educt, and
wherein:
a) one or more or all of the production program packets are provided on one processing unit in each case and can only be executed, or decrypted and executed, if the integrity of the relevant processing unit is set and/or confirmed; and/or
b) one or more or all of the production program packets are transmittable to one processing unit in each case, and/or by means of the at least one control unit and/or by means of a computer system which is associated or can be associated with the production system, if the integrity of the relevant processing unit is set and/or confirmed.

14. The production system as claimed in claim 1, wherein
a processing unit is designed and programmed to block an execution and/or a decryption of instructions according to at least one production program if their integrity cannot be set and/or cannot be confirmed.

15. The production system as claimed in claim 1, wherein
a processing unit is designed and programmed to provide a message or an alarm message or to the at least one control unit and/or a computer system which is associated or can be associated with the production system, if their integrity cannot be set and/or cannot be confirmed.
